(19) United States
(12) Patent Application Publication
(10) Pub. No.: US 2012/0196889 A1
(43) Pub. Date: Aug. 2, 2012

(54) CATECHOLAMINE DERIVATIVES AND PRODRUGS THEREOF

(75) Inventors: Benny Bang-Andersen, Copenhagen S. (DK); Mogens Larsen, Smorum (DK); Klaus Gundertofte, Frederiksberg (DK); Morten Jorgensen, Bagsvaerd (DK); Jennifer Larsen, Roskilde (DK); Niels Mork, Virum (DK); HåKan Wikström, Hamburgsund (SE)

(73) Assignee: H. Lundbeck A/S, Valby-Copenhagen (DK)

(21) Appl. No.: 13/202,579

(22) PCT Filed: Feb. 24, 2010
(86) PCT No.: PCT/DK2010/050046

§ 371 Date: Dec. 18, 2011

(30) Foreign Application Priority Data
Feb. 25, 2009 (DK) PA200900252

(51) Int. Cl. A61K 31/4741; A61K 31/407; C07D 209/80; C07D 491/056; C07D 221/06; A61K 31/473; A61K 31/404

(52) U.S. Cl. 514/287; 514/290; 514/410; 514/411; 546/65; 546/101; 548/421; 548/427

(57) ABSTRACT

The present invention relates to novel catecholamine derivatives of Formula I, to processes for their preparation, pharmaceutical compositions containing them and to their use in therapy.

### FIELD OF THE INVENTION

Aspects of the present invention relate to novel catecholamines and catecholamine derivatives, to processes for their preparation, pharmaceutical compositions containing them and their use in therapy.

### BACKGROUND ART

Neurodegenerative diseases such as Alzheimer's and Huntington's disease are becoming more prevalent with the aging population. One particular neurodegenerative disease, which typically has its onset between the ages of 50 and 80 years of age, is Parkinson's disease (PD). PD is a disorder of the brain, which is characterized by tremor and difficulty with walking, movement, and coordination.

Dopamine (DA) is a chemical neurotransmitter, which is utilized by brain cells to transmit impulses to control or modulate peripheral muscle movement. PD is believed to be caused by a progressive deterioration of DA-containing neurons in the substantia nigra zona compacta of the brain. The degeneration of the DA-containing neurons results in reduced amounts of DA in the brain. This process is thought to disturb the nerve cell function such that impulses are not transmitted properly, resulting in a loss of muscle control and function.

Currently, there is no cure for PD. Treatments are typically aimed at controlling the PD symptoms, primarily by replacing the DA with either (levo)-3,4-dihydroxy phenylalanine (L-DOPA) which is metabolized to DA, or by administering chemical agents that stimulate the DA receptors. These receptors fall into two broad classes, D1-type and D2-type receptors. The former is divided into D1 and D5 receptors, while the D2 receptor family consists of D2, D3, and D4 receptors.

Certain hydroxylated (phenols or catechols) phenylethylamines (as such or forming part of a semirigid/rigid ring system) are known to possess dopaminergic activity at least in animal models. However, their clinical use is limited because they have low or no oral bioavailability, most likely due to their high first-pass metabolism. However, Apomorphine, which belongs to this class of compounds, is used clinically in PD therapy albeit with a non-oral delivery (typically intermittent subcutaneous administration or daytime continuous infusion). Several clinical studies are ongoing with alternative delivery strategies for Apomorphine therapy in PD such as intranasal and sublingual formulations. However these efforts are yet to result in an option for the clinical treatment of PD.

Direct DA receptor agonists are able to activate the DA autoreceptors as well as the postsynaptic DA receptors. The effects of autoreceptor stimulation appear to predominate when Apomorphine is administered at low doses, whereas at higher doses the attenuation of DA transmission is outweighed by the enhancement of postsynaptic receptor stimulation. The antipsychotic effects in man of low doses of Apomorphine are likely due to the autoreceptor stimulation (for a discussion of clinical data, see: Tamminga; J. Neurol. Trans., 2002, 109(3), 411).

L-DOPA is an efficacious PD drug (a prodrug of dopamine) with a poor PK profile leading to dyskinesia and other response fluctuations. Selective D2-agonists (e.g. Pramipexole) give less dyskinesia, but lack efficacy in late PD and eventually need complementation or replacement with L-DOPA. L-DOPA and Apomorphine are currently the most efficacious PD drugs and they stimulate both D1 and D2 receptors.

As mentioned previously, the poor oral bioavailability of catecholamines has prevented their clinical use as oral drugs. The phenolic amines have similar poor oral bioavailability limiting their clinical use as orally active drugs. However, Rotigotine, which belongs to this class of compounds, was recently introduced as a new PD drug based on a transdermal delivery. For Apomorphine, animal studies have shown that transdermal delivery or via implants may provide possible forms of administration. However, when the delivery of Apomorphine from implants was studied in monkeys (Bibbiani, et al. Chase Experimental Neurology 2005, 192, 73] it was found that in most cases the animals had to be treated with the immunosuppressant Dexamethasone to prevent local irritation and other complications following the implantation surgery. Transdermal delivery of Apomorphine has also been associated with local skin irritation and coloration.

Apart from PD, other diseases in which an increase in dopaminergic turnover may be beneficial are geriatrics, for preventing bradykinesia and depression and in the improvement of mental functions including various aspects of cognition as discussed above. It can have a positive effect in depressed patients, and it can be used in obesity as an anorectic agent. It can improve minimal brain dysfunction (MBD), narcolepsy, and potentially the negative, the positive as well as the cognitive symptoms of schizophrenia. Restless leg syndrome (RLS) and periodic limb movement disorder (PLMD) are alternative indications, which are clinically treated with DA-agonists. In addition, impotence and erectile dysfunction are also likely to be improved by treatment with DA-agonists. Thus, improvement of sexual functions in both women and men is another possible indication for treatment with DA-agonists since erectile dysfunction (impotence in men) and sexual stimulation in e.g. menopausal women (stimulation of vaginal lubrication and erection of clitoris) potentially can be achieved via DA-receptor stimulation. In this context, it is noteworthy that Apomorphine when given sublingually is used clinically to improve erectile dysfunction. Clinical studies of L-DOPA and the D2 agonist Pramipexole therapy in Huntington's disease have shown promising results; thus treatment of Huntington's disease is another potential application of the compounds of the invention. DA is involved in regulation of the cardiovascular and renal systems, and accordingly, renal failure and hypertension can be considered alternative indications for the compounds of the invention.

An alternative to the non-oral formulations of the catecholamines involves the use of a prodrug. A problem associated with the development of such compounds for clinical use is the difficulties associated with predicting conversion to the catecholamine itself in humans. Various ester prodrugs of catecholamines have been reported in the literature such as enterically coated NPA esters for duodenal delivery (see eg. Wikström, Dijkstra, Cremers, Ivo; WO 02100377), and the D1-like agonist Adrogolide (ABT-431; DAS-431, a diacetyl prodrug of A-86929). Adrogolide undergoes a high hepatic first-pass metabolism in man after oral dosing and, as a result, has a low oral bioavailability (app. 4%). In PD patients, intravenous (IV) Adrogolide has antiparkinson efficacy comparable to that of L-DOPA [Giardina, Williams; CNS Drug Reviews, 2001, 7, 305. An alternative approach involves the ‘masking’ of the two hydroxyl groups in the catechol as the corresponding methylene-dioxy (MDO) acetal, as the acetal derived from other aldehydes than formaldehyde, or as the ketal derived from various ketones. This prodrug principle has been reported for the Aporphines more than 20 years ago (Baldessarini, et al. Neuroropharmacology, 1982, 21(10), 953). Of these potential prodrugs to Apomorphine and related compounds, only that derived from N-n-propyl Apomorphine (NPA) and formaldehyde showed significant efficacy in animal models of PD. Over the following ˜25 years, these findings have not lead to a PD drug based on the MDO-masked Apomorphines or related compounds.

Despite the long-standing interest in the field, there is evidently still an unmet need as regards developing efficient, well-tolerated and orally active drugs for the treatment of PD. A mixed D1-like/D2-like agonist giving continuous dopaminergic stimulation may fulfil such unmet needs.

### SUMMARY OF THE INVENTION

Aspects of the present invention are concerned with the compounds of Formula I:

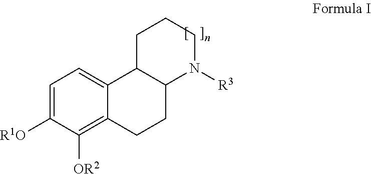

wherein is n is 0 or 1;
wherein R¹ and R² are independently selected from the group consisting of hydrogen, C₁₋₆ alkanoyl, phenylacetyl or benzoyl, or wherein R¹ and R² fuse to form a methylene (CH₂) group, a carbonyl (C═O) group or an oxalyl (O═C—C═O) group; and
wherein R³ is selected from the group consisting of hydrogen, methyl, ethyl, n-propyl, cyclopropyl, cyclobutyl, cycloalkylalkyl, allyl, propargyl, hydroxyethyl, benzyl or phenylethyl, where the benzyl and phenylethyl are optionally substituted with C₁-C₆ alkyl or halogen; or a pharmaceutically acceptable acid addition salt thereof;
provided that the compound is not the racemic mixture of one of the following compounds:
1,2,3,4,4a,5,6,10b-octahydro-benzo[f]quinoline-7,8-diol,
4-methyl-1,2,3,4,4a,5,6,10b-octahydro-benzo[f]quinoline-7,8-diol,
4-ethyl-1,2,3,4,4a,5,6,10b-octahydro-benzo[f]quinoline-7,8-diol,
4-n-propyl-1,2,3,4,4a,5,6,10b-octahydro-benzo[f]quinoline-7,8-diol,
4-benzyl-1,2,3,4,4a,5,6,10b-octahydro-benzo[f]quinoline-7,8-diol, and
4-phenylethyl-1,2,3,4,4a,5,6,10b-octahydro-benzo[f]quinoline-7,8-diol.

In separate aspects, the compound is selected from one of the exemplified compounds disclosed in the Experimental Section.

Furthermore, the present invention provides a pharmaceutical composition comprising a therapeutically effective amount of a compound of Formula I and a pharmaceutically acceptable carrier. The present invention also provides a process for making a pharmaceutical composition comprising admixing a therapeutically effective amount of a compound of Formula I and a pharmaceutically acceptable carrier.

The subject invention relates to the use of the compound of Formula I or a pharmaceutically acceptable acid addition salt thereof for the preparation of a medicament for the treatment of Parkinson's disease or Huntington's disease in a mammal.

A separate aspect is directed to the use of the compound of Formula I or a pharmaceutically acceptable acid addition salt thereof for the preparation of a medicament for the treatment of psychoses, impotence, renal failure, heart failure, or hypertension in a mammal.

The present invention further provides for the use of the compound or Formula I of a pharmaceutically acceptable acid addition salt thereof, for the manufacture of a medicament for the treatment of cognitive impairment in a mammal.

Another aspect is directed to the use of the compound of Formula I or a pharmaceutically acceptable acid addition salt thereof, for the manufacture of a medicament for the treatment of restless legs syndrome (RLS) or periodic limb movement disorder (PLMD) in a mammal.

One aspect is directed to the use of the compound of Formula I or a pharmaceutically acceptable acid addition salt thereof, for the manufacture of a medicament for the treatment of movement disorders, poverty of movement, dyskinetic disorders, gait disorders or intention tremor in a mammal.

Yet another aspect is directed to the use of the compound or Formula I of a pharmaceutically acceptable acid addition salt thereof, for the manufacture of a medicament for the treatment of dyskinesias in a mammal. One aspect is directed to the use of the compound of Formula I or a pharmaceutically acceptable acid addition salt thereof, for the manufacture of a medicament for the treatment of depression, bipolar disorder and anxiety in a mammal.

Yet another aspect is directed to the use of the compound of Formula I or a pharmaceutically acceptable acid addition salt thereof, for the manufacture of a medicament for the treatment of cognitive impairment associated with a disorder or disease selected from schizophrenia, Parkinson's Disease, dementia such as AIDS dementia, anxiety disorder, age associated memory impairment, depression, including major depression, in particular in elderly, Alzheimer's Disease, attention deficit hyperactivity disorder (ADHD) or post-traumatic stress disorder (PTSD) in a mammal.

Additionally, the present invention is also directed to methods of treating the disorders mentioned above comprising administering a therapeutically effective amount of the compound of Formula I or a pharmaceutically acceptable acid addition salt thereof.

### DETAILED DESCRIPTION

The compounds of the present invention contain two chiral centers (denoted with * in the formula below).

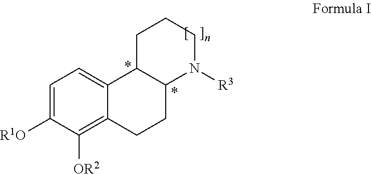

The compounds of the invention (Formula I) can exist in two different diastereomeric forms, the cis- and trans-isomers. The diastereomeric forms further comprise two enantiomeric forms each, which means that the compounds of Formula I overall exist as the individual (R,R), (R,S), (S,S) and (S,R) enantiomers. A racemic mixture consists of the cis- and trans-isomers.

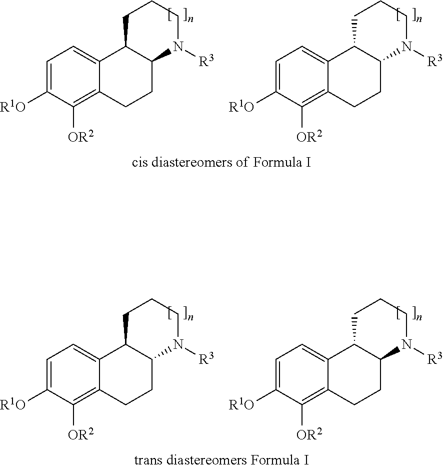

The compounds of Formula I are expected to behave like orally active Apomorphine-analogues, which render them potentially useful in relation to treatment of Parkinson's disease and other diseases/disorders, which responds favorably to an increased dopaminergic turnover.

In one embodiment, R³ is selected from the group consisting of hydrogen, methyl, ethyl, n-propyl, allyl, and propargyl.

In one embodiment, R³ is selected from the group consisting of cyclo-propyl, cyclo-butyl, and hydroxyethyl.

In one embodiment, n is 0. In a separate embodiment, n is 1.

In one embodiment, the compound is characterized as the substantially pure trans-diastereoisomer.

In another embodiment, R¹ and R² are fused and form a methylene (CH₂) group.

In a separate embodiment, n is 0 and the compound is further characterized as the substantially pure (3aS,9bR)-enantiomer.

In one embodiment, n is 0 and the compound is further characterized as the substantially pure (3aS,9bS)-enantiomer.

In yet another embodiment, n is 1 and the compound is further characterized as the substantially pure (4aS,10bR)-enantiomer.

In one embodiment, n is 1 and the compound is further characterized as the substantially pure (4aS,10bS)-enantiomer.

Another embodiment relates to the free base of a compound of Formula I, or a salt hereof, or a pharmaceutical composition hereof and the uses as described herein, wherein the compound of Formula I has a trans-diastereomeric excess of at least 10% (10% trans-diastereomeric excess means that the ratio of the trans- to the cis-diastereoisomer is 55:45 in the mixture in question), at least 25%, at least 50%, at least 70%, at least 80%, at least 90%, at least 95%, at least 97%, preferably at least 98%.

In one embodiment, the compound is selected from the group consisting of (6aR,10aR)-6,6a,7,8,9,10,10a,11-octahydro-1,3-dioxa-7-aza-cyclopenta[a]anthracene; (6aR,10aR)-7-methyl-6,6a,7,8,9,10,10a,11-octahydro-1,3-dioxa-7-azacyclopenta[a]anthracene; (6aR,10aR)-7-ethyl-6,6a,7,8,9,10,10a,11-octahydro-1,3-dioxa-7-aza-cyclopenta[a]anthracene; and (6aR,10aR)-7-n-propyl-6,6a,7,8,9,10,10a,11-octahydro-1,3-dioxa-7-aza-cyclopenta[a]anthracene, or a pharmaceutically acceptable acid addition salt thereof.

In one embodiment, R¹ and R² are fused and form a methylene (CH₂) group, and R₃ is selected from the group consisting of hydrogen, methyl, ethyl and n-propyl, such as methyl and n-propyl.

In one embodiment, R¹ and R² are fused and form a methylene (CH₂) group, and R₃ is selected from the group consisting of hydrogen, methyl, ethyl and n-propyl.

One embodiment is directed to the use of a compound of Formula I as a medicament.

In one embodiment, the compound is (5aR,8aR)-5,5a,6,7,8,8a-hexahydro-4H-1,3-dioxa-6-aza-dicyclopenta[a,f]naphthalene or (5aS,8aS)-5,5a,6,7,8,8a-hexahydro-4H-1,3-dioxa-6-aza-dicyclopenta[a,f]naphthalene.

In one embodiment, the compound is (5aR,8aR)-6-ethyl-5,5a,6,7,8,8a-hexahydro-4H-1,3-dioxa-6-aza-dicyclopenta[a,f]naphthalene or (5aS,8aS)-6-ethyl-5,5a,6,7,8,8a-hexahydro-4H-1,3-dioxa-6-aza-dicyclopenta[a,f]naphthalene.

In one embodiment, the compound is (5aR,8aR)-6-n-propyl-5,5a,6,7,8,8a-hexahydro-4H-1,3-dioxa-6-aza-dicyclopenta[a,f]naphthalene or (5aS,8aS)-6-n-propyl-5,5a,6,7,8,8a-hexahydro-4H-1,3-dioxa-6-aza-dicyclopenta[a,f]naphthalene.

In one embodiment, the compound is (4aS,10bR)-1,2,3,4,4a,5,6,10b-Octahydro-benzo[f]quinoline-7,8-diol or (4aR,10bS)-1,2,3,4,4a,5,6,10b-octahydro-benzo[f]quinoline-7,8-diol.

In one embodiment, the compound is (4aS,10bR)-4-methyl-1,2,3,4,4a,5,6,10b-octahydro-benzo[f]quinoline-7,8-diol or (4aR,10bS)-4-methyl-1,2,3,4,4a,5,6,10b-octahydro-benzo[f]quinoline-7,8-diol.

In one embodiment, the compound is (4aS,10bR)-4-ethyl-1,2,3,4,4a,5,6,10b-octahydro-benzo[f]quinoline-7,8-diol or (4aR,10bS)-4-ethyl-1,2,3,4,4a,5,6,10b-octahydro-benzo[f]quinoline-7,8-diol.

In one embodiment, the compound is (4aS,10bR)-4-propyl-1,2,3,4,4a,5,6,10b-octahydro-benzo[f]quinoline-7,8-diol or (4aR,10bS)-4-propyl-1,2,3,4,4a,5,6,10b-octahydro-benzo[f]quinoline-7,8-diol.

In one embodiment, the compound is (4aS,10bR)-4-benzyl-1,2,3,4,4a,5,6,10b-octahydro-benzo[f]quinoline-7,8-diol or (4aR,10bS)-4-benzyl-1,2,3,4,4a,5,6,10b-octahydro-benzo[f]quinoline-7,8-diol.

In one embodiment, the compound is (4aR,10bR)-4-methyl-1,2,3,4,4a,5,6,10b-octahydro-benzo[f]quinoline-7,8-diol or (4aS,10bS)-4-methyl-1,2,3,4,4a,5,6,10b-octahydro-benzo[f]quinoline-7,8-diol.

In one embodiment, the compound is (4aR,10bR)-4-ethyl-1,2,3,4,4a,5,6,10b-octahydro-benzo[f]quinoline-7,8-diol or (4aS,10bS)-4-ethyl-1,2,3,4,4a,5,6,10b-octahydro-benzo[f]quinoline-7,8-diol.

In one embodiment, the compound is (4aR,10bR)-4-cyclo-butyl-1,2,3,4,4a,5,6,10b-octahydro-benzo[f]quinoline-7,8-diol or (4aS,10bS)-4-cyclo-butyl-1,2,3,4,4a,5,6,10b-octahydro-benzo[f]quinoline-7,8-diol.

In one embodiment, the compound is (4aR,10bR)-4-benzyl-1,2,3,4,4a,5,6,10b-octahydro-benzo[f]quinoline-7,8-diol or (4aS,10bS)-4-benzyl-1,2,3,4,4a,5,6,10b-octahydro-benzo[f]quinoline-7,8-diol.

In one embodiment, the compound is (4aR,10bR)-4-(3-chloro-benzyl)-1,2,3,4,4a,5,6,10b-octahydro-benzo[f]quinoline-7,8-diol or (4aS,10bS)-4-(3-chloro-benzyl)-1,2,3,4,4a,5,6,10b-octahydro-benzo[f]quinoline-7,8-diol.

In one embodiment, the compound is (5S,10S)-4-propyl-1,2,3,4,5,6,7,10-octahydro-15,17-dioxa-4-aza-cyclopenta[a]phenanthrene or (5R,10R)-4-propyl-1,2,3,4,5,6,7,10-octahydro-15,17-dioxa-4-aza-cyclopenta[a]phenanthrene.

In one embodiment, the compound is 2,2-Dimethyl-propionic acid (4aS,10bS)-8-(2,2-dimethyl-propionyloxy)-4-propyl-1,2,3,4,4a,5,6,10b-octahydro-benzo[f]quinolin-7-yl ester or 2,2-dimethyl-propionic acid (4aR,10bR)-8-(2,2-dimethyl-propionyloxy)-4-propyl-1,2,3,4,4a,5,6,10b-octahydro-benzo[f]quinolin-7-yl ester.

As mentioned previously, the compound Apomorphine is currently used clinically in PD therapy. Apomorphine is a mixed D1-like/D2-like agonist:

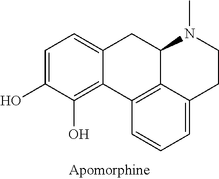

When the compounds of the invention are tested in vitro and in vivo for their effect on D1 and D2 receptors, their pharmacological profiles are very different from that of Apomorphine (see Experimental Section for details)

The presently available information supports the hypothesis that a D1-like agonist (be it selective for either subtype or a mixed D1/D5 agonist) could have important applications in the treatment of cognitive impairment in e.g. psychosis, PD, and Alzheimer's disease (AD), and Huntington's disease. This might well be the case also for dual action D1/D2 agonists, such as the compounds of Formula I.

In another aspect the present invention comprises compounds of Formula I wherein the catechol moiety is masked as a methylenedioxy (MDO) prodrug derivative, which may be cleaved in vivo (most likely by in vivo metabolism) to generate the active catecholamines (exemplified below for n=1):

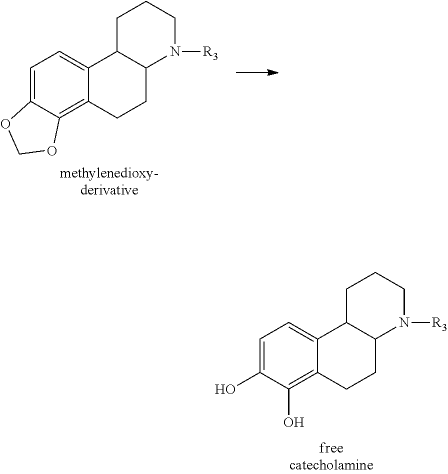

The invention thus also relates to compounds of Formula I wherein R₁ and R₂ are fused and form a methylene (CH₂) group.

In another aspect the present invention also comprise such compounds of Formula I wherein the catechol moiety is masked as a di-ester derivative which may also be cleaved in vivo to generate the active catecholamines (exemplified below for n=1, and R₁ and R₂=acetyl):

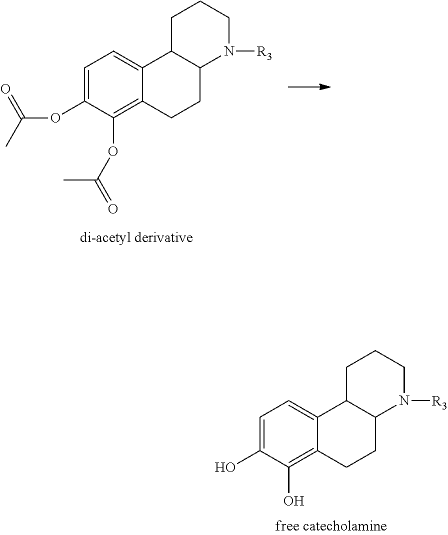

The present invention further comprises unsymmetrical di-ester derivatives of the compounds of Formula I, wherein R₁ and R₂ are two different substituents. The present invention also comprises compounds wherein R₁ and R₂ are fused and form a carbonyl (C═O) group, such that a cyclic di-ester (a carbonate) is produced.

Additionally, one aspect of the invention provides the use of a compound of Formula I or a pharmaceutically acceptable acid addition salt thereof for the preparation of a medicament for the treatment of neurodegenerative disorders such as Parkinson's disease and Huntington's disease.

In a further aspect the invention provides the use of a compound of Formula I or a pharmaceutically acceptable acid addition salt thereof for the preparation of a medicament for the treatment of psychoses, impotence, renal failure, heart failure or hypertension.

In another aspect the invention provides the use of a compound of Formula I, or a pharmaceutically acceptable acid addition salt thereof, for the manufacture of a medicament for the treatment of cognitive impairment in a mammal.

In a still further aspect the invention provides the use of a compound of Formula I, or a pharmaceutically acceptable acid addition salt thereof, for the manufacture of a medicament for the treatment of restless legs syndrome (RLS) or periodic limb movement disorder (PLMD).

In a different aspect the invention provides the use of a compound of Formula I, or a pharmaceutically acceptable acid addition salt thereof, for the manufacture of a medicament for the treatment of movement disorders, poverty of movement, dyskinetic disorders, gait disorders or intention tremor in a mammal.

In separate aspects, the invention provides the use of a compound of Formula I, or a pharmaceutically acceptable acid addition salt thereof, for the manufacture of medicaments, which are intended for oral administration, or for non-oral administration.

A specific embodiment of the present invention relates to the use of a compound of Formula I or a pharmaceutically acceptable addition salt thereof for improving cognition in a mammal in a condition of cognitive impairment wherein the condition is associated with schizophrenia. In another embodiment of the invention the condition is associated with Parkinson's disease. In another embodiment of the invention the condition is associated with dementia, such as AIDS dementia. In another embodiment of the invention the condition is associated with an anxiety disorder. In another embodiment of the invention the condition is associated with age associated memory impairment. In another embodiment of the invention the condition is associated with depression, including major depression, in particular in elderly. In another embodiment of the invention the condition is associated with the use of benzodiazepines. In another embodiment of the invention the condition is associated with the use of tricyclic antidepressants. In another embodiment of the invention the condition is associated with Alzheimer's disease. In another embodiment of the invention the condition is associated with attention deficit hyperactivity disorder (ADHD). In another embodiment of the invention the condition is associated with post-traumatic stress disorder (PTSD).

In a further embodiment the present invention relates to the use of a compound of Formula I or a pharmaceutically acceptable addition salt thereof for the treatment of dyskinesias in a mammal.

In another embodiment the present invention relates to the use of a compound of Formula I or a pharmaceutically acceptable addition salt thereof for the treatment of a mammal suffering from depression, such as major depression, bipolar disorder or anxiety.

The invention also provides a method of treating a mammal suffering from a neurodegenerative disorder such as Parkinson's disease and Huntington's disease comprising administering to the mammal a therapeutically effective amount of a compound of Formula I, or a pharmaceutically acceptable acid addition salt thereof.

In another aspect the invention also provides a method of treating a mammal suffering from psychoses, impotence, renal failure, heart failure or hypertension, comprising administering to the mammal a therapeutically effective amount of a compound of a compound of Formula I, or a pharmaceutically acceptable acid addition salt thereof.

In a further aspect the invention provides a method of treating a mammal suffering from a cognitive impairment, comprising administering to the mammal an effective amount of a compound of Formula I, or a pharmaceutically acceptable acid addition salt thereof.

The invention also relates to a method of treating a mammal suffering from restless legs syndrome (RLS) or periodic limb movement disorder (PLMD), comprising administering to the mammal a therapeutically effective amount of a compound of Formula I, or a pharmaceutically acceptable addition salt thereof.

The invention also relates a method of treating a mammal suffering from movement disorders, poverty of movement, dyskinetic disorders, gait disorders or intention tremor comprising administering to the mammal a therapeutically effective amount of a compound of Formula I, or a pharmaceutically acceptable acid addition salt thereof.

In a specific embodiment of the invention the mammal is a human subject.

The therapeutically effective amount of a compound of Formula I, calculated as the daily dose of the compound of Formula (I) above as the free base, is suitably between 0.01 and 125 mg/day, more suitable between 0.05 and 100 mg/day, e.g. preferably between 0.1 and 50 mg/day.

In a specific embodiment the daily dose of the compound of Formula I is between 1 and 10 mg/day.

In another embodiment the daily dose of the compound of Formula I is less than about 1 mg/day.

In a separate embodiment the daily dose of the compound of Formula I is about 0.1 mg/day.

In a further embodiment the invention provides an oral formulation comprising from 0.001 mg to 125 mg of a compound of Formula I.

In a further embodiment the invention provides an oral formulation comprising from 0.001 mg to 0.1 mg of a compound of Formula I.

In a further embodiment the invention provides an oral formulation comprising from 0.01 mg to 1 mg of a compound of Formula I.

In a further embodiment the invention provides an oral formulation comprising from 0.1 mg to 10 mg of a compound of Formula I.

Furthermore, the present invention provides a pharmaceutical composition comprising a therapeutically effective amount of a compound of Formula I and a pharmaceutically acceptable carrier. The present invention also provides a process for making a pharmaceutical composition comprising admixing a therapeutically effective amount of a compound of Formula I and a pharmaceutically acceptable carrier.

The compound of Formula I, either as the free base, or as a pharmaceutically acceptable acid addition salt, or as a pharmaceutical composition, may be administered in any suitable way e.g. orally, buccally, sublingually, non-orally or parenterally, and the compound may be presented in any suitable form for such administration, e.g. orally in the form of tablets, capsules, powders, syrups, solutions or dispersions, non-orally in the form of eg. transdermal patches or parenterally in the form of dispersions or solutions for injection. In one embodiment, the compound of Formula I is administered in the form of a solid pharmaceutical entity, suitably as a tablet or a capsule.

The compounds of Formula I form pharmaceutically acceptable acid addition salts with a wide variety of organic and inorganic acids. Such salts are also part of this invention.

A pharmaceutically acceptable acid addition salt of the compound of Formula I is formed from a pharmaceutically acceptable acid as is well known in the art. Such salts include the pharmaceutically acceptable salts listed in Journal of Pharmaceutical Science, 66, 2-19 (1977) and are known to the skilled person. Typical inorganic acids used to form such salts include hydrochloric, hydrobromic, hydriodic, nitric, sulphuric, phosphoric, hypophosphoric, metaphosphoric, pyrophosphoric, and the like. Salts derived from organic acids, such as aliphatic mono and dicarboxylic acids, phenyl substituted alkanoic acids, hydroxyalkanoic and hydroxyalkandioic acids, aromatic acids, aliphatic and aromatic sulfonic acids, may also be used. Such pharmaceutically acceptable salts thus include the chloride, bromide, iodide, nitrate, acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, isobutyrate, phenylbutyrate, α-hydroxybutyrate, butyne-1,4-dicarboxylate, hexyne-1,4-dicarboxylate, caprate, caprylate, cinnamate, citrate, formate, fumarate, glycollate, heptanoate, hippurate, lactate, malate, maleate, hydroxymaleate, malonate, mandelate, mesylate, nicotinate, isonicotinate, oxalate, phthalate, teraphthalate, propiolate, propionate, phenylpropionate, salicylate, sebacate, succinate, suberate, benzenesulfonate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, p-toluenesulfonate, xylenesulfonate, tartrate, and the like.

Methods for the preparation of solid pharmaceutical preparations are also well known in the art. Tablets may thus be prepared by mixing the active ingredient with ordinary adjuvants, fillers and diluents and subsequently compressing the mixture in a convenient tabletting machine. Examples of adjuvants, fillers and diluents comprise microcrystalline cellulose, corn starch, potato starch, lactose, mannitol, sorbitol talcum, magnesium stearate, gelatine, lactose, gums, and the like. Any other adjuvant or additive such as colourings, aroma, preservatives, etc. may also be used provided that they are compatible with the active ingredients.

In particular, the tablet formulations according to the invention may be prepared by direct compression of a compound of Formula I in admixture with conventional adjuvants or diluents. Alternatively, a wet granulate or a melt granulate of a compound of Formula I, optionally in admixture with conventional adjuvants or diluents may be used for compression of tablets.

Solutions of a compound of Formula I for injections may be prepared by dissolving the active ingredient and possible additives in a part of the solvent for injection, preferably sterile water, adjusting the solution to the desired volume, sterilisation of the solution and filling in suitable ampoules or vials. Any suitable additive conventionally used in the art may be added, such as tonicity agents, preservatives, antioxidants, solubilising agents, etc. Alternatively the active ingredient, e.g. as the free base, may be dissolved in a digestible or non-digestible oil, mixtures hereof or similar, to prepare an intramuscular depot formulation capable of releasing the active ingredient over a prolonged period of time.

Pharmaceutical formulations of the compound of Formula I to be used in transdermal applications, such as transdermal patches, may optionally contain permeation activators to facilitate the passage of the active ingredient through the skin.

In another aspect, the invention relates to a pharmaceutical composition comprising a therapeutically effective amount of a compound of Formula I, or a pharmaceutically acceptable acid addition salt thereof, and one or more pharmaceutically acceptable carriers, diluents and excipients.

### BRIEF DESCRIPTION OF THE FIGURES

FIG. 1: Dose-response curve for the concentration-dependent stimulation of intracellular Ca²⁺ release by dopamine in hD5-transfected CHO-Ga16 cells.

FIG. 2. X-ray of intermediate IXb. The absolute configuration was determined from the anomalous scattering of the ‘heavy’ chlorine atom.

### EXPERIMENTAL SECTION

Analytical LC/MS data were obtained on a PE Sciex API 150EX instrument equipped with atmospheric pressure photo ionisation and a Shimadzu LC-8A/SLC-10A LC system. Purity was determined by integration of the UV (254 nm) and ELSD traces. MS instruments are from PESciex (API), equipped with APPI-source and operated in positive ion mode. The retention times in the UV-trace (RT) are expressed in min. Solvents A was made of 0.05% TFA in water, while solvent B was made of 0.035% TFA and 5% water in acetonitrile. Several different methods have been used:

Method 14: API 150EX and Shimadzu LC8/SLC-10A LC system. Column: C-18 4.6×30 mm, 3.5 μm (Symmetry, Waters). Column temperature: rt. Gradient: reverse phase with ion pairing. Flow: 2 mL/min. Injection volume: 10 μL. Gradient: 10% B in A to 100% B over 4 min then 10% B in A for 1 min. Total run time: 5 min.

Method 20: API 150EX and Shimadzu LC8/SLC-10A LC system. Column: C-18 4.6×30 mm, 3.5 μm (Symmetry, Waters). Column temperature: 40° C. Flow: 2 mL/min. Injection volume: 15 μL. Gradient: reverse phase with ion pairing. Gradient: 10% B in A to 100% B over 4 min then 10% B in A for 1 min. Total run time: 5 min.

Method 25: API 150EX and Shimadzu LC10AD/SLC-10A LC system. Column: dC-18 4.6×30 mm, 3 μm (Atlantis, Waters). Column temperature: 40° C. Gradient: reverse phase with ion pairing. Flow: 3.3 mL/min. Injection volume: 15 μL. Gradient: 2% B in A to 100% B over 2.4 min then 2% B in A for 0.4 min. Total run time: 2.8 min.

Method 101: API 150EX and Shimadzu LC8/SLC-10A LC system. Column: C-18 4.6×30 mm, 3.51 μm (Symmetry, Waters). Column temperature: 60° C. Gradient, reverse phase with ion pairing. Flow: 3.3 mL/min. Injection volume: 15 μL. Gradient: 10% B in A to 100% B over 2.4 min then 10% B in A for 0.4 min. Total run time: 2.8 min.

Method 102: API 150EX and Shimadzu LC 8/SLC-10A LC system. Column: dC-18 4.6×30 mm, 3 μm (Atlantis, Waters). Column temperature: 40° C. Gradient, reverse phase with ion pairing. Flow: 3.3 mL/min. Injection volume: 15 μL. Gradient: 2% B in A to 100% B over 2.4 min then 2% B in A for 0.4 min. Total run time: 2.8 min.

Method 122: API 150EX and Shimadzu LC8/SLC-10A LC system. Column: Atlantis T3; 4.6×30 mm 3 μm (Waters). Column temperature: 40° C. Injection volume 10 micro-L. Gradient, reverse phase with ion pairing. Gradient: 2% B to 100% B in 2.4 min then 2% B in A for 0.4 min. Flow: 3.3 mL/min. Total run time 2.8 min.

Method 336: API 150EX and Shimadzu LC8/SLC-10A LC system. Column: Symmetry C18 3.5 μm, 4.6×30 mm (Waters). Column temperature 60° C. Injection volume 10 micro-L. Flow 4.3 mL/min. Gradient, reverse phase with ion pairing. Gradient 10% B in A for 2 min, 100% B for 0.10 min. total run time 2.1 min.

Method 344: API 150EX and Shimadzu LC8/SLC-10A LC system. Column: Symmetry C18 3.5 μm, 4.6×30 mm (Waters). Column temperature 60° C. Injection volume 5 micro-L. Flow rate 5.5 mL/min. Gradient, reverse phase with ion pairing. Gradient 10% B in A for 1.45 min, 100% B for 0.10 min. total run time 1.55 min.

Hydrogenation reactions were performed using either a standard Parr shaker or an Endavour instrument from Argonaut. In all cases, low pressure was used (1-5 bar hydrogen pressure).

The term “silica gel chromatography (EtOAc/heptane)” has the following meaning. The compound to be purified was usually dissolved in a small amount of DCM and loaded onto a column pre-packed with silica gel and eluted using a mixture of EtOAc and heptane, either in a isocratic fashion or with a gradient such as 0-100% of EtOAc in heptane. One example of a column loaded with silica gel used is “ISOLUTE SPE COLUMNS” [e.g. 20 g FLASH S±70 ml from International sorbent technology]. Alternatively, classical manual chromatographic purifications were performed using silica gel [e.g. Machery-Nagel 60 M; 0.04-0.063 mm, 230-400 mesh] with compound identification by standard TLC analysis performed on aluminium plates precoated with silica gel [e.g. Merck 60 F₂₅₄]. Compounds were visualized by illumination using a UV lamp (254 nm) or by charring after dipping in a solution of ammonium molybdate (6.25 g) and cerium(IV)sulfate (2.5 g) in 10% aqueous sulphuric acid (250 mL).

Microwave-accelerated reactions were performed in sealed microwave reactor vials. The experiments were performed on a Smith Synthesizer from Personal Chemistry.

The term “lyophilized” refers to the freeze-drying of a material using a Christ Aplha 2-4 LSC instrument from WWR International.

The terms “dried (Na₂SO₄)” and “dried (Mg₂SO₄)” refers to the removal of water from organic layers by the addition of dry Na₂SO₄ or Mg₂SO₄, respectively, followed by stirring for an appropriate amount of time to ensure an effective drying process. Then the solid is removed by filtration, and the filtrate is typically concentrated in vacuo (see below).

The term “concentrated in vacuo” has the following meaning. The volatiles were removed from the mixture using a standard rotary evaporator at reduced pressure. The term “dried in vacuo at 40° C.” refers to the use of a standard vacuum oven heated to 40° C. connected to an oil pump. The term “dried in vacuo” refers to a drying process in which the material to be dried is placed in a flask connected directly to an oil pump for a sufficient period of time to remove volatile components.

X-ray crystal structure determinations were performed as follows. The crystal of the compounds was cooled to 120 K using a Cryostream nitrogen gas cooler system. The data were collected on a Siemens SMART Platform diffractometer with a CCD area sensitive detector. The structures were solved by direct methods and refined by full-matrix least-squares against F² of all data. The hydrogen atoms in the structures could be found in the electron density difference maps. The non-hydrogen atoms were refined anisotropically. All the hydrogen atoms were at calculated positions using a riding model with O—H=0.84, C—H=0.99-1.00, N—H=0.92-0.93 Å. For all hydrogen atoms the thermal parameters were fixed [U(H)=1.2 U for attached atom]. The Flack x-parameters are in the range 0.0(1)-0.05(1), indicating that the absolute structures are correct. Programs used for data collection, data reduction and absorption were SMART, SAINT and SADABS [cf. “SMART and SAINT, Area Detector Control and Integration Software”, Version 5.054, Bruker Analytical X-Ray Instruments Inc., Madison, USA (1998), Sheldrick “SADABS, Program for Empirical Correction of Area Detector Data” Version 2.03, University of Gottingen, Germany (2001)]. The program SHELXTL [cf. Sheldrick “SHELXTL, Structure Determination Programs”, Version 6.12, Bruker Analytical X-Ray Instruments Inc., Madison, USA (2001)] was used to solve the structures and for molecular graphics.

### General Synthetic Methods for the Compounds of Formulas Ia and Ib

The procedures in connection with preparation of the compounds of Formula Ia and Ib are provided in the Schemes below.

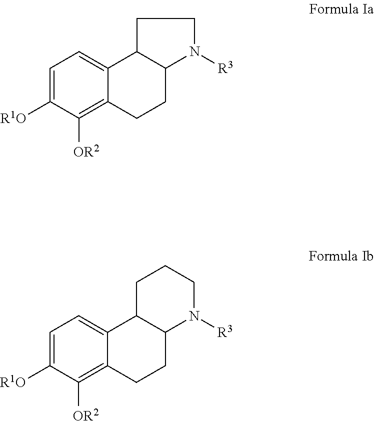

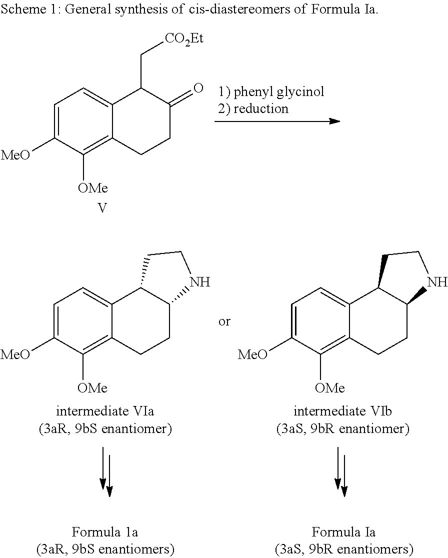

cis-Configured compounds of the Formula Ia can be prepared from keto-ester V (whose synthesis is described herein) by condensation with either enantiomer of phenyl glycinol followed by reduction under the conditions reported herein (for a closely related synthesis, see: M. D. Ennis, R. L. Hoffman, N. B. Ghazal, D. W. Old, P. A. Mooney J. Org. Chem. 1996, 61, 5813). The choice of the enantiomer of phenyl glycinol dictates whether the reaction delivers intermediate VIa or intermediate VIb when using R-(−)-phenyl glycinol or S-(+)-phenyl glycinol, respectively. Subsequent alkylation, reductive amination, or a two-step acylation/reduction sequence installs the R₃ group. Cleavage of the two aromatic methyl ethers with BBr₃ or 48% aqueous HBr delivers the compounds of Formula Ia in which R₁═R₂═H. These catechol amines can be reacted with ClCH₂Br or a similar reagent in the presence of base (e.g. cesium carbonate under the conditions described herein for the synthesis of example 1a9) to give the compounds of Formula Ia in which R₁ and R₂ are joined to form a CH₂-group. The catechol amines can be reacted with acid chlorides in trifluoroacetic acid to give the compounds of Formula Ia in which R₁ and R₂ are esters.

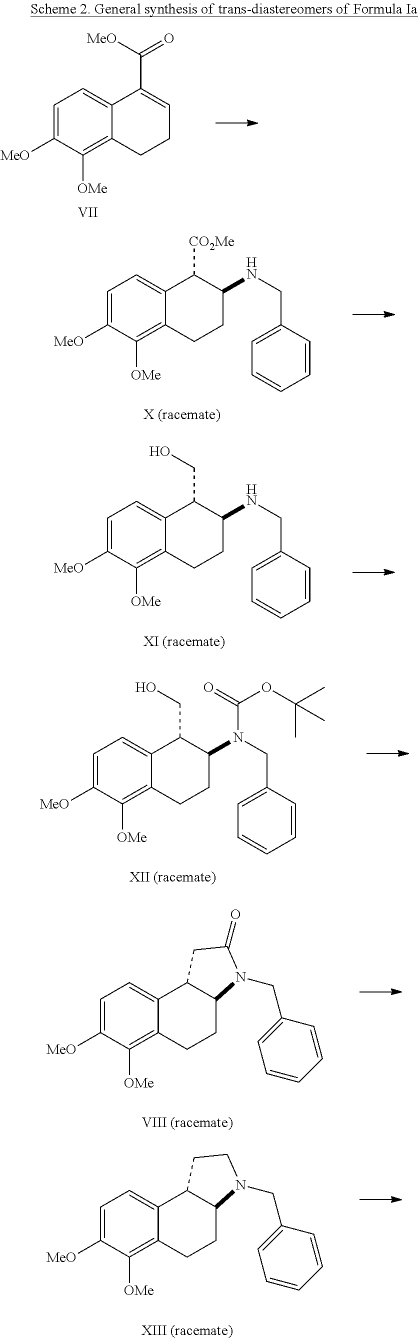

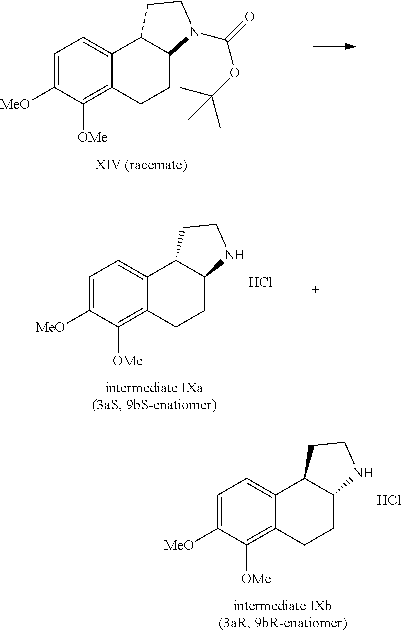

trans-Configured compounds of formula Ia can be prepared from unsaturated ester VII (the synthesis of which is described herein) by the conjugate addition of benzylamine. Subsequent reduction with lithium aluminiumhydride, N-Boc protection, and reaction with acetone cyanohydrin in the presence of triphenylphosphine and diethyl azodicarboxylate (DEAD) followed by treatment with acid and base delivers intermediate VIII. This material is reduced with lithium aluminiumhydride and subsequently by hydrogen over palladium-on-charcoal in the presence of Boc₂O. The resulting material can be resolved by chiral chromatography under the conditions described herein to provide intermediate IXa and intermediate IXb as their hydrochloride salts after removal of the Boc-group. From these compounds, subsequent alkylation, reductive amination, or a two-step acylation/reduction sequence installs the R₃ group. Cleavage of the two aromatic methyl ethers with BBr₃ or 48% aqueous HBr delivers the compounds of the invention formula Ia in which R₁═R₂═H. These catechol amines can be reacted with ClCH₂Br or a similar reagent in the presence of base (e.g. cesium carbonate under the conditions described herein for the synthesis of example 1a9) to give the compounds of formula Ia in which R₁ and R₂ are joined to form a CH₂-group. The catechol amines can be reacted with acid chlorides in trifluoroacetic acid to give the compounds of the formula Ia in which R₁ and R₂ are esters.

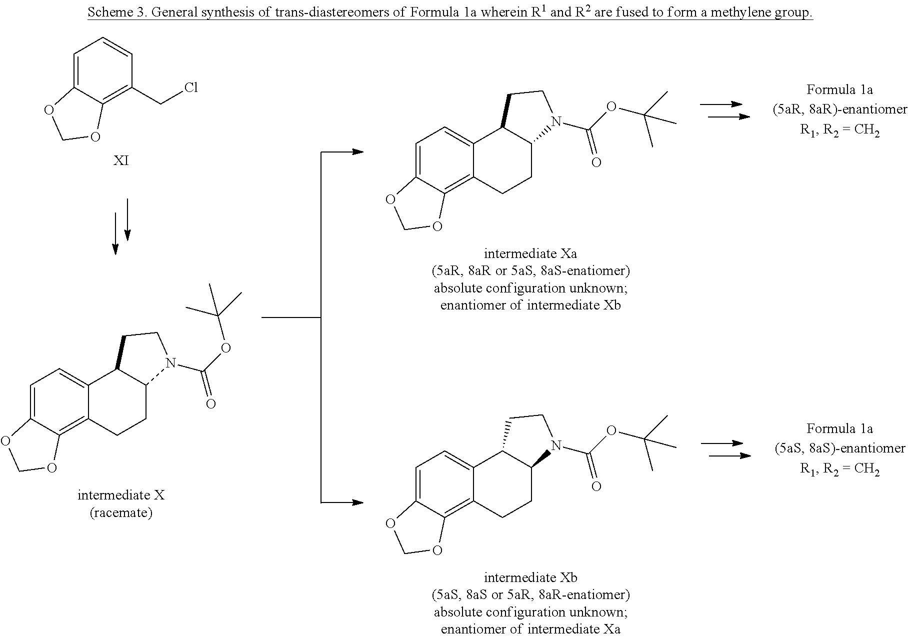

Compounds of Formula Ia in which R¹ and R² are fused to form a methylene group can be prepared from intermediates Xa and Xb (or vice-versa). Resolution of intermediate X by chiral chromatography for example under the conditions described herein gives access to these two compounds. Intermediate X itself can be prepared from XI (whose synthesis is described in the literature: Z. Kiparissides, R. H. Fichtner, J. Poplawski, B. C Nalliah, D. B. MacLean Can. J. Chem. 1980, 58, 2770) over a series of steps as described herein.

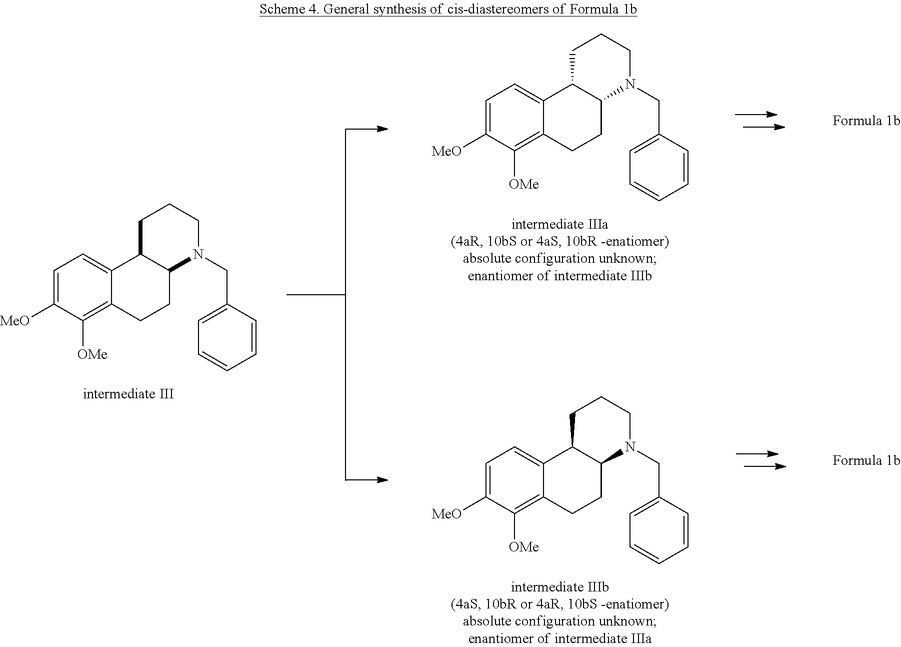

Compounds of the Formula 1b can be obtained from cis amine III (c.f. J. G. Cannon, C. Suarez-Gutierrez, T. Lee J. Med. Chem. 1979, 22, 341) by initial chiral chromatography, for example under the conditions described herein. Subsequent removal of the benzyl group followed by alkylation, reductive amination, or by a two-step acylation/reduction sequence installs the R₃ group. The resulting tertiary amines can be resolved by chiral chromatography or by classic resolution techniques. Cleavage of the two aromatic methyl ethers with BBr₃ or 48% aqueous HBr delivers the compounds of Formula 1b in which R₁═R₂═H. These catechol amines can be reacted with ClCH₂Br or a similar reagent in the presence of base (e.g. cesium carbonate under the conditions described herein for the synthesis of example 1a9) to give the compounds of Formula 1b in which R₁ and R₂ are fused to form a CH₂-group. The catechol amines can be reacted with acid chlorides in trifluoroacetic acid to give the compounds of Formula 1b in which R₁ and R₂ are esters.

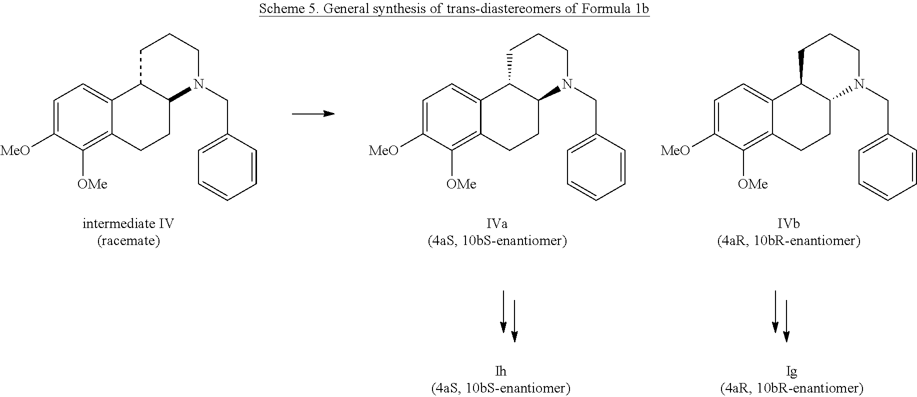

Compounds of Formula 1b can be obtained from trans amine IV (c.f. J. G. Cannon, C. Suarez-Gutierrez, T. Lee J. Med. Chem. 1979, 22, 341) by chiral chromatography under the conditions described herein to give intermediates IVa and IVb. The benzyl group can be removed by hydrogenolysis and the R₃-group can be installed by alkylation, reductive amination, or by a two-step acylation/reduction sequence. Cleavage of the two aromatic methyl ethers with BBr₃ or 48% aqueous HBr delivers the compounds of Formula 1b in which R₁═R₂═H. These catechol amines can be reacted with ClCH₂Br or a similar reagent in the presence of base (e.g. cesium carbonate under the conditions described herein for the synthesis of example 1a9) to give the compounds of Formula 1b in which R₁ and R₂ are joined to form a CH₂-group. The catechol amines can be reacted with acid chlorides in trifluoroacetic acid to give the compounds of Formula 1b in which R₁ and R₂ are esters.

### Preparation of Intermediates

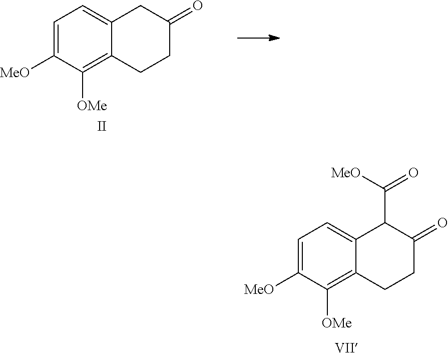

Tetralone II (98 g) and sodium methoxide (23.5 g) were refluxed in a mixture of dimethyl carbonate (1600 mL) and methanol (260 mL) for 2 hours. The volatiles were removed in vacuo, and the residual solid was washed with methanol to afford keto ester VII′ (69 g).

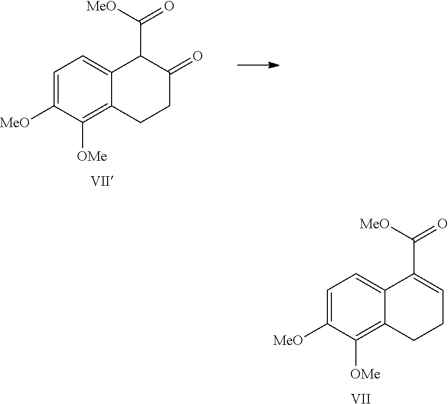

Keto ester VII′ (63 g) was treated with sodium borohydride (10.2 g) in a mixture of tetrahydrofuran (500 mL) and water (50 mL) at room temperature for 1 hour. The volatiles were removed in vacuo. The residue was treated with mesyl chloride (18 mL) in pyridine (200 mL) at room temperature overnight. The volatiles were removed in vacuo to afford unsaturated ester VII (49 g) after an extractive work-up.

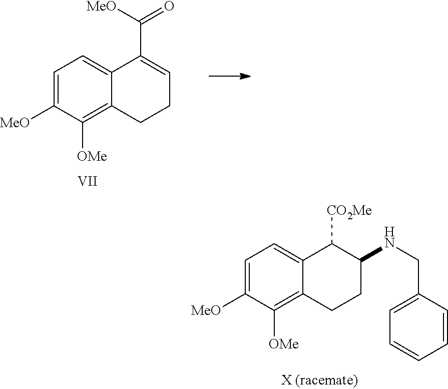

Unsaturated ester VII (6.2 g) was dissolved in benzyl amine (8.3 mL), and Triton-B (benzyltrimethylammonium hydroxide; 4 drops) was added. The resulting mixture was stirred at room temperature for 70 hours. The resulting slurry was stirred with water (50 mL), the water was decanted off, and this procedure was repeated twice to afford a beige semi-solid. This material was triturated with heptane (40 mL) and collected by filtration to afford compound X as a white solid (3.3 g).

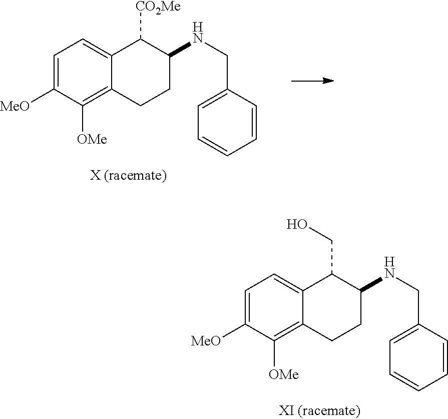

Compound X (10.0 g) was dissolved in tetrahydrofuran (50 mL), and lithium aluminiumhydride (1M in tetrahydrofuran; 40 mL) was added drop-wise. After 1 hour, the reaction was quenched with 15% aqueous sodium hydroxide (ca 6 mL), and filtered. The filtrate was concentrated in vacuo, and re-dissolved in methylene chloride (200 mL). This solution was washed with 5% aqueous sodium carbonate (20 mL), dried over sodium sulfate, filtered, and concentrated in vacuo to afford compound XI as a yellow oil (9.1 g).

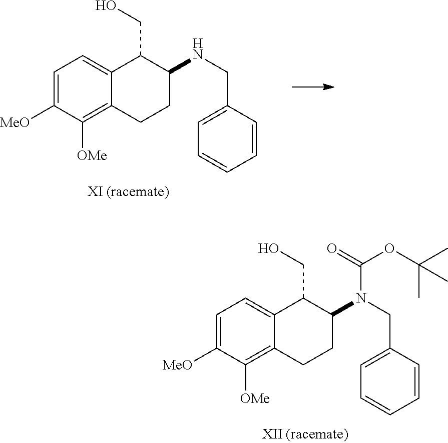

Compound XI (8.0 g) was suspended in water (50 mL) and treated with Boc₂O (6.2 g) under vigorous stirring for 4 hours. The crude mixture was extracted with diethyl ether (2×100 mL). The combined organic layers were dried over sodium sulfate, filtered, and concentrated in vacuo. The residue was purified by chromatography (eluent: ethyl acetate/heptanes 1:2) to afford compound XII as a white foam (5.1 g).

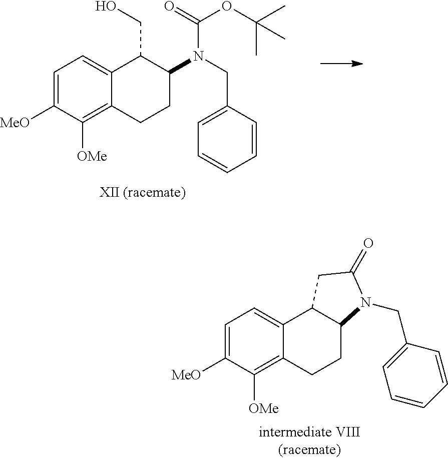

Compound XII (3.5 g) and triphenyl phosphine were dissolved in diethyl ether, and the solution was cooled to 0° C. DEAD (2.2 M in toluene; 7.4 mL) was added at such a rate that the temperature was maintained below 5° C. An additional 80 mL of diethyl ether was added followed by acetone cyanohydrin (1.5 mL). The mixture was allowed to warm to room temperature overnight. Next morning, the crude mixture was concentrated in vacuo, and the residue was treated with 37% aqueous HCl (15 mL) for 20 min. The mixture was washed with diethyl ether (5×50 mL). The aqueous layer was diluted with methanol (30 mL) and 27% aqueous sodium hydroxide (until pH 12-13). The resulting mixture was refluxed overnight. Next morning pH was adjusted to 7, and the product was extracted into methylene chloride (2×60 mL). The combined organic extracts were dried over sodium sulfate, filtered, and concentrated in vacuo. The residue was purified by chromatography (eluent: ethyl acetate/heptanes 1:1) afford intermediate VIII (1.3 g).

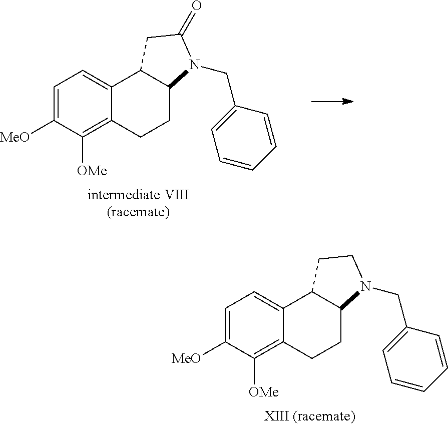

Intermediate VIII (1.3 g) was dissolved in tetrahydrofuran (50 mL). lithium aluminiumhydride (1 M in tetrahydrofuran; 4 mL) was added, and the mixture was refluxed for 1 hour. Water (0.5 mL) was added to quench the reaction. The solid was filtered off, and the filtrate was concentrated in vacuo. The residue was dissolved in methylene chloride (60 mL) and washed with 5% aqueous sodium carbonate (10 mL). The organic layer was dried over sodium sulfate, filtered, and concentrated in vacuo to afford a solid. This material was re-crystallized from methanol to afford compound XIII (0.96 g).

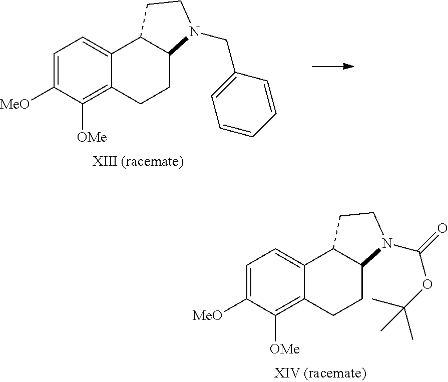

Compound XIII (12.0 g) was suspended in ethyl acetate (200 mL). Boc₂O (8.3 g) was added followed by 10% Pd/C (0.5 g). The resulting mixture was treated with H₂ (3 bar) for 2 days. The catalyst was filtered off, and the filtrate was concentrated in vacuo. The residue was dissolved in methylene chloride (200 mL) and washed with 2% aqueous citric acid (50 mL). The organic layer was dried over sodium sulfate, filtered, and concentrated in vacuo to afford compound XIV as a white solid (12.3 g).

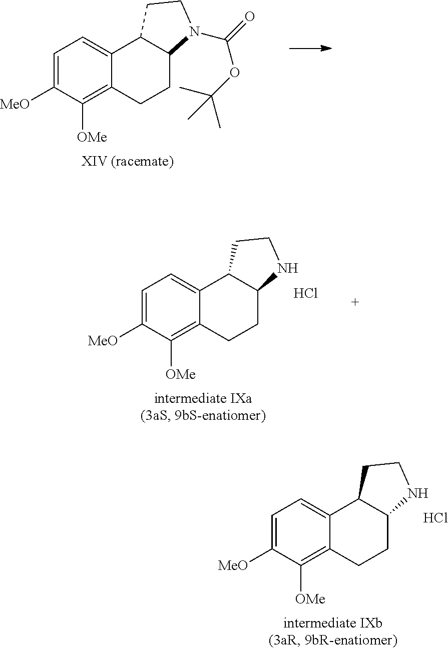

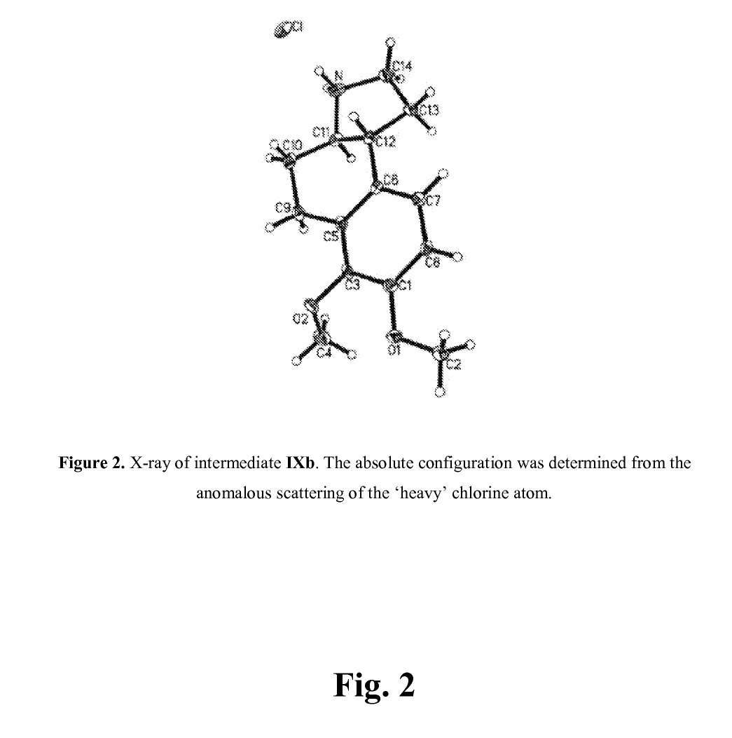

Compound XIV (12.3 g) was resolved by chiral SFC using stacked injection (0.4 mL per run) onto a Chiral Pak AD-H 250×21.2 mm 5 micro-m column with a solution of 0.1% diethyl amine in ethanol as modifier. The concentration of the modifier was 40% and the flow rate was 50 mL/minute. 50 mL/minute. The column temperature was 35° C. and the pressure was 100 bar. The two enantiomers were treated with HCl in methanol and concentrated in vacuo to afford intermediate IXa (4.1 g) and intermediate IXb (3.9 g) as white solids. The absolute configuration was determined by X-ray crystallography of intermediate IXb (c.f. FIG. 2).

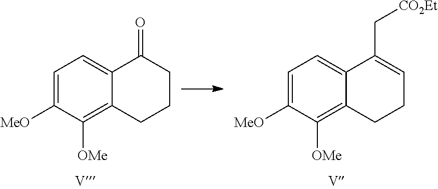

Sodium metal (16 g) was added to abs. ethanol (600 mL) under a nitrogen atmosphere. To the resulting solution was added dry triethyl phosphonoacetate (157 g). After stirring for 10 min tetralone V′″ (for a synthesis of this material see for example: T. Beetz, D. G Meuleman, J. H. Wieringa J. Med. Chem. 1982, 25, 714) (120 g) was added over 10 min and the mixture was stirred for 2.5 h at 80° C. The reaction mixture was cooled to room temperature, diluted with water (1500 mL) and extracted with ethyl acetate. The organic phases were washed with water and dried over sodium sulfate, the solvent was evaporated in vacuo. The residue was purified by chromatography (eluent: petroleum ether/ethyl acetate=15:1) to afford unsaturated ester V″ (50 g).

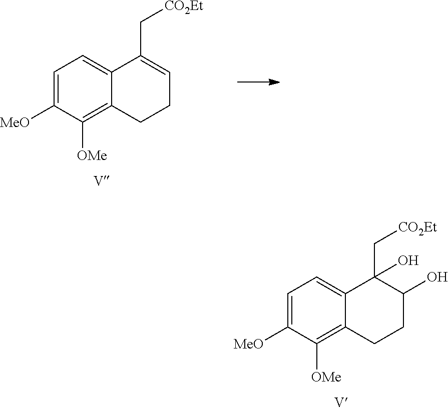

Unsaturated ester V″ (27.6 g) was dissolved in acetone/tert-butyl alcohol/water (150:40:50 mL) and NMO (N-methylmorpholine-N-oxide; 12.9 g) was added. A solution of OsO₄ (0.08M in tert-butyl alcohol; 5.3 mL) was added. The resulting mixture was stirred at room temperature for 2 hours before it was stirred at ca 55° C. for 1 hour and then for 1 hour at room temperature. The solvents were removed in vacuo, and the residue was dissolved in ethyl acetate (500 mL). This solution was washed with 5% aqueous sodium hydrideSO₃, saturated aqueous sodium carbonate, dried over sodium sulfate, filtered, and concentrated in vacuo to afford diol V′ as an orange oil that solidified on standing (23.6 g).

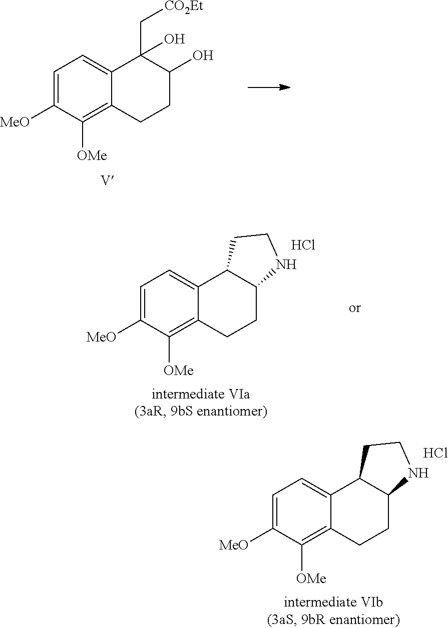

In situ preparation of keto-ester V: diol V′ (12.5 g) was dissolved in diethyl ether (500 mL) and treated with BF₃-diethyl ether (5 mL) at room temperature for 1 hour. The crude mixture was washed with water and saturated aqueous sodium carbonate, dried over sodium sulfate, filtered, and concentrated in vacuo. The resulting yellow oil V (11 g) was refluxed overnight in toluene (500 mL) in the presence of R-(−)-phenyl glycinol (6.1 g) using a Dean-Start trap. The toluene was removed by concentration in vacuo, and the residue was purified by chromatography (eluent: ethyl acetate/heptanes 0:1 to 1:1) to afford an oil (9.8 g). This material was dissolved in tetrahydrofuran (150 mL) and reacted with borane (1M in tetrahydrofuran) at −75° C. for 1 hour. The suspension was allowed to warm to room temperature, stirred at room temperature for 1 hour, then refluxed for 1 hour, before the reaction was quenched with methanol. The resulting mixture was concentrated in vacuo, and the residue was treated with 6M aqueous HCl (100 mL) for 2 hours. The mixture was basified and extracted with diethyl ether. The organic layer was dried over sodium sulfate, filtered, and concentrated in vacuo to afford a yellow oil (9 g). This material was dissolved in methanol (250 mL). Ammonium formate (8 g) was added followed by 10% Pd/C (1 g). The resulting mixture was stirred for 24 hours at room temperature, before it was filtered and concentrated in vacuo. The residue was dissolved in diethyl ether, washed with 2M aqueous sodium hydroxide, dried over sodium sulfate, filtered, and treated with HCl gas. Most of the solvent was removed in vacuo, and acetonitrile (40 mL) was added to precipitate intermediate VIa as a white solid (3.1 g). In a similar manner, diol V′ (24.8 g) was converted to intermediate VIb (3.8 g) by the use of S-(+)-phenyl glycinol. The absolute configuration of intermediates VIa and VIb was tentatively assigned based on analogy to the literature (c.f. M. D. Ennis, R. L. Hoffman, N. B. Ghazal, D. W. Old, P. A. Mooney J. Org. Chem. 1996, 61, 5813).

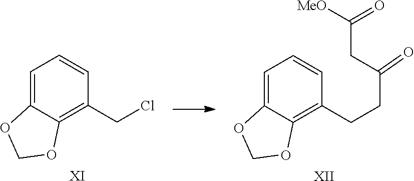

To a suspension of sodium hydride (60% oil dispersion; 2.5 g) in tetrahydrofuran (100 mL) was added drop-wise 3-oxo-butyric acid methyl ester (7 g) at −50° C. The resulting mixture was stirred at 0° C. for 15 minutes and cooled to −50° C. (n-Bu)Li (5M in hexane; 12 mL) was added drop-wise, and the mixture was stirred at −50° C. for 0.5 hours. A solution of compound XI (10 g) in tetrahydrofuran (100 mL) was added drop-wise, and the mixture was allowed to warm to room temperature overnight. The mixture was cooled to 0° C. and quenched with acetic acid (20 mL). The crude mixture was partitioned between ethyl acetate and water. The organic layer was washed with saturated aqueous sodium chloride, dried over magnesium sulfate, filtered, and concentrated in vacuo. The residue was purified by chromatography (eluent: petroleum ether/ethyl acetate 15:1) to afford keto ester XII (8 g).

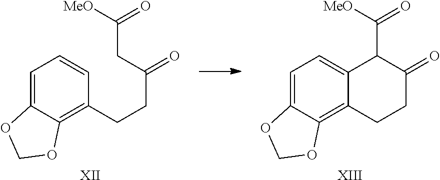

To a mixture of 4-methyl-benzenesulfonyl azide (7 g) and keto ester XII (7 g) in acetonitrile (100 mL) was added drop-wise triethyl amine (5 mL). The resulting mixture was stirred overnight at room temperature. The crude mixture was partitioned between ethyl acetate and water. The organic layer was washed with saturated aqueous sodium chloride, dried over magnesium sulfate, filtered, and concentrated in vacuo. The residue was purified by chromatography (eluent: petroleum ether/ethyl acetate 10:1) to afford the intermediate azo-compound. (7 g). 3 g of this material was dissolved in methylene chloride (50 mL) and mixed with a solution of rhodium(II)acetate (10 mg) in methylene chloride (50 mL). The resulting mixture was refluxed for 1 hour before trifluoroacetic acid (0.1 mL) was added, and the mixture was heated for an additional 1 hour. The crude mixture was diluted with water. The organic layer was washed with saturated aqueous sodium chloride, dried over magnesium sulfate, filtered, and concentrated in vacuo. The residue was purified by chromatography (eluent: petroleum ether/ethyl acetate 20:1) to afford the cyclized product XIII (1.2 g).

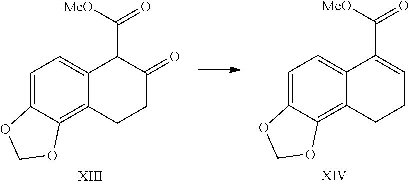

Compound XIII (38 g) was dissolved in tetrahydrofuran (250 mL) and treated with sodium borohydride (7 g) at 0° C.→room temperature. After 1 hour at room temperature, the reaction was quenched with water. The desired intermediate alcohol was extracted into methylene chloride. The organic extract was dried over sodium sulfate, filtered, and concentrated in vacuo. The residue was dissolved in pyridine (300 mL) and reacted with methane sulfonyl chloride (11 mL) at room temperature overnight. The crude mixture was partitioned between methylene chloride and water. The organic layer was washed with saturated aqueous sodium chloride, dried over magnesium sulfate, filtered, and concentrated in vacuo. The residue was purified by precipitation from ethyl acetate to afford unsaturated ester XIV (26.2 g) as a white

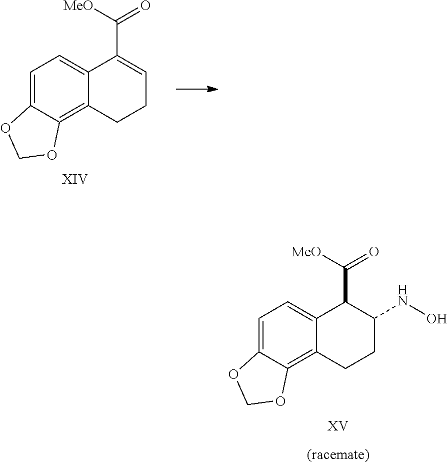

Compound XIV (13.9 g) and hydroxylamine hydrochloride (13.9 g) were dissolved in methanol (150 mL). potassium carbonate (27.6 g) was added, and the mixture was refluxed for 2 hours and stirred at room temperature for 1 hour. Additional hydroxylamine hydrochloride (4.0 g) and potassium carbonate (8.0 g) were added, and the mixture was stirred at room temperature overnight. The precipitated compound XV was filtered off as a white solid (12.1 g).

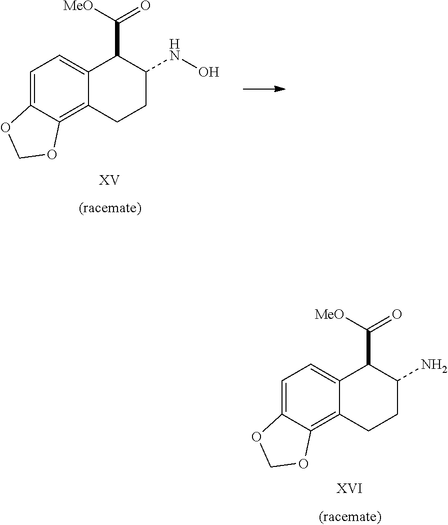

Compound XV (9.5 g) was dissolved in acetic acid (100 mL) and treated with zinc (4.7 g) at 65° C. overnight. The precipitated solid was removed by filtration, and the filtrate was concentrated in vacuo. The residue was partitioned between water and ethyl acetate. sodium hydroxide was added until pH ˜10. The organic layer was dried over magnesium sulfate, filtered and co-concentrated with silica gel. Chromatography (eluent: heptane/ethyl acetate 1:1→ethyl acetate/methanol/triethyl amine 85:10:5) gave amine XVI (7.0 g).

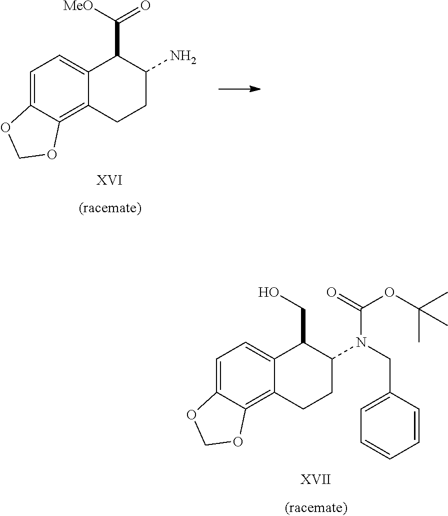

Compound XVI (8.6 g) was dissolved in a mixture of tetrahydrofuran (75 mL) and 2M aqueous potassium carbonate (50 mL) and reacted with benzoyl chloride (4.4 mL) at room temperature for 15 minutes. The organic layer was concentrated in vacuo, and the residual solid was washed with diethyl ether to afford the intermediate amino alcohol as a white solid. This material was dissolve in tetrahydrofuran (200 mL) and reacted with lithium aluminiumhydride (2M in tetrahydrofuran; 75 mL) at 70° C. overnight. Additional lithium aluminiumhydride (2M in tetrahydrofuran; 7.5 mL) was added, and the mixture was refluxed for 4 hours. The reaction was quenched with 10% aqueous sodium hydroxide (6 mL). The precipitated solid was filtered off, and the filtrate was concentrated in vacuo to afford the intermediate amino alcohol. 8.4 g of this material was dissolved in tetrahydrofuran (50 mL) and potassium carbonate (4.4 g) in water (25 mL) was added followed by Boc₂O (7.1 g). The mixture was stirred at room temperature for 6 hours. diethyl ether was added, and the aqueous layer was extracted twice with diethyl ether. The combined organic layers were washed with saturated aqueous sodium chloride, dried over magnesium sulfate, filtered, and concentrated in vacuo to afford compound XVII (11.2 g) as a yellow oil.

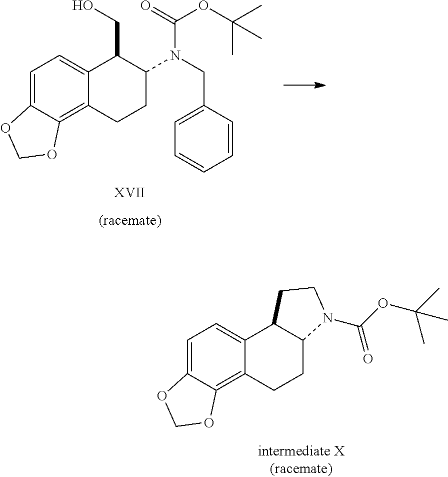

A solution of compound XVII (11.2 g) and PPh₃ (14.2 g) in diethyl ether (200 mL) was cooled to −10-−15° C. and di-iso-propyl azodicarboxylate (11 mL) was added drop-wise. To this mixture was added acetone cyanohydrin (13.8 mL). The cooling bath was removed, and the mixture was stirred at room temperature for 3 hours. The volatiles were removed in vacuo, and the residue was filtered through a plough of silica gel (eluent: heptane/ethyl acetate 10:1) to afford the intermediate Boc-protected amino nitrile. This material was refluxed in a mixture of diethyl ether (200 mL) and HCl in diethyl ether (2M; 27 mL) for 0.5 hours. The volatiles were removed in vacuo, and the residue was suspended in methanol (100 mL). HCl gas was bubbled through the mixture for 30 seconds, and the resulting mixture was stirred overnight at room temperature. The volatiles were removed in vacuo, and the residue was suspended in ethanol (200 mL) and refluxed with 27% aqueous sodium hydroxide (25 mL) for 8 hours, before it was stirred overnight at room temperature. The crude mixture was cooled on an ice/water bath and pH was adjusted to ˜6 with 37% aqueous HCl. The ethanol was removed in vacuo, and the aqueous residue was extracted with methylene chloride (2×100 mL). The combined organic extracts were dried over magnesium sulfate, filtered, and concentrated in vacuo to afford a brown foam. This material was purified by chromatography (eluent: ethyl acetate/heptane 1:2) to afford the intermediate lactam as a yellow solid. This material was dissolved in tetrahydrofuran (25 mL) and refluxed with lithium aluminium hydride (1M in tetrahydrofuran, 10 mL) for 1 hour. Excess lithium aluminium hydride was quenched with 2M aqueous sodium hydroxide (0.5 mL) and diluted with tetrahydrofuran (200 mL). The suspension was filtered, and the filtrate was concentrated in vacuo. The residue was dissolved in methylene chloride and washed with water and saturated aqueous sodium chloride. The organic layer was dried over magnesium sulfate, filtered, and concentrated in vacuo. The residue and 10% Pd/C (250 mg) were suspended in ethyl acetate/ethanol (50 mL/10 mL) and treated with hydrogen gas (3 bar) at room temperature overnight. Next morning, HCl gas was bubbled through the mixture for 30 seconds, and the resulting mixture was treated with hydrogen gas (3 bar) at room temperature overnight. The catalyst was filtered off, and the filtrate was concentrated in vacuo. The residue was dissolved in tetrahydrofuran (25 mL) and treated with potassium carbonate (1.2 g in 5 mL water) and Boc₂O (1.6 g) at room temperature overnight. diethyl ether and a little water were added, and the organic layer was washed with 2% aqueous citric acid and saturated aqueous sodium chloride, before it was dried over magnesium sulfate, filtered, and concentrated in vacuo to afford intermediate X (950 mg) as a white solid.

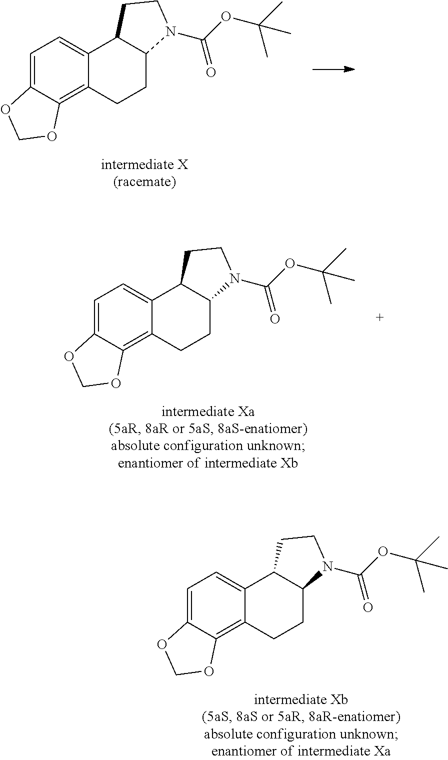

Intermediate X (1.5 g dissolved in 60 mL acetonitrile) was resolved by chiral SFC using stacked injection (0.4 mL per run) onto a Chiral Pak AD-4 250×21.2 mm 5 micro-m column with a solution of 0.1% diethyl amine in ethanol as modifier. The concentration of the modifier was 40% and the flow rate was 50 mL/minute. The column temperature was 35° C. and the pressure was 100 bar. This gave intermediates Xa and intermediate Xb as white solids.

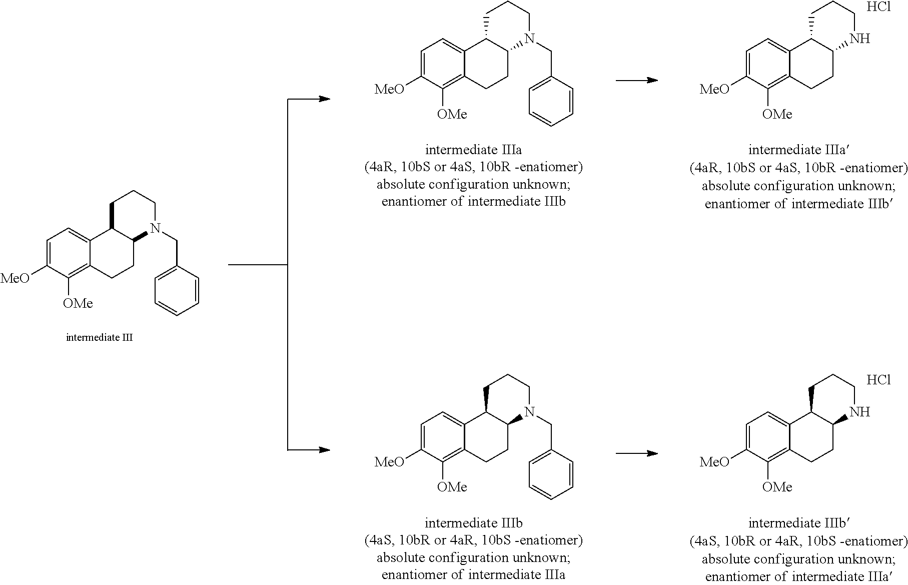

Intermediate III (3.37 g dissolved in 75 mL acetonitrile) was resolved by chiral SFC using stacked injection (0.4 mL per run) onto a OJ-H 250×21.2 mm 5 micro-m column with a solution of 0.1% diethyl amine in ethanol as modifier. The concentration of the modifier was 20% and the flow rate was 50 mL/minute. The column temperature was 35° C. and the pressure was 100 bar. This gave intermediate IIIa (1.83 g; first eluting enantiomer) and intermediate IIIb (1.45 g, second eluting enantiomer) as white solids. Intermediate IIIa (0.5 g) was treated with 10% Pd/C (100 mg) and hydrogen gas (3 bar) overnight in a mixture of 37% aqueous HCl (1 mL), chloroform (5 mL), and ethanol (25 mL) overnight at room temperature. The catalyst was filtered off, and the filtrate was concentrated in vacuo. The residue was subjected to the same reaction and purification conditions again. The resulting material was partitioned between ethyl acetate (25 mL) and 2 M aqueous sodium hydroxide (2×25 mL). The organic layer was dried over magnesium sulfate, filtered, and concentrated in vacuo to afford intermediate IIIa′ (302 mg) as a white solid. Intermediate IIIb (0.87 g) was treated with 10% Pd/C (100 mg) and hydrogen gas (3 bar) overnight in a mixture of 37% aqueous HCl (1 mL), ethanol (25 mL), and methylene chloride (10 mL). The catalyst was filtered off, and the filtrate was concentrated in vacuo to afford intermediate IIIb′ (0.48 g) as a white solid (some material lost during the hydrogenation reaction).

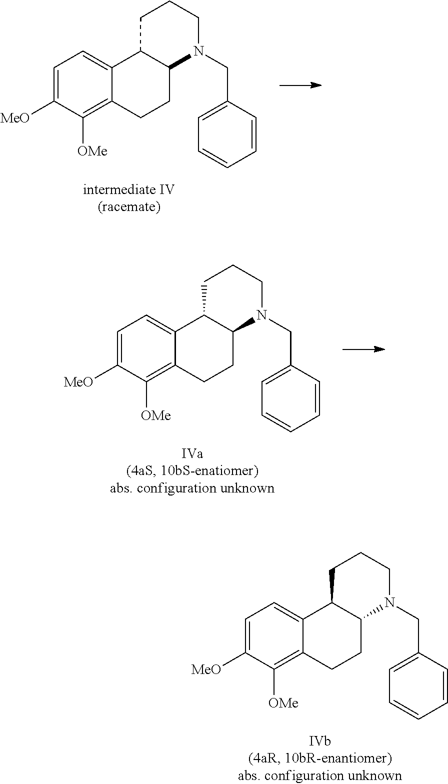

Intermediate IV (50 g) was resolved by chiral SFC using stacked injection (0.4 mL per run) onto a Chiralpack AD 250×21.2 mm 5 micro-m column with a solution of 0.2% diethyl amine in ethanol as modifier. The concentration of the modifier was 25% and the flow rate was 50 mL/minute. The column was held at room temperature and the pressure was 200 bar. This gave intermediate IVa (9.7 g; first eluting enantiomer) and intermediate IVb (22.1 g, second eluting enantiomer) as white solids.

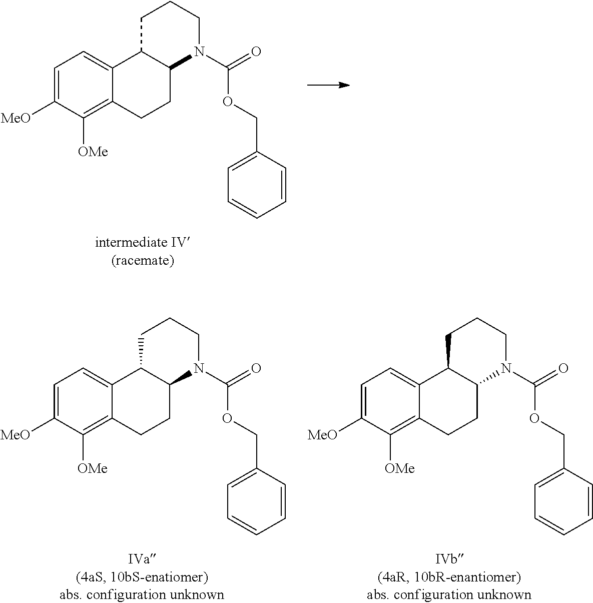

Alternatively, intermediate IV (1.61 g) was debenzylated by treatment with hydrogen gas (3 bar) in the presence of 10% Pd/C (0.5 g) in a mixture of 37% aqueous HCl (1 mL), methylene chloride (20 mL), and ethanol (80 mL) at room temperature overnight. The catalyst was filtered off, and the filtrate was concentrated in vacuo. The residue was precipitated from ethyl acetate to afford a solid. 0.15 g of this material was treated with KHCO₃ (0.37 g) and benzyl chloroformate (0.083 mL) in a mixture of methylene chloride (20 mL) and water (20 mL) over 1 hour at 0° C. room temperature. The organic layer was washed with water, dried over potassium carbonate, filtered, and concentrated in vacuo to afford 0.12 g of intermediate IV′. Intermediate IV′ (0.88 g) was resolved by chiral SFC using stacked injection (0.4 mL per run) onto a Chiralpack AD 250×4.6 mm 5 micro-m column with ethanol as modifier. The concentration of the modifier was 30% and the flow rate was 50 mL/minute. The column temperature was 25° C. and the pressure was 200 bar. This gave intermediate IVa″ (0.18 g; second eluting enantiomer) and intermediate IVb″ (0.17 g, first eluting enantiomer) as white solids.

### Compounds of the Invention

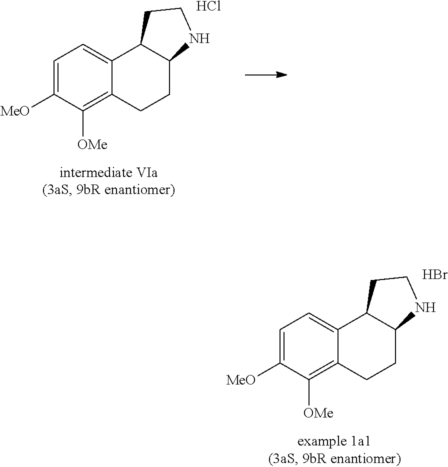

Example 1a1 (3aS,9bR)-2,3,3a,4,5,9b-Hexahydro-1H-benzo[e]indole-6,7-diol hydrobromide. Intermediate VIa (1 mmol) was treated with 48% aqueous HBr (2 mL) under microwave conditions at 120° C. for 30 min. After cooling to room temperature, the precipitated solid was collected by filtration and dried to afford example 1a1 (100 mg) as a white solid. LC/MS (method 122): RT (UV)=0.53 min, UV-purity 79.0%, ELS-purity 100%, mass observed 206.2.

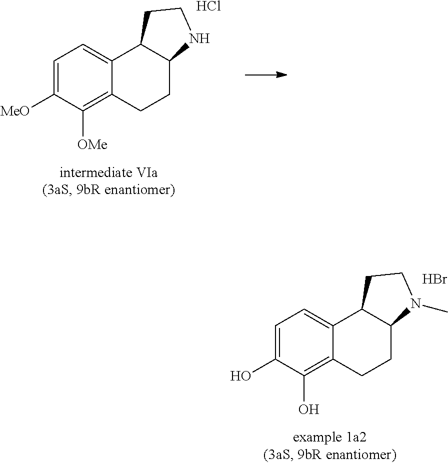

Example 1a2 (3aS,9bR)-3-Methyl-2,3,3a,4,5,9b-hexahydro-1H-benzo[e]indole-6,7-diol hydrobromide. Intermediate VIa (250 mg) was treated overnight at room temperature with formaldehyde (13.4M in water, 100 microL) and sodium cyanoborohydride (116 mg) in methanol (10 mL). The volatiles were removed in vacuo, and the residue was purified by chromatography (eluent: heptane/ethyl acetate/triethyl amine 10:10:1) to afford 200 mg of an intermediate. This material was reacted with 48% aqueous HBr (2 mL) under microwave conditions at 120° C. for 30 min. After cooling to room temperature, the precipitated solid was collected by filtration and dried to afford example 1a2 (25 mg) as a white solid. LC/MS (method 122): RT (UV)=0.54 min, UV-purity 84.3%, ELS-purity 74.7%, mass observed 220.4.

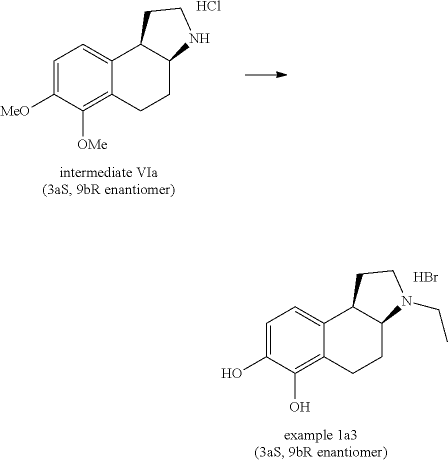

Example 1a3 (3aS,9bR)-3-Ethyl-2,3,3a,4,5,9b-hexahydro-1H-benzo[e]indole-6,7-diol hydrobromide. Intermediate VIa (300 mg) was treated overnight at room temperature with ethyl iodide (181 mg) and potassium carbonate (153 mg) in acetonitrile (10 mL). The volatiles were removed in vacuo, and the residue was purified by chromatography (eluent: heptane/ethyl acetate/triethyl amine 10:10:1) to afford 170 mg of an intermediate. This material was reacted with 48% aqueous HBr (2 mL) under microwave conditions at 120° C. for 30 min. After cooling to room temperature, the precipitated solid was collected by filtration and dried to afford example I a3 (63 mg) as a white solid. LC/MS (method 122): RT (UV)=0.60 min, UV-purity 82.9%, ELS-purity 95%, mass observed 234.1.

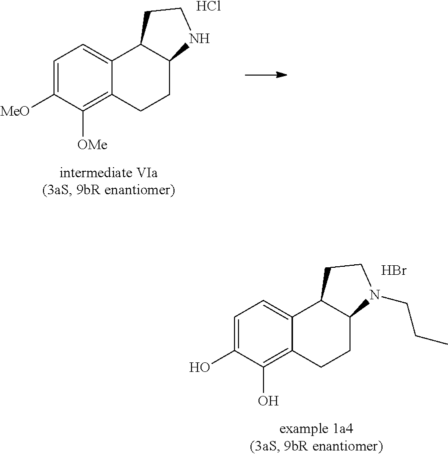

Example 1a4 (3aS,9bR)-3-n-Propyl-2,3,3a,4,5,9b-hexahydro-1H-benzo[e]indole-6,7-diol hydrobromide. Intermediate VIa (300 mg) was treated overnight at room temperature with n-propyl bromide (181 mg) and potassium carbonate (153 mg) in acetonitrile (10 mL). The volatiles were removed in vacuo, and the residue was purified by chromatography (eluent: heptane/ethyl acetate/triethyl amine 10:10:1) to afford 230 mg of an intermediate. This material was reacted with 48% aqueous HBr (2 mL) under microwave conditions at 120° C. for 30 min. After cooling to room temperature, the precipitated solid was collected by filtration and dried to afford example I a4 (117 mg) as a white solid. LC/MS (method 122): RT (UV)=0.70 min, UV-purity 81.6%, ELS-purity 100%, mass observed 248.5.

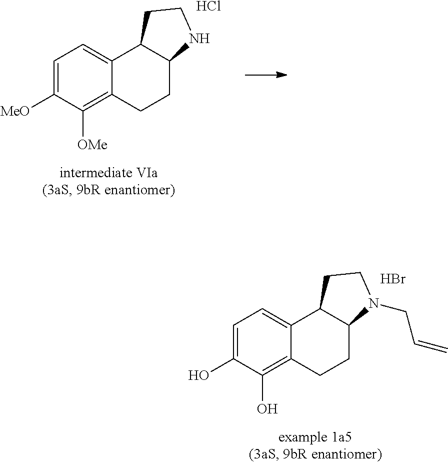

Example 1a5 (3aS,9bR)-3-Allyl-2,3,3a,4,5,9b-hexahydro-1H-benzo[e]indole-6,7-diol hydrobromide. Intermediate VIa (300 mg) was treated overnight at room temperature with allyl bromide (140 mg) and potassium carbonate (153 mg) in acetonitrile (10 mL). The volatiles were removed in vacuo, and the residue was purified by chromatography (eluent: heptane/ethyl acetate/triethyl amine 10:10:1) to afford 190 mg of an intermediate. This material was reacted with 48% aqueous HBr (2 mL) under microwave conditions at 120° C. for 30 min. After cooling to room temperature, the precipitated solid was collected by filtration and dried to afford example 1a5 (62 mg) as a white solid. LC/MS (method 122): RT (UV)=0.67 min, UV-purity 67.4%, ELS-purity 100%, mass observed 246.5.

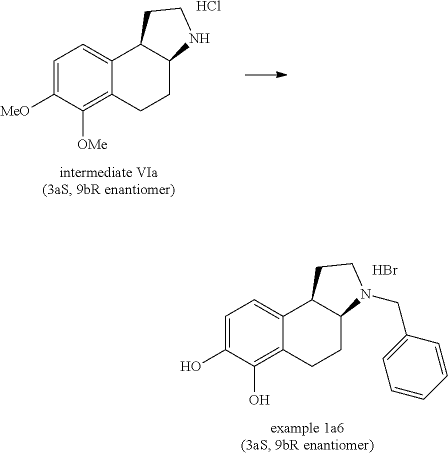

Example 1a6 (3aS,9bR)-3-Benzyl-2,3,3a,4,5,9b-hexahydro-1H-benzo[e]indole-6,7-diol hydrobromide. Intermediate VIa (300 mg) was treated overnight at room temperature with benzyl bromide (198 mg) and potassium carbonate (153 mg) in acetonitrile (10 mL). The volatiles were removed in vacuo, and the residue was purified by chromatography (eluent: heptane/ethyl acetate/triethyl amine 10:10:1) to afford 290 mg of an intermediate. This material was reacted with 48% aqueous HBr (2 mL) under microwave conditions at 120° C. for 30 min. After cooling to room temperature, the precipitated solid was collected by filtration and dried to afford example 1a6 (40 mg) as a white solid. LC/MS (method 122): RT (UV)=0.91 min, UV-purity 86.7%, ELS-purity 100%, mass observed 296.3.

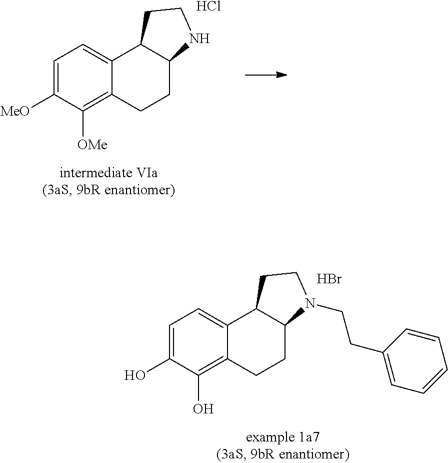

Example 1a7 (3aS,9bR)-3-Phenethyl-2,3,3a,4,5,9b-hexahydro-1H-benzo[e]indole-6,7-diol hydrobromide. Intermediate VIa (300 mg) was treated overnight at room temperature followed by stirring at 70° C. over the weekend with phenethyl bromide (214 mg) and potassium carbonate (153 mg) in acetonitrile (10 mL). The volatiles were removed in vacuo, and the residue was purified by chromatography (eluent: heptane/ethyl acetate/triethyl amine 10:10:1) to afford 310 mg of an intermediate. This material was reacted with 48% aqueous HBr (2 mL) under microwave conditions at 120° C. for 30 min. After cooling to room temperature, the precipitated solid was collected by filtration and dried to afford example 1a7 (70 mg) as a white solid. LC/MS (method 122): RT (UV)=1.01 min, UV-purity 89.0%, ELS-purity 95%, mass observed 310.6.

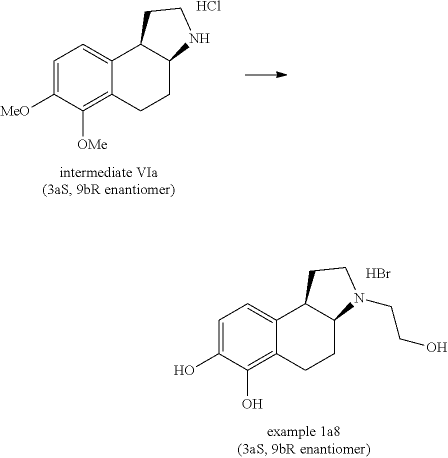

Example 1a8 (3aS,9bR)-3-(2-Hydroxy-ethyl)-2,3,3a,4,5,9b-hexahydro-1H-benzo[e]indole-6,7-diol hydrobromide. Intermediate VIa (300 mg) was treated overnight at room temperature followed by an additional 24 hours at 70° C. and then for four hours at 100° c. with 1-chloro-2-ethoxy ethane (110 mg) and potassium carbonate (153 mg) in acetonitrile (10 mL). The volatiles were removed in vacuo, and the residue was purified by chromatography (eluent: heptane/ethyl acetate/triethyl amine 10:10:1) to afford 160 mg of an intermediate. This material was reacted with 48% aqueous HBr (2 mL) under microwave conditions at 120° C. for 30 min. After cooling to room temperature, the precipitated solid was collected by filtration and dried to afford example 1a1 (140 mg) as a white solid. LC/MS (method 122): RT (UV)=0.53 min, UV-purity 79.7%, ELS-purity 60%, mass observed 250.3.

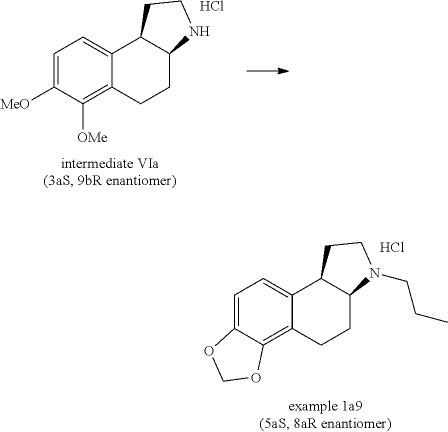

Example 1a9 (5aS,8aR)-6-Propyl-5,5a,6,7,8,8a-hexahydro-4H-1,3-dioxa-6-aza-dicyclopenta[a,f]naphthalene hydrochloride. Intermediate VIa (200 mg) was treated overnight at 60° C. with n-propyl bromide (81 mg) and potassium carbonate (260 mg) in acetonitrile (5 mL). The crude mixture was filtered, and the filtrate was concentrated in vacuo. The residue was treated with 2 mL 48% aqueous HBr (2 mL) under microwave conditions at 120° C. for 20 minutes. After cooling to room temperature, mixture was diluted with acetone at 0° C., and the precipitated material was collected by filtration and dried to afford 100 mg of a grey solid. This material was dissolved in acetonitrile (5 mL) and treated with cesium carbonate (220 mg) and chlorobromoethane (22 microL) under microwave conditions at 150° C. for 20 minutes. The solid was decanted off, and the liquid was concentrated in vacuo. The residue was treated with 2M HCl in diethyl ether (2 mL) and concentrated in vacuo to afford a sticky solid. This material was dissolved in methylene chloride, and concentrated in vacuo to afford example 1a9 as a pale brown foam (52 mg). LC/MS (method 25): RT (UV)=0.99 min, UV-purity 93.3%, ELS-purity 97.7%, mass observed 260.0.

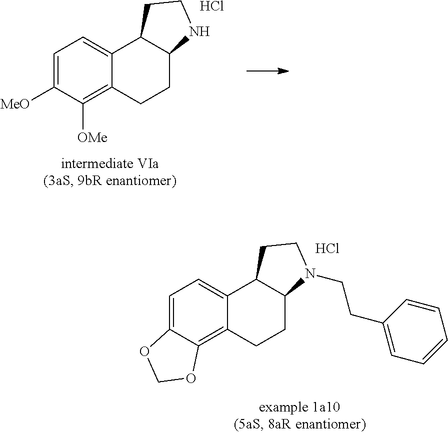

Example 1a10 (5aS,8aR)-6-Phenethyl-5,5a,6,7,8,8a-hexahydro-4H-1,3-dioxa-6-aza-dicyclopenta[a,f]naphthalene hydrochloride. Intermediate VIa (200 mg) was treated overnight at 70° C. with phenethyl bromide (110 microL) and potassium carbonate (260 mg) in acetonitrile (5 mL). The crude mixture was filtered, and the filtrate treated with 48% aqueous HBr to precipitate an intermediate. This material was treated with 48% aqueous HBr (2 mL) under microwave conditions at 120° C. for 20 minutes. After cooling to room temperature, mixture was diluted with acetone at 0° C., and the precipitated solid was collected by filtration and dried to afford 150 mg of a grey solid. This material was dissolved in acetonitrile (5 mL) and treated with cesium carbonate (310 mg) and chlorobromoethane (30 microL) under microwave conditions at 150° C. for 20 minutes. The crude mixture was filtered, and the solid was dissolved in methylene chloride and treated with 2M HCl in diethyl ether and concentrated in vacuo to afford a brown solid. This material was washed with diethyl ether to afford example 1a10 as a pale brown solid (76 mg). LC/MS (method 25): RT (UV)=1.25 min, UV-purity 85.1%, ELS-purity 97.5%, mass observed 310.0.

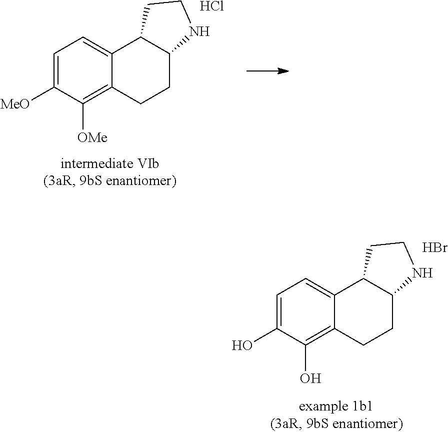

Example 1b1. (3aR,9bS)-2,3,3a,4,5,9b-Hexahydro-1H-benzo[e]indole-6,7-diol hydrobromide. Intermediate VIb (1 mmol) was treated with 48% aqueous HBr (2 mL) under microwave conditions at 120° C. for 30 min. After cooling to room temperature, the precipitated solid was collected by filtration and dried to afford example 1b1 (55 mg) as a white solid. LC/MS (method 122): RT (UV)=0.52 min, UV-purity 83.3%, ELS-purity 100%, mass observed 206.2.

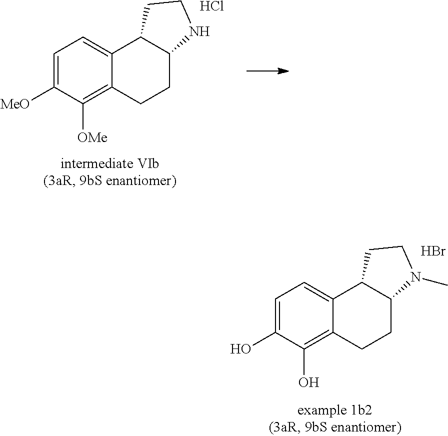

Example 1b2. (3aR,9bS)-3-Methyl-2,3,3a,4,5,9b-hexahydro-1H-benzo[e]indole-6,7-diol hydrobromide. Intermediate VIb (250 mg) was treated overnight at room temperature with formaldehyde (13.4M in water, 100 microL) and sodium cyanoborohydride (116 mg) in methanol (10 mL). The volatiles were removed in vacuo, and the residue was purified by chromatography (eluent: heptane/ethyl acetate/triethyl amine 10:10:1) to afford 200 mg of an intermediate. This material was reacted with 48% aqueous HBr (2 mL) under microwave conditions at 120° C. for 30 min. After cooling to room temperature, the precipitated solid was collected by filtration and dried to afford example 1b2 (76 mg) as a white solid. LC/MS (method 122): RT (UV)=0.54 min, UV-purity 84.4%, ELS-purity 90.6%, mass observed

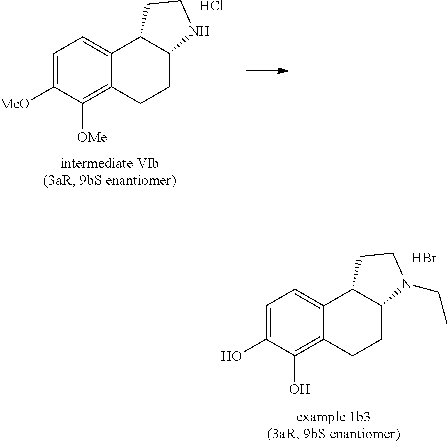

Example 1b3. (3aR,9bS)-3-Ethyl-2,3,3a,4,5,9b-hexahydro-1H-benzo[e]indole-6,7-diol hydrobromide. Intermediate VIb (300 mg) was treated overnight at room temperature with ethyl iodide (181 mg) and potassium carbonate (153 mg) in acetonitrile (10 mL). The volatiles were removed in vacuo, and the residue was purified by chromatography (eluent: heptane/ethyl acetate/triethyl amine 10:10:1) to afford 210 mg of an intermediate. This material was reacted with 48% aqueous HBr (2 mL) under microwave conditions at 120° C. for 30 min. After cooling to room temperature, the precipitated solid was collected by filtration and dried to afford example 1b3 (94 mg) as a white solid. LC/MS (method 122): RT (UV)=0.60 min, UV-purity 82.2%, ELS-purity 100%, mass observed 234.2.

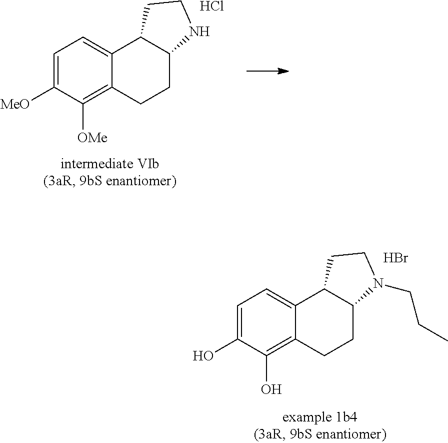

Example 1b4. (3aR,9bS)-3-n-Propyl-2,3,3a,4,5,9b-hexahydro-1H-benzo[e]indole-6,7-diol hydrobromide. Intermediate VIb (300 mg) was treated overnight at room temperature with n-propyl bromide (181 mg) and potassium carbonate (153 mg) in acetonitrile (10 mL). The volatiles were removed in vacuo, and the residue was purified by chromatography (eluent: heptane/ethyl acetate/triethyl amine 10:10:1) to afford 260 mg of an intermediate. This material was reacted with 48% aqueous HBr (2 mL) under microwave conditions at 120° C. for 30 min. After cooling to room temperature, the precipitated solid was collected by filtration and dried to afford example 1b4 (160 mg) as a white solid. LC/MS (method 122): RT (UV)=0.7 min, UV-purity 85.0%, ELS-purity 74.1%, mass observed 248.5.

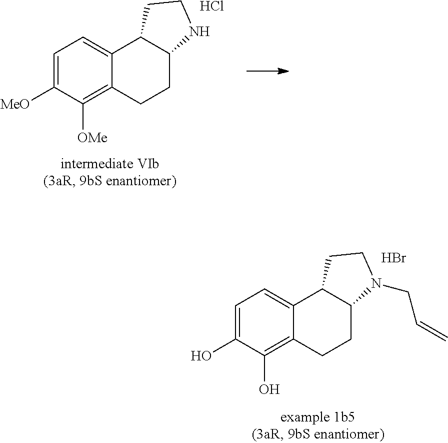

Example 1b5. (3aR,9bS)-3-Allyl-2,3,3a,4,5,9b-hexahydro-1H-benzo[e]indole-6,7-diol hydrobromide. Intermediate VIb (300 mg) was treated overnight at room temperature with allyl bromide (140 mg) and potassium carbonate (153 mg) in acetonitrile (10 mL). The volatiles were removed in vacuo, and the residue was purified by chromatography (eluent: heptane/ethyl acetate/triethyl amine 10:10:1) to afford 200 mg of an intermediate. This material was reacted with 48% aqueous HBr (2 mL) under microwave conditions at 120° C. for 30 min. After cooling to room temperature, the precipitated solid was collected by filtration and dried to afford example 1b5 (90 mg) as a white solid. LC/MS (method 122): RT (UV)=0.67 min, UV-purity 69.7%, ELS-purity 100%, mass observed 246.4.

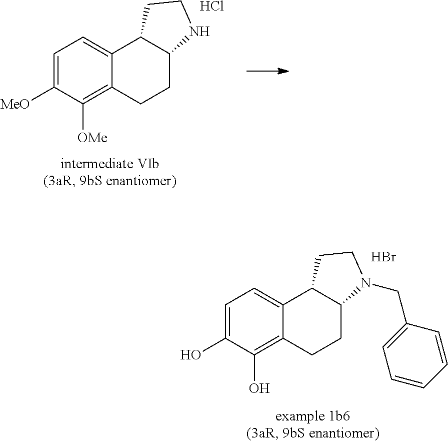

Example 1b6. (3aR,9bS)-3-Benzyl-2,3,3a,4,5,9b-hexahydro-1H-benzo[e]indole-6,7-diol hydrobromide. Intermediate VIb (300 mg) was treated overnight at room temperature with benzyl bromide (198 mg) and potassium carbonate (153 mg) in acetonitrile (10 mL). The volatiles were removed in vacuo, and the residue was purified by chromatography (eluent: heptane/ethyl acetate/triethyl amine 10:10:1) to afford 260 mg of an intermediate. This material was reacted with 48% aqueous HBr (2 mL) under microwave conditions at 120° C. for 30 min. After cooling to room temperature, the precipitated solid was collected by filtration and dried to afford example 1b6 (58 mg) as a white solid. LC/MS (method 122): RT (UV)=0.9 min, UV-purity 85.0%, ELS-purity 95%, mass observed 296.3.

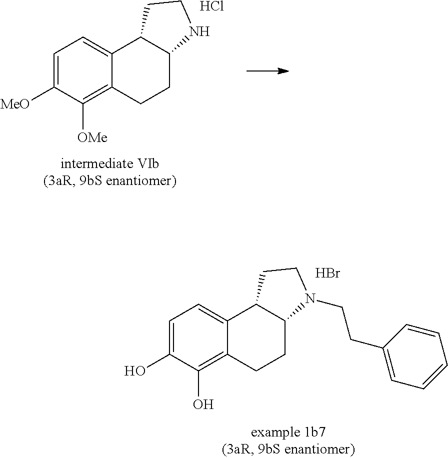

Example 1b7. (3aR,9bS)-3-Phenethyl-2,3,3a,4,5,9b-hexahydro-1H-benzo[e]indole-6,7-diol hydrobromide. Intermediate VIb (300 mg) was treated overnight at room temperature followed by stirring at 70° C. over the weekend with phenethyl bromide (214 mg) and potassium carbonate (153 mg) in acetonitrile (10 mL). The volatiles were removed in vacuo, and the residue was purified by chromatography (eluent: heptane/ethyl acetate/triethyl amine 10:10:1) to afford 315 mg of an intermediate. This material was reacted with 48% aqueous HBr (2 mL) under microwave conditions at 120° C. for 30 min. After cooling to room temperature, the precipitated solid was collected by filtration and dried to afford example 1b7 (110 mg) as a white solid. LC/MS (method 122): RT (UV)=1.01 min, UV-purity 78.1%, ELS-purity 90%, mass observed 310.6.

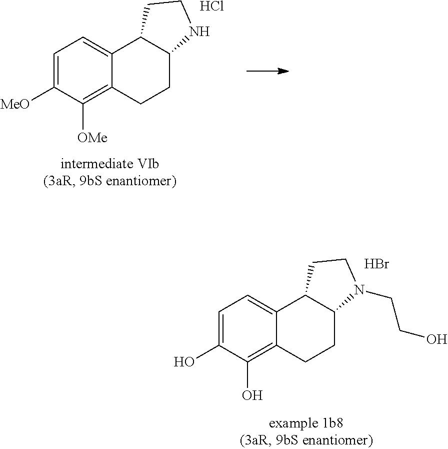

Example 1b8. (3aR,9bS)-3-(2-Hydroxy-ethyl)-2,3,3a,4,5,9b-hexahydro-1H-benzo[e]indole-6,7-diol hydrobromide. Intermediate VIb (300 mg) was treated overnight at room temperature followed by an additional 24 hours at 70° C. and then for four hours at 100° c. with 1-chloro-2-ethoxy ethane (110 mg) and potassium carbonate (153 mg) in acetonitrile (10 mL). The volatiles were removed in vacuo, and the residue was purified by chromatography (eluent: heptane/ethyl acetate/triethyl amine 10:10:1) to afford 66 mg of an intermediate. This material was reacted with 48% aqueous HBr (2 mL) under microwave conditions at 120° C. for 30 min. After cooling to room temperature, the precipitated solid was collected by filtration and dried to afford example 1b8 (50 mg) as a white solid. LC/MS (method 122): RT (UV)=0.52 min, UV-purity 79.7%, ELS-purity 67.6%, mass observed 250.4.

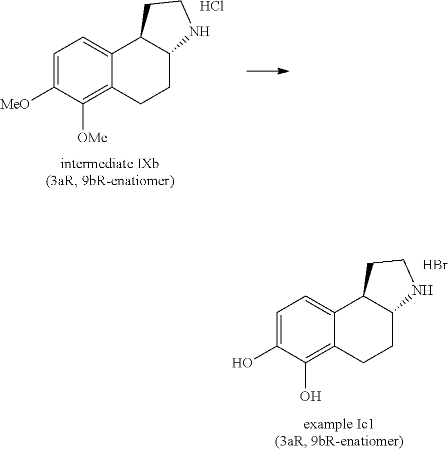

Example 1c1. (3aR,9bR)-2,3,3a,4,5,9b-Hexahydro-1H-benzo[e]indole-6,7-diol hydrobromide. Intermediate IXb (100 mg) was treated with 48% aqueous HBr (2 mL) under microwave conditions at 120° C. for 30 min. The volatiles were removed in vacuo, and the residue was titurated with acetonitrile to give example 1c1 as a white solid (95 mg). LC/MS (method 344): RT (UV)=0.10 min, UV-purity 97.9%, ELS-purity 100%, mass observed 205.9.

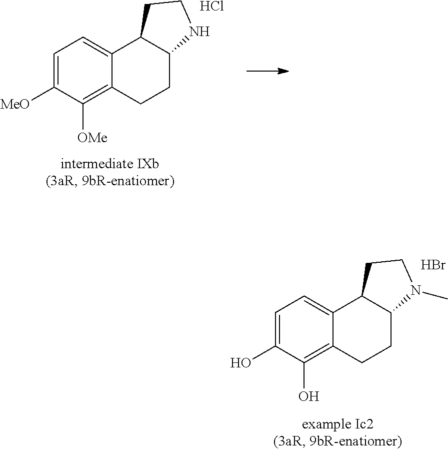

Example 1c2. (3aR,9bR)-3-Methyl-2,3,3a,4,5,9b-hexahydro-1H-benzo[e]indole-6,7-diol hydrobromide. Intermediate IXb (393 mg) was treated with formaline (37% formaldehyde in water; 1 mL) and sodium cyanoborohydride (93 mg) in methanol (5 mL) at room temperature for 1 hour. The volatiles were removed in vacuo, and the residue was dissolved in methylene chloride. The methylene chloride-solution was washed with 5% aqueous sodium carbonate, dried over sodium sulfate, filtered, and concentrated in vacuo. The residue was treated with 48% aqueous HBr (2 mL) under microwave conditions at 120° C. for 30 min. The volatiles were removed in vacuo, and the residue was titurated with acetonitrile to give example 1c2 as a white solid (182 mg). LC/MS (method 344): RT (UV)=0.10 min, UV-purity 100%, ELS-purity 98.7%, mass observed 220.1.

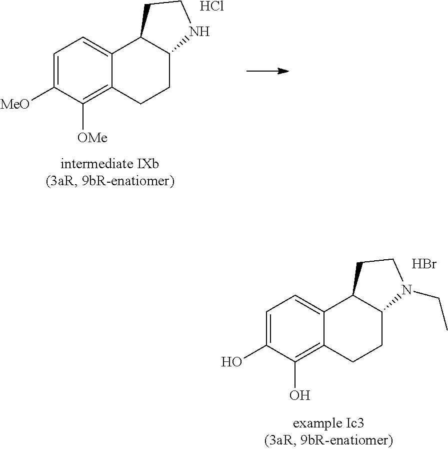

Example 1c3. (3aR,9bR)-3-Ethyl-2,3,3a,4,5,9b-hexahydro-1H-benzo[e]indole-6,7-diol hydrobromide. Intermediate IXb (135 mg) was treated with ethyl iodide (40 microL) and sodium carbonate (126 mg) in acetonitrile (6 mL) at 100° C. for 10 minutes under microwave conditions. The solid was filtered off, and the filtrate was concentrated in vacuo. 100 mg of the residue was treated with 48% aqueous HBr (6 mL) under microwave conditions at 120° C. for 13 min. The volatiles were removed in vacuo, and the residue precipitated from ethanol/diethyl ether to afford example 1c3 as a white solid (110 mg). LC/MS (method 344): RT (UV)=0.11 min, UV-purity 100%, ELS-purity 98.6%, mass observed 233.8.

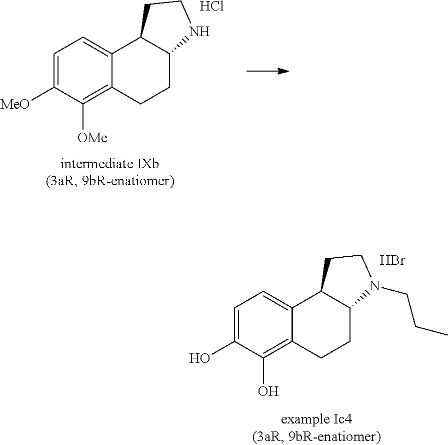

Example 1c4. (3aR,9bR)-3-n-Propyl-2,3,3a,4,5,9b-hexahydro-1H-benzo[e]indole-6,7-diol hydrobromide. Intermediate IXb (270 mg) was treated with n-propyl bromide (100 microL) and sodium carbonate (250 mg) in acetonitrile (6 mL) at 50° C. for 8 hours. The solid was filtered off, and the filtrate was concentrated in vacuo. The residue was treated with 48% aqueous HBr (6 mL) under microwave conditions at 120° C. for 20 minutes. The volatiles were removed in vacuo, and the residue precipitated from ethanol after a treatment with activated charcoal to afford example 1c4 as a white solid (162 mg). LC/MS (method 344): RT (UV)=0.15 min, UV-purity 96%, ELS-purity 99.7%, mass observed 247.9.

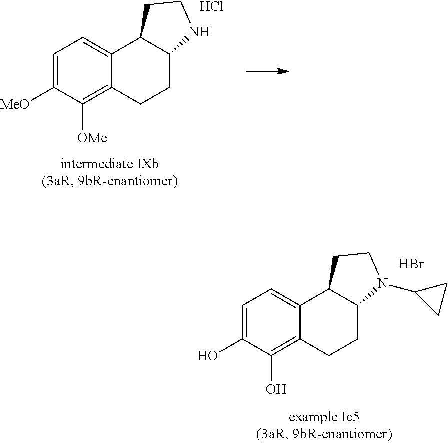

Example 1c5. (3aR,9bR)-3-cyclo-Propyl-2,3,3a,4,5,9b-hexahydro-1H-benzo[e]indole-6,7-diol hydrobromide. Intermediate IXb (270 mg) was treated at 70° C. for 6 hours in a sealed tube containing 4 Å molecular sieves, (1-ethoxy-cyclo-propoxy)-trimethyl-silane (1.20 mL) and sodium cyanoborohydride (280 mg), and acetic acid (0.57 mL). The solids were removed by filtration, and the filtrate was concentrated in vacuo. The residue was partitioned between methylene chloride (50 mL) and 5% aqueous sodium carbonate (10 mL). The organic layer was dried over sodium sulfate, filtered, and concentrated in vacuo. The residue was treated with 48% aqueous HBr (6 mL) under microwave conditions at 120° C. for 13 minutes. The precipitated solid was isolated by filtration and reprecipitated from methanol/diethyl ether after a treatment with activated charcoal to afford example 1c5 as a white solid (178 mg). LC/MS (method 102): RT (UV)=0.59 min, UV-purity 97.2%, ELS-purity 99.7%, mass observed 246.4.

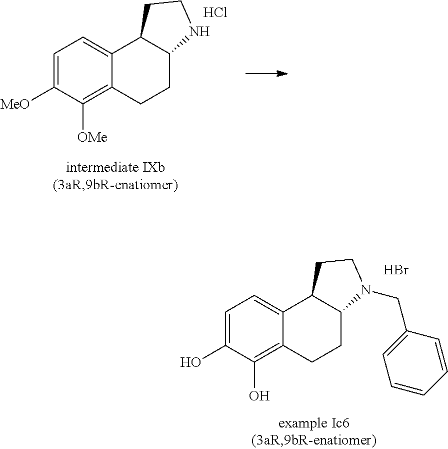

Example 1c6. (3aR,9bR)-3-Benzyl-2,3,3a,4,5,9b-hexahydro-1H-benzo[e]indole-6,7-diol hydrobromide. Intermediate IXb (150 mg) was treated with benzyl bromide (71 microL) and sodium carbonate (126 mg) in acetone (10 mL) at 100° C. for 100 seconds under microwave conditions. The solid was filtered off, and the filtrate was concentrated in vacuo and purified by chromatography (eluent: ethyl acetate). The obtained material was treated with 48% aqueous HBr (6 mL) under microwave conditions at 120° C. for 1000 seconds. The precipitated example 1c6 was obtained as a white solid (17 mg) after filtration. LC/MS (method 102): RT (UV)=0.84 min, UV-purity 98.5%, ELS-purity 99.9%, mass observed 296.3.

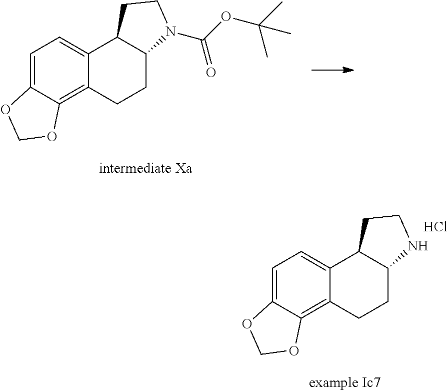

Example 1c7 (5aR,8aR)-5,5a,6,7,8,8a-Hexahydro-4H-1,3-dioxa-6-aza-dicyclopenta[a,f]naphthalene hydrochloride or (5aS,8aS)-5,5a,6,7,8,8a-hexahydro-4H-1,3-dioxa-6-aza-dicyclopenta[a,f]naphthalene hydrochloride (enantiomer of example 1d7). Intermediate Xa (200 mg) was treated with 4.5 M HCl in methanol (3 mL) at room temperature for 1.5 hours. The volatiles were removed in vacuo to afford example 1d7 as a solid (150 mg). LC/MS (method 101): RT (UV)=0.51 min, UV-purity 99%, ELS-purity 99%, mass observed 218.5.

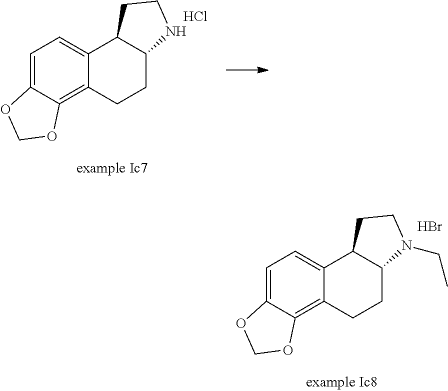

Example 1c8 (5aR,8aR)-6-Ethyl-5,5a,6,7,8,8a-hexahydro-4H-1,3-dioxa-6-aza-dicyclopenta[a,f]naphthalene hydrobromide or (5aS,8aS)-6-ethyl-5,5a,6,7,8,8a-hexahydro-4H-1,3-dioxa-6-aza-dicyclopenta[a,f]naphthalene hydrobromide (enantiomer of example 1d8). Example 1c7 (66 mg) was treated with sodium carbonate (80 mg) and ethyl iodide (40 microL) in acetonitrile at 120° C. for 800 seconds under microwave conditions. The crude mixture was filtered, and the filtrate was concentrated in vacuo. The residue was stirred with acetone to precipitate example 1c8 as a solid (48 mg). LC/MS (method 101): RT (UV)=0.57 min, UV-purity 97%, ELS-purity 100%, mass observed 246.5.

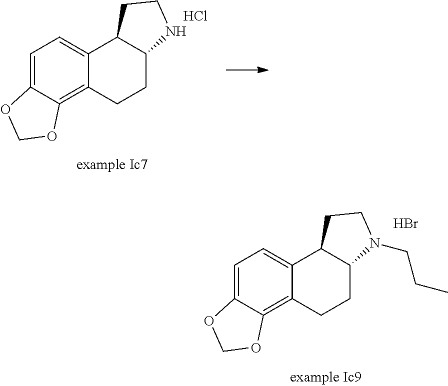

Example 1c9 (5aR,8aR)-6-n-Propyl-5,5a,6,7,8,8a-hexahydro-4H-1,3-dioxa-6-aza-dicyclopenta[a,f]naphthalene hydrobromide or (5aS,8aS)-6-n-propyl-5,5a,6,7,8,8a-hexahydro-4H-1,3-dioxa-6-aza-dicyclopenta[a,f]naphthalene hydrobromide (enantiomer of example 1d9). Example 1c7 (66 mg) was treated with sodium carbonate (80 mg) and n-propyl bromide (42 microL) in acetonitrile at 120° C. for 800 seconds under microwave conditions. The crude mixture was filtered, and the filtrate was concentrated in vacuo. The residue was stirred with acetone to precipitate example 1c9 as a solid (47 mg). LC/MS (method 101): RT (UV)=0.60 min, UV-purity 97%, ELS-purity 98%, mass observed 260.2.

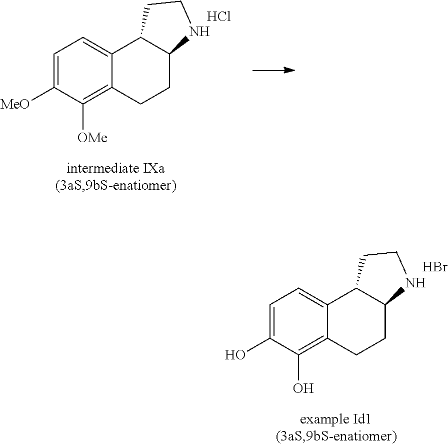

Example 1d1. (3aS,9bS)-2,3,3a,4,5,9b-Hexahydro-1H-benzo[e]indole-6,7-diol hydrobromide. Intermediate IXa (100 mg) was treated with 48% aqueous HBr (2 mL) under microwave conditions at 120° C. for 30 min. The volatiles were removed in vacuo, and the residue was titurated with acetonitrile to give example 1d1 as a white solid (102 mg). LC/MS (method 344): RT (UV)=0.10 min, UV-purity 100%, ELS-purity 98.7%, mass observed

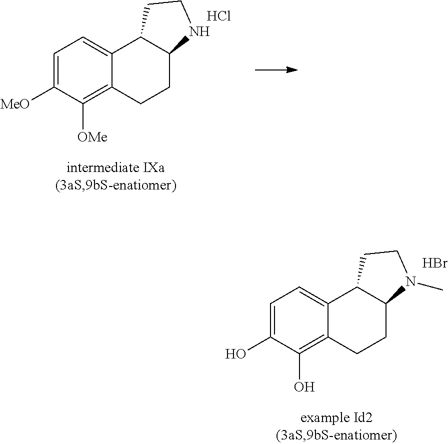

Example 1d2. (3aS,9bS)-3-Methyl-2,3,3a,4,5,9b-hexahydro-1H-benzo[e]indole-6,7-diol hydrobromide. Intermediate IXa (393 mg) was treated with formaline (37% formaldehyde in water; 1 mL) and sodium cyanoborohydride (93 mg) in methanol (5 mL) at room temperature for 1 hour. The volatiles were removed in vacuo, and the residue was dissolved in methylene chloride. The methylene chloride-solution was washed with 5% aqueous sodium carbonate, dried over sodium sulfate, filtered, and concentrated in vacuo. The residue was treated with 48% aqueous HBr (2 mL) under microwave conditions at 120° C. for 30 min. The volatiles were removed in vacuo, and the residue was titurated with acetonitrile to give example 1d2 as a white solid (18 mg). LC/MS (method 344): RT (UV)=0.11 min, UV-purity 95.9%, ELS-purity 98.5%, mass observed 220.2.

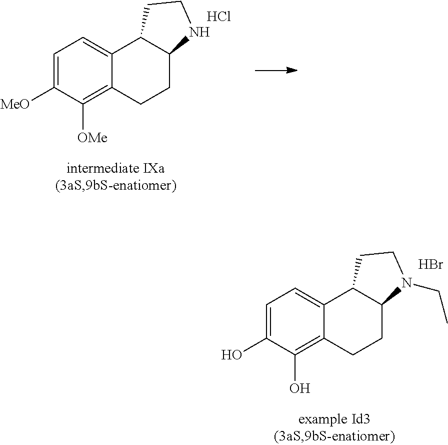

Example 1d3. (3aS,9bS)-3-Ethyl-2,3,3a,4,5,9b-hexahydro-1H-benzo[e]indole-6,7-diol hydrobromide. Intermediate IXa (135 mg) was treated with ethyl iodide (40 microL) and sodium carbonate (126 mg) in acetonitrile (6 mL) at 100° C. for 10 minutes under microwave conditions. The solid was filtered off, and the filtrate was concentrated in vacuo. 100 mg of the residue was treated with 48% aqueous HBr (6 mL) under microwave conditions at 120° C. for 13 min. The volatiles were removed in vacuo, and the residue precipitated from ethanol/diethyl ether to afford example 1d3 as a white solid (56 mg). LC/MS (method 344): RT (UV)=0.11 min, UV-purity 100%, ELS-purity 98.5%, mass observed 233.9.

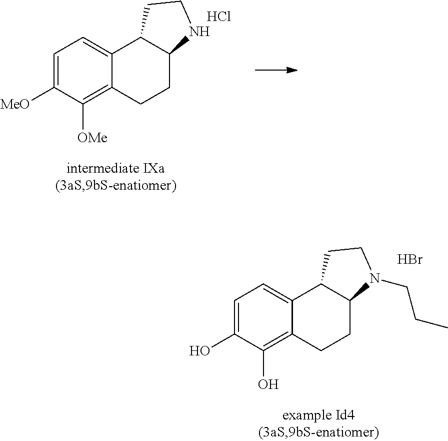

Example 1d4. (3aS,9bS)-3-n-Propyl-2,3,3a,4,5,9b-hexahydro-1H-benzo[e]indole-6,7-diol hydrobromide. Intermediate IXa (270 mg) was treated with n-propyl bromide (100 microL) and sodium carbonate (250 mg) in acetonitrile (6 mL) at 50° C. for 8 hours. The solid was filtered off, and the filtrate was concentrated in vacuo. The residue was treated with 48% aqueous HBr (6 mL) under microwave conditions at 120° C. for 20 minutes. The volatiles were removed in vacuo, and the residue precipitated from ethanol after a treatment with activated charcoal to afford example 1d4 as a white solid (157 mg). LC/MS (method 344): RT (UV)=0.14 min, UV-purity 100%, ELS-purity 99.6%, mass observed 247.9.

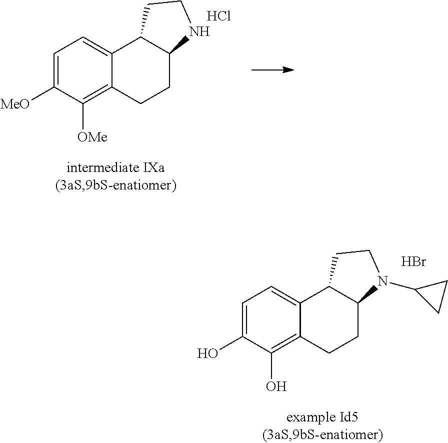

Example 1d5. (3aS,9bS)-3-cyclo-Propyl-2,3,3a,4,5,9b-hexahydro-1H-benzo[e]indole-6,7-diol hydrobromide. Intermediate IXa (270 mg) was treated at 70° C. for 6 hours in a sealed tube containing 4 Å molecular sieves, (1-ethoxy-cyclo-propoxy)-trimethyl-silane (1.20 mL) and sodium cyanoborohydride (280 mg), and acetic acid (0.57 mL). The solids were removed by filtration, and the filtrate was concentrated in vacuo. The residue was partitioned between methylene chloride (50 mL) and 5% aqueous sodium carbonate (10 mL). The organic layer was dried over sodium sulfate, filtered, and concentrated in vacuo. The residue was treated with 48% aqueous HBr (6 mL) under microwave conditions at 120° C. for 13 minutes. The precipitated solid was isolated by filtration and re-precipitated from methanol/diethyl ether after a treatment with activated charcoal to afford example 1d5 as a white solid (195 mg). LC/MS (method 102 ???): RT (UV)=0.59 min, UV-purity 99.1%, ELS-purity 100%, mass observed 246.3.

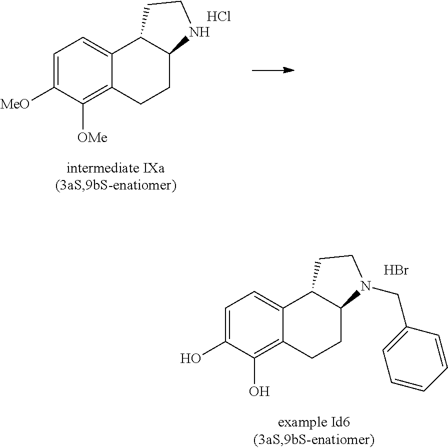

Example 1d6. (3aS,9bS)-3-Benzyl-2,3,3a,4,5,9b-hexahydro-1H-benzo[e]indole-6,7-diol hydrobromide. Intermediate IXa (150 mg) was treated with benzyl bromide (71 microL) and sodium carbonate (126 mg) in acetone (10 mL) at 100° C. for 100 seconds under microwave conditions. The solid was filtered off, and the filtrate was concentrated in vacuo and purified by chromatography (eluent: ethyl acetate). The obtained material was treated with 48% aqueous HBr (6 mL) under microwave conditions at 120° C. for 1000 seconds. The precipitated example 1d6 was obtained as a white solid (18 mg) after filtration. LC/MS (method 102): RT (UV)=0.84 min, UV-purity 100%, ELS-purity 95.1%, mass observed 296.3.

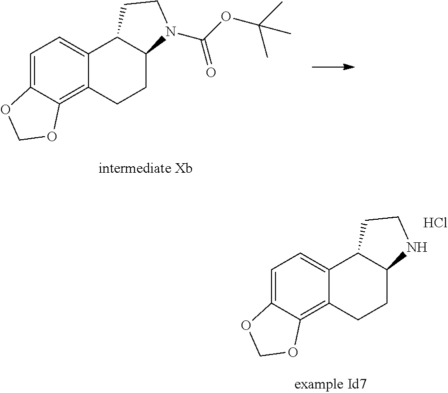

Example 1d7 (5aS,8aS)-5,5a,6,7,8,8a-Hexahydro-4H-1,3-dioxa-6-aza-dicyclopenta[a,f]naphthalene hydrochloride or (5aR,8aR)-5,5a,6,7,8,8a-hexahydro-4H-1,3-dioxa-6-aza-dicyclopenta[a,f]naphthalene hydrochloride (enantiomer of example 1c7). Intermediate Xb (330 mg) was treated with 4.5 M HCl in methanol (5 mL) at room temperature for 1.5 hours. The volatiles were removed in vacuo to afford example 1d7 as a solid (210 mg). LC/MS (method 102): RT (UV)=0.51 min, UV-purity 97%, ELS-purity 100%, mass observed 218.5.

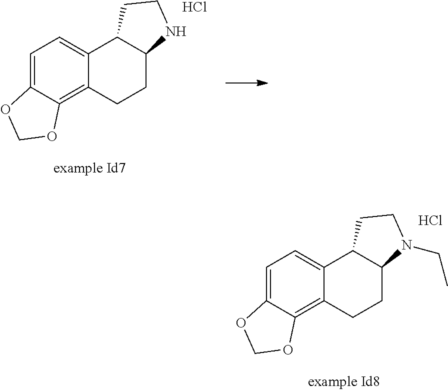

Example 1d8 (5aS,8aS)-6-Ethyl-5,5a,6,7,8,8a-hexahydro-4H-1,3-dioxa-6-aza-dicyclopenta[a,f]naphthalene hydrobromide or (5aR,8aR)-6-ethyl-5,5a,6,7,8,8a-hexahydro-4H-1,3-dioxa-6-aza-dicyclopenta[a,f]naphthalene hydrobromide (enantiomer of example 1c8). Example 1d7 (76 mg) was treated with sodium carbonate (80 mg) and ethyl iodide (40 microL) in acetonitrile at 120° C. for 800 seconds under microwave conditions. The volatiles were removed in vacuo, and the residue was stirred with acetone to precipitate example 1d8 as a solid (52 mg). LC/MS (method 102): RT (UV)=0.58 min, UV-purity 97%, ELS-purity 100%, mass observed 246.5.

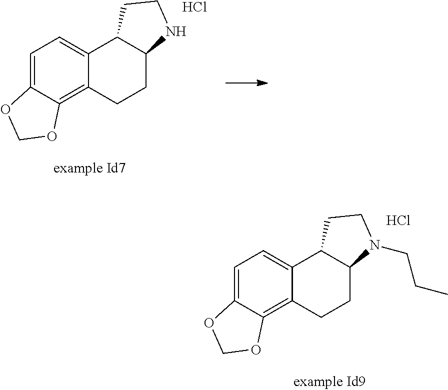

Example 1d9 (5aS,8aS)-6-n-Propyl-5,5a,6,7,8,8a-hexahydro-4H-1,3-dioxa-6-aza-dicyclopenta[a,f]naphthalene hydrobromide or (5aR,8aR)-6-n-propyl-5,5a,6,7,8,8a-hexahydro-4H-1,3-dioxa-6-aza-dicyclopenta[a,f]naphthalene hydrobromide (enantiomer of example 1c9). Example 1d7 (76 mg) was treated with sodium carbonate (80 mg) and n-propyl bromide (42 microL) in acetonitrile at 120° C. for 800 seconds under microwave conditions. The volatiles were removed in vacuo, and the residue was stirred with acetone to precipitate example 1d9 as a solid (80 mg). LC/MS (method 102): RT (UV)=0.60 min, UV-purity 96%, ELS-purity 99%, mass observed 260.2.

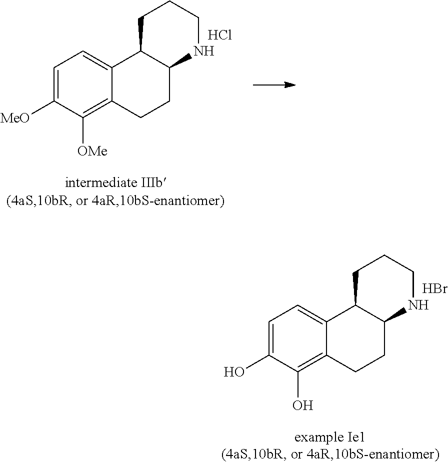

Example 1e1 (4aS,10bR)-1,2,3,4,4a,5,6,10b-Octahydro-benzo[f]quinoline-7,8-diol hydrobromide or (4aR,10bS)-1,2,3,4,4a,5,6,10b-octahydro-benzo[f]quinoline-7,8-diol hydrobromide. Intermediate IIIb′ (0.15 g) was dissolved in 48% aqueous HBr (6 mL). The mixture was heated to 150° C. for 0.5 hours under microwave conditions. The crude mixture was cooled to room temperature and diluted with a little acetone. The resulting mixture was stirred at 0° C. to precipitate a solid. This material was dissolved in hot ethanol and left at 5° C. to precipitate example Ie1 (28.4 mg). LC/MS (method 25): RT (UV)=0.64 min, UV-purity 94.5%, ELS-purity 97.6%, mass observed 220.3.

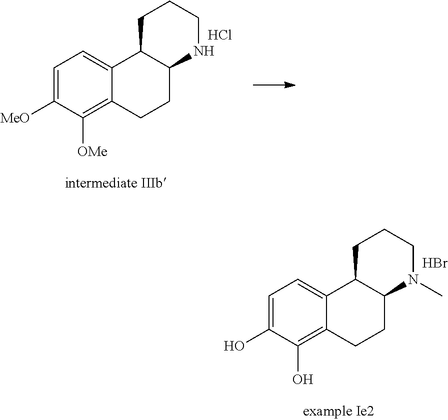

Example let (4aS,10bR)-4-Methyl-1,2,3,4,4a,5,6,10b-octahydro-benzo[f]quinoline-7,8-diol hydrobromide or (4aR,10bS)-4-methyl-1,2,3,4,4a,5,6,10b-octahydro-benzo[f]quinoline-7,8-diol hydrobromide. Intermediate IIIb′ (0.16 g) was dissolved in ethanol and treated with formaldehyde (13.8M in water, 0.04 mL) and sodium cyanoborohydride (0.17 g) at room temperature overnight. The crude mixture was diluted with water (5 mL) and saturated aqueous sodium carbonate (5 mL) and extracted with ethyl acetate (2×25 mL). The combined organic extracts were washed with saturated aqueous sodium chloride (25 mL), dried over magnesium sulfate, filtered, and concentrated in vacuo to afford an intermediate. This material was dissolved in 48% aqueous HBr (3 mL). The mixture was heated to 150° C. for 2×0.5 hours under microwave conditions. The precipitated material was isolated and suspended in hot ethanol. After cooling, the precipitated example 1e2 (27.8 mg) was isolated as an off-white solid. LC/MS (method 25): RT (UV)=0.64 min, UV-purity 91.7%, ELS-purity 98.7%, mass observed 234.2.

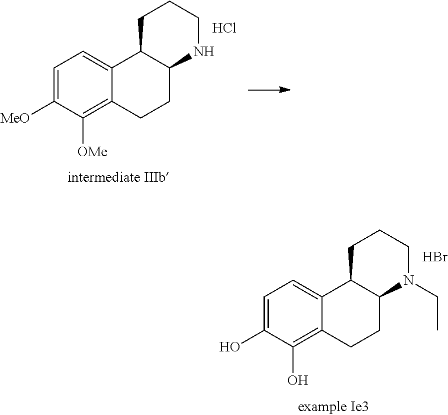

Example 1e3 (4aS,10bR)-4-Ethyl-1,2,3,4,4a,5,6,10b-octahydro-benzo[f]quinoline-7,8-diol hydrobromide or (4aR,10bS)-4-ethyl-1,2,3,4,4a,5,6,10b-octahydro-benzo[f]quinoline-7,8-diol hydrobromide. Intermediate IIIb′ (0.16 g) was dissolved in ethanol and treated with acetaldehyde (0.16 mL) and sodium cyanoborohydride (0.17 g) at room temperature overnight. The crude mixture was diluted with water (5 mL) and saturated aqueous sodium carbonate (5 mL) and extracted with ethyl acetate (2×25 mL). The combined organic extracts were washed with saturated aqueous sodium chloride (25 mL), dried over magnesium sulfate, filtered, and concentrated in vacuo to afford an intermediate. This material was dissolved in 48% aqueous HBr (3 mL). The mixture was heated to 160° C. for 2×0.5 hours under microwave conditions. The volatiles were removed in vacuo. The residual solid was suspended in hot ethanol. After cooling, the precipitated solid was isolated. This material was dissolved in a mixture of methanol (25 mL) and 48% aqueous HBr (0.5 mL) and concentrated in vacuo (repeated twice). The resulting solid was stirred with ethyl acetate and a little methanol, and the precipitated material was discarded. The remaining mixture was concentrated in vacuo, and the residue was stirred with ethyl acetate and a little diethyl ether to precipitate example 1e3 (27.1 mg) as a solid. LC/MS (method 25): RT (UV)=0.68 min, UV-purity 98.4%, ELS-purity 98.6%, mass observed 248.2.

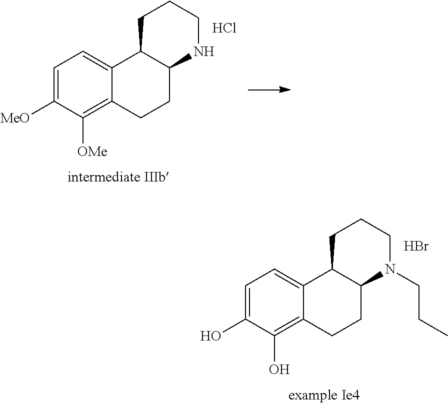

Example 1e4 (4aS,10bR)-4-Propyl-1,2,3,4,4a,5,6,10b-octahydro-benzo[f]quinoline-7,8-diol hydrobromide or (4aR,10bS)-4-propyl-1,2,3,4,4a,5,6,10b-octahydro-benzo[f]quinoline-7,8-diol hydrobromide. Intermediate IIIb′ (0.16 g) was dissolved in ethanol (5 mL) and treated with propanal (0.21 mL) and sodium cyanoborohydride (0.17 g) at room temperature overnight. The crude mixture was diluted with water (5 mL) and saturated aqueous sodium carbonate (5 mL) and extracted with ethyl acetate (2×25 mL). The combined organic extracts were washed with saturated aqueous sodium chloride (25 mL), dried over magnesium sulfate, filtered, and concentrated in vacuo to afford an intermediate. This material was dissolved in 48% aqueous HBr (3 mL). The mixture was heated to 160° C. for 2×0.5 hours under microwave conditions. The volatiles were removed in vacuo. The residual solid was suspended in hot ethanol. After cooling, the precipitated solid was isolated. This material was dissolved in a mixture of ethyl acetate and 48% aqueous HBr and concentrated in vacuo (repeated twice). The resulting solid was stirred with ethanol, and the precipitated example 1e4 (54.9 mg) was isolated. LC/MS (method 25): RT (UV)=0.76 min, UV-purity 96.6%, ELS-purity 98.8%, mass observed 262.3.

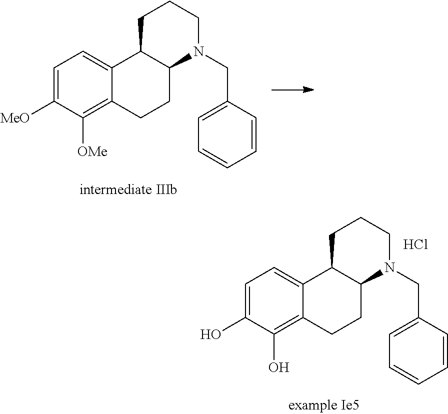

Example 1e5 (4aS,10bR)-4-Benzyl-1,2,3,4,4a,5,6,10b-octahydro-benzo[f]quinoline-7,8-diol hydrobromide or (4aR,10bS)-4-benzyl-1,2,3,4,4a,5,6,10b-octahydro-benzo[f]quinoline-7,8-diol hydrobromide. Intermediate IIIb (100 mg) was suspended in 48% aqueous HBr (2 mL). The mixture was heated to 150° C. for 0.5 hours under microwave conditions. The volatiles were removed in vacuo, and the residue was stirred with acetone. The precipitated solid was isolated and re-precipitated from ethanol to give example 1e5 (54.3 mg) as a solid. LC/MS (method 25): RT (UV)=0.90 min, UV-purity 96.7%, ELS-purity 99.3%, mass observed 310.4.

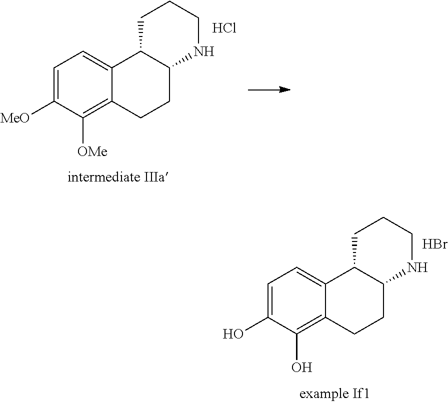

Example 1f1 (4aR,10bS)-1,2,3,4,4a,5,6,10b-Octahydro-benzo[f]quinoline-7,8-diol hydrobromide or (4aS,10bR)-1,2,3,4,4a,5,6,10b-octahydro-benzo[f]quinoline-7,8-diol hydrobromide. Intermediate IIIa′ (0.1 g) was dissolved in 48% aqueous HBr (2 mL). The mixture was heated to 150° C. for 2×0.5 hours under microwave conditions. The crude mixture was cooled to 5° C. overnight, and the precipitated example If1 (58.1 mg) was obtained after washing the solid with water and ethyl acetate. LC/MS (method 25): RT (UV)=0.62 min, UV-purity 95.6%, ELS-purity 99.5%, mass observed 220.2.

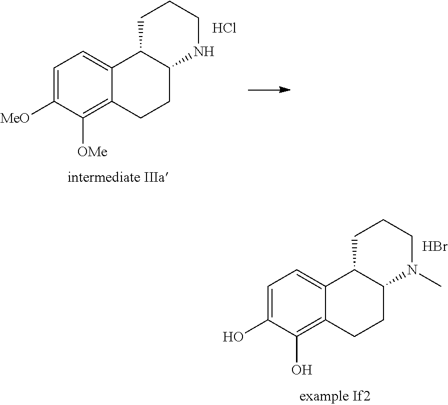

Example 1f2 (4aR,10bS)-4-Methyl-1,2,3,4,4a,5,6,10b-octahydro-benzo[f]quinoline-7,8-diol hydrobromide or (4aS,10bR)-4-methyl-1,2,3,4,4a,5,6,10b-octahydro-benzo[f]quinoline-7,8-diol hydrobromide. Intermediate IIIa′ (0.1 g) was dissolved in ethanol and treated with formaldehyde (13.8M in water, 0.024 mL) and sodium cyanoborohydride (0.11 g) at room temperature overnight. The crude mixture was diluted with water (5 mL) and saturated aqueous sodium carbonate (5 mL) and extracted with ethyl acetate (2×25 mL). The combined organic extracts were washed with saturated aqueous sodium chloride (25 mL), dried over magnesium sulfate, filtered, and concentrated in vacuo to afford an intermediate. This material was dissolved in 48% aqueous HBr (1.5 mL). The mixture was heated to 150° C. for 1 hour under microwave conditions. The volatiles were removed in vacuo. The residue was stirred in methanol and concentrated in vacuo (repeated once). The precipitated material was isolated and stirred in a mixture of ethyl acetate and diethyl ether. After precipitated example 1f2 (49.4 mg) was isolated as an off-white solid. LC/MS (method 25): RT (UV)=0.63 min, UV-purity 94.0%, ELS-purity 99.5%, mass observed 234.2.

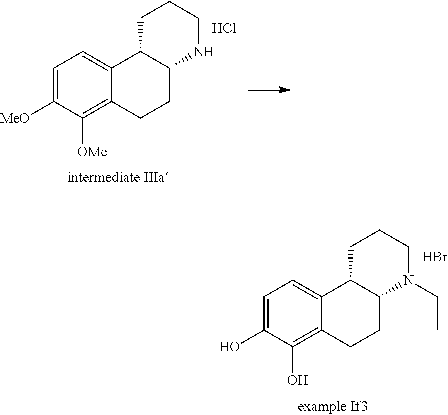

Example 1f3 (4aR,10bS)-4-Ethyl-1,2,3,4,4a,5,6,10b-octahydro-benzo[f]quinoline-7,8-diol hydrobromide or (4aS,10bR)-4-ethyl-1,2,3,4,4a,5,6,10b-octahydro-benzo[f]quinoline-7,8-diol hydrobromide. Intermediate IIIa′ (0.1 g) was dissolved in ethanol and treated with acetaldehyde (0.1 mL) and sodium cyanoborohydride (0.11 g) at room temperature overnight. The crude mixture was diluted with water (5 mL) and saturated aqueous sodium carbonate (5 mL) and extracted with ethyl acetate (2×25 mL). The combined organic extracts were washed with saturated aqueous sodium chloride (25 mL), dried over magnesium sulfate, filtered, and concentrated in vacuo to afford an intermediate, which was purified by chromatography (eluent: ethyl acetate/methanol 9:1→ethyl acetate/methanol/triethyl amine 9:1:1). This resulting intermediate was dissolved in 48% aqueous HBr (1.5 mL). The mixture was heated to 160° C. for 1 hour under microwave conditions. The volatiles were removed in vacuo. The residual solid was suspended in methanol and concentrated in vacuo (repeated twice). The resulting solid was stirred with ethanol and diethyl ether to precipitate example 1f3 (60 mg) as a solid. LC/MS (method 25): RT (UV)=0.68 min, UV-purity 97.6%, ELS-purity 98.6%, mass observed 248.2.

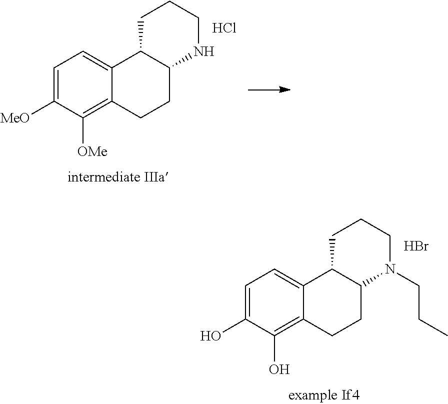

Example 1f4 (4aR,10bS)-4-Propyl-1,2,3,4,4a,5,6,10b-octahydro-benzo[f]quinoline-7,8-diol hydrobromide or (4aS,10bR)-4-propyl-1,2,3,4,4a,5,6,10b-octahydro-benzo[f]quinoline-7,8-diol hydrobromide. Intermediate IIIa′ (0.1 g) was dissolved in ethanol and treated with propanal (0.15 mL) and sodium cyanoborohydride (0.11 g) at room temperature overnight. The crude mixture was diluted with water (5 mL) and saturated aqueous sodium carbonate (5 mL) and extracted with ethyl acetate (2×25 mL). The combined organic extracts were washed with saturated aqueous sodium chloride (25 mL), dried over magnesium sulfate, filtered, and concentrated in vacuo to afford an intermediate, which was purified by chromatography (eluent: ethyl acetate/methanol 9:1→ethyl acetate/methanol/triethyl amine 9:1:1). This resulting intermediate was dissolved in 48% aqueous HBr (1.5 mL). The mixture was heated to 150° C. for 1 hour under microwave conditions. The precipitated example 1f4 (100.6 mg) was isolated as a solid. LC/MS (method 25): RT (UV)=0.75 min, UV-purity 94.6%, ELS-purity 98.5%, mass observed 262.1.

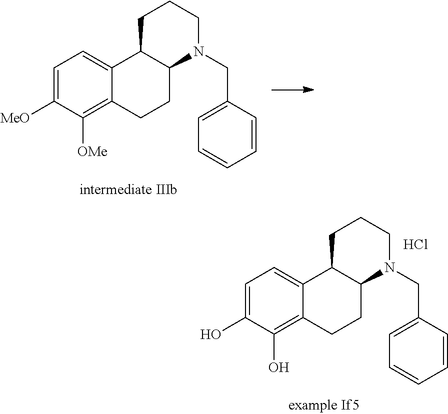

Example 1f5 (4aR,10bS)-4-Benzyl-1,2,3,4,4a,5,6,10b-octahydro-benzo[f]quinoline-7,8-diol hydrobromide or (4aS,10bR)-4-benzyl-1,2,3,4,4a,5,6,10b-octahydro-benzo[f]quinoline-7,8-diol hydrobromide. Intermediate IIIa (125 mg) was suspended in 48% aqueous HBr (2 mL). The mixture was heated to 150° C. for 2×0.5 hours under microwave conditions. The supernatant was decanted off, and the residue was washed with a little water and the supernatant was decanted off. The precipitated oil was dissolved in methanol and acetone was added to precipitate a solid. This material was washed with ethyl acetate and acetone to afford example 1f5 (76.8 mg) as a solid. LC/MS (method 25): RT (UV)=0.93 min, UV-purity 95.3%, ELS-purity 99.3%, mass observed 310.4.

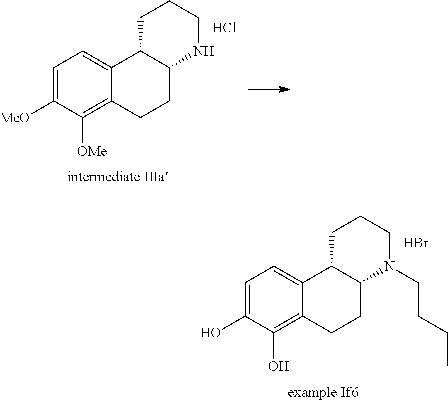

Example 1f6 (4aR,10bS)-4-Butyl-1,2,3,4,4a,5,6,10b-octahydro-benzo[f]quinoline-7,8-diol hydrobromide or (4aS,10bR)-4-butyl-1,2,3,4,4a,5,6,10b-octahydro-benzo[f]quinoline-7,8-diol hydrobromide. Intermediate IIIa′ (0.1 g) was dissolved in ethanol and treated with butanal (0.15 mL) and sodium cyanoborohydride (0.11 g) at room temperature overnight. The crude mixture was diluted with water (5 mL) and saturated aqueous sodium carbonate (5 mL) and extracted with ethyl acetate (2×25 mL). The combined organic extracts were washed with saturated aqueous sodium chloride (25 mL), dried over magnesium sulfate, filtered, and concentrated in vacuo to afford an intermediate, which was purified by chromatography (eluent: ethyl acetate/methanol 9:1→ethyl acetate/methanol/triethyl amine 9:1:1). This resulting intermediate was dissolved in 48% aqueous HBr (1.5 mL). The mixture was heated to 150° C. for 1 hour under microwave conditions. The volatiles were removed in vacuo. The residual solid was suspended in methanol and concentrated in vacuo (repeated twice). The resulting solid was stirred with ethanol and diethyl ether to precipitate example 1f6 (21.8 mg) as a solid. LC/MS (method 25): RT (UV)=0.85 min, UV-purity 90.2%, ELS-purity 99.6%, mass observed 276.2.

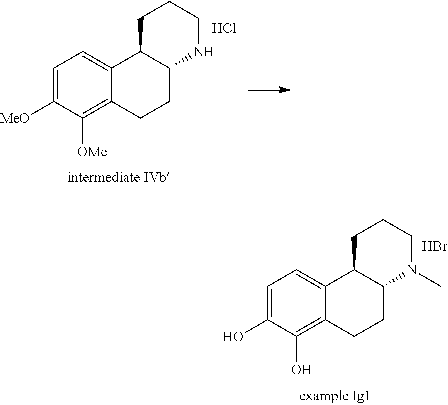

Example 1g1 (4aR,10bR)-4-Methyl-1,2,3,4,4a,5,6,10b-octahydro-benzo[f]quinoline-7,8-diol hydrobromide or (4aS,10bS)-4-methyl-1,2,3,4,4a,5,6,10b-octahydro-benzo[f]quinoline-7,8-diol hydrobromide. Intermediate IVb′ (0.2 g) was dissolved in ethanol (5 mL) and treated with formaldehyde (13.8M in water, 0.055 mL) and sodium cyanoborohydride (0.25 g) overnight at room temperature. The crude mixture was concentrated in vacuo, and the residue was partitioned between saturated aqueous sodium carbonate (5 mL), water (5 mL), and ethyl acetate (25 mL). The aqueous layer was extracted with ethyl acetate (25 mL). The combined organic layers were washed with saturated aqueous sodium chloride (25 mL), dried over magnesium sulfate, filtered, and the concentrated in vacuo. The residue was purified by chromatography (eluent: ethyl acetate/methanol 9:1→ethyl acetate/4M NH₃ in methanol 9:1) to afford an intermediate. This material was treated with 48% aqueous HBr (3 mL) at 150° C. for 0.5 hours under microwave conditions. The crude mixture was cooled to 0° C. to precipitate a solid. This material was suspended in warm EtOH and then cooled to room temperature to afford example 1g1 (80.7 mg) as a solid. LC/MS (method 25): RT (UV)=0.64 min, UV-purity 100%, ELS-purity 98.8%, mass observed 234.1.

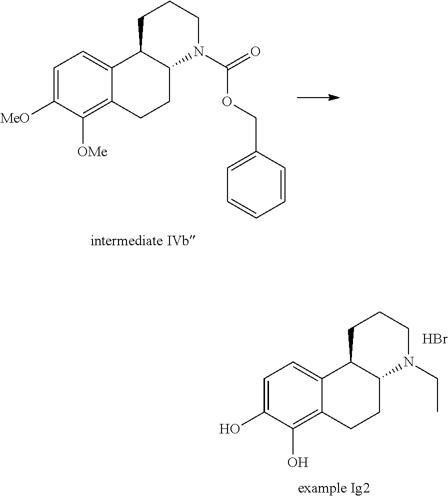

Example 1g2 (4aR,10bR)-4-Ethyl-1,2,3,4,4a,5,6,10b-octahydro-benzo[f]quinoline-7,8-diol hydrobromide or (4aS,10bS)-4-ethyl-1,2,3,4,4a,5,6,10b-octahydro-benzo[f]quinoline-7,8-diol hydrobromide. Intermediate IVb″ (85 mg) was dissolved in ethanol (20 mL). Acetic acid (3 drops), acetaldehyde (0.06 mL), and 10% Pd/C (35 mg) were added, and the mixture was treated with hydrogen gas (3 bar) for 2.5 hours. The catalyst was filtered off, and the filtrate was concentrated in vacuo. The residue was purified by chromatography (eluent: ethyl acetate/methanol/triethyl amine 9:1:1). The resulting intermediate was dissolved in 48% aqueous HBr (2 mL) at 150° C. for 0.5 hours under microwave conditions. The crude mixture was cooled to 5° C. to precipitate example 1g2 (18.4 mg) as a solid. LC/MS (method 25): RT (UV)=0.64 min, UV-purity 94.0%, ELS-purity 98.5%, mass observed 248.1.

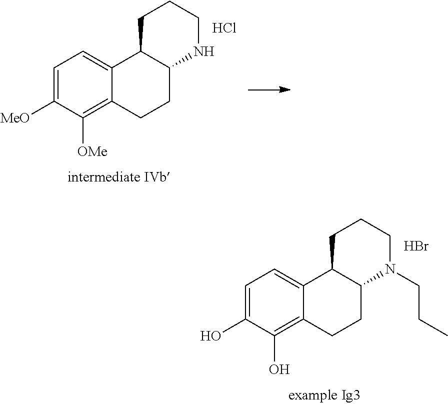

Example 1g3 (4aR,10bR)-4-Propyl-1,2,3,4,4a,5,6,10b-octahydro-benzo[f]quinoline-7,8-diol hydrobromide or (4aS,10bS)-4-propyl-1,2,3,4,4a,5,6,10b-octahydro-benzo[f]quinoline-7,8-diol hydrobromide. Intermediate IVb′ (2.8 g) was suspended in ethanol (50 mL) and treated with propanal (3.6 mL) and sodium cyanoborohydride (3.05 g) overnight at room temperature. The crude mixture diluted with saturated aqueous sodium carbonate (15 mL) and water (15+50 mL), and the intermediate was extracted into ethyl acetate (50 mL). The aqueous layer was extracted with ethyl acetate (50 mL). The combined organic layers were washed with saturated aqueous sodium chloride, dried over magnesium sulfate, filtered, and concentrated in vacuo. The residue was treated with 37% aqueous HCl (30 mL) in ethyl acetate (50 mL) at room temperature overnight. The volatiles were removed in in vacuo, and the residue was dissolved in a mixture of ethyl acetate (50 mL) and water (50 mL), cooled to 0° C., and the pH was adjusted to ˜12 with sodium hydroxide. The organic layer was washed with saturated aqueous sodium chloride, dried over magnesium sulfate, filtered, and concentrated in vacuo. The residue was purified by chromatography (eluent: ethyl acetate/methanol 9:1) to afford an intermediate. This material was split into two equal portion; each of which was treated with 48% aqueous HBr (12.5 mL) at 150° C. for 1 hour under microwave conditions. The crude mixtures were combined and cooled to 0° C. to precipitate a solid, which was collected by filtration. The filtrate was concentrated in vacuo to an approximate volume of 10 mL, before it was cooled to 0° C. to precipitate a second crop of the solid. The combined solids were re-precipitated from methanol/ethanol to afford example 1g3 (2.11 g) as a solid. LC/MS (method 25): RT (UV)=0.75 min, UV-purity 98.9%, ELS-purity 96.7%, mass observed 262.3.

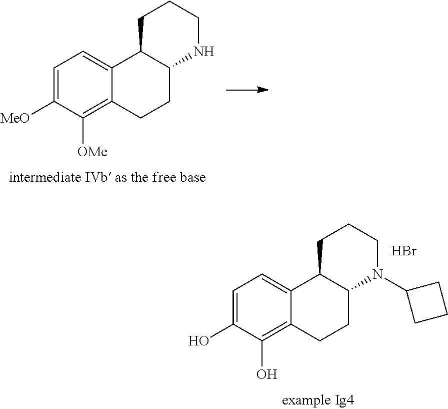

Example 1g4 (4aR,10bR)-4-cyclo-Butyl-1,2,3,4,4a,5,6,10b-octahydro-benzo[f]quinoline-7,8-diol hydrobromide or (4aS,10bS)-4-cyclo-butyl-1,2,3,4,4a,5,6,10b-octahydro-benzo[f]quinoline-7,8-diol hydrobromide. Intermediate IVb′ (250 mg of the free base) was dissolved in 1,2-dichloroethane. Sodium cyanoborohydride (321 mg) and cyclobutanone (0.38 mL) were added, and the mixture was stirred at room temperature overnight. Next morning, a little sodium cyanoborohydride was added, and the mixture was stirred at room temperature over the weekend. The reaction was quenched with water and basified. The aqueous layer was extracted with 1,2-dichloroethane. The combined organic layers were washed with saturated aqueous sodium chloride, dried over magnesium sulfate, filtered, and concentrated in vacuo. The residue was purified by chromatography (eluent: ethyl acetate/methanol/triethyl amine 9:1:1) to give an intermediate. This material was dissolved in 48% aqueous HBr (2 mL) at 150° C. for 0.5 hours under microwave conditions. The crude mixture was cooled to 5° C. to precipitate example 1g4 (148 mg). LC/MS (method 102): RT (UV)=0.71 min, UV-purity 81.8%, ELS-purity 100%, mass observed 274.5.

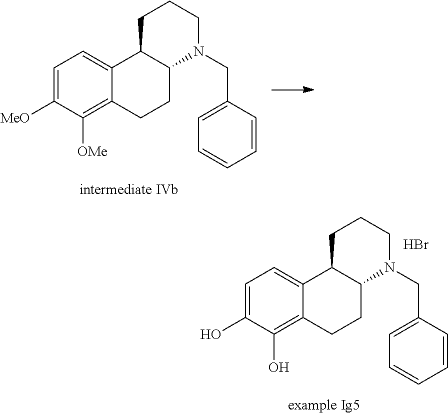

Example 1g5 (4aR,10bR)-4-Benzyl-1,2,3,4,4a,5,6,10b-octahydro-benzo[f]quinoline-7,8-diol hydrobromide or (4aS,10bS)-4-benzyl-1,2,3,4,4a,5,6,10b-octahydro-benzo[f]quinoline-7,8-diol hydrobromide. Intermediate IVb (220 mg) was suspended in 48% aqueous HBr (4.5 mL) at 150° C. for 2×0.5 hour under microwave conditions. The supernatant was decanted off, and the residue was precipitated from methanol/ethanol to afford example 1g5 (18.4 mg) as a solid. LC/MS (method 25): RT (UV)=1.00 min, UV-purity 92.1%, ELS-purity 99.4%, mass observed 310.4.

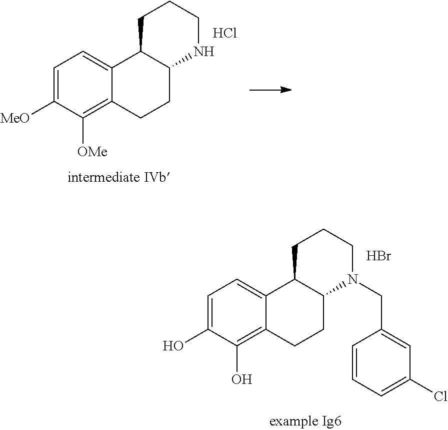

Example 1g6 (4aR,10bR)-4-(3-Chloro-benzyl)-1,2,3,4,4a,5,6,10b-octahydro-benzo[f]quinoline-7,8-diol hydrobromide or (4aS,10bS)-4-(3-chloro-benzyl)-1,2,3,4,4a,5,6,10b-octahydro-benzo[f]quinoline-7,8-diol hydrobromide. Intermediate IVb′ (0.5 g) was partitioned between ethyl acetate and aqueous base. The organic layer was dried over magnesium sulfate, filtered, and concentrated in vacuo. Half of the resulting material was dissolved in DMF (5 mL) and reacted with sodium hydride (60% dispersion; 0.6 g) for 0.5 hours under mild heating. The resulting mixture was treated with 3-chloro-benzyl chloride (0.17 mL) at room temperature for 2 days. The crude mixture was poured into ice/water, and the precipitated material was collected and purified by chromatography (eluent: heptanes/ethyl acetate 2:1) to afford an intermediate. 156 mg of this material was dissolved in methylene chloride (5 mL) and cooled to −78° C. BBr₃ (1M in methylene chloride; 0.84 mL) was added, and the mixture was allowed to warm to room temperature. The mixture was cooled to −60° C. before it was quenched with methanol (5 mL). The mixture was warmed to room temperature, and the precipitated solid was collected. This material was re-precipitated from warm methanol/EtOH. The resulting solid was re-precipitated from methanol to give example 1g6 (61.9 mg). LC/MS (method 25): RT (UV)=1.07 min, UV-purity 93.2%, ELS-purity 98.5%, mass observed 344.2.

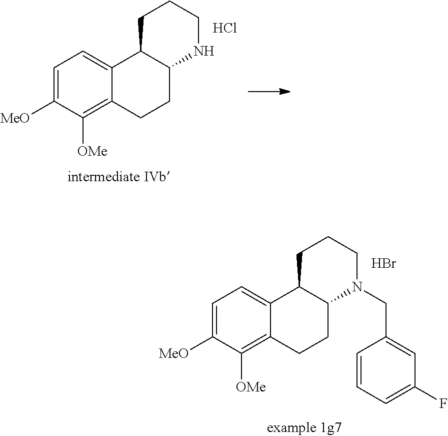

Example 1g7 (4aR,10bR)-4-(3-Fluoro-benzyl)-1,2,3,4,4a,5,6,10b-octahydro-benzo[f]quinoline-7,8-diol hydrobromide or (4aS,10bS)-4-(3-fluoro-benzyl)-1,2,3,4,4a,5,6,10b-octahydro-benzo[f]quinoline-7,8-diol hydrobromide. Intermediate IVb′ (0.5 g) was partitioned between ethyl acetate and aqueous base. The organic layer was dried over magnesium sulfate, filtered, and concentrated in vacuo. Half of the resulting material was dissolved in DMF (5 mL) and reacted with sodium hydride (60% dispersion; 0.6 g) for 0.5 hours under mild heating. The resulting mixture was treated with 3-fluoro-benzyl chloride (0.16 mL) at room temperature for 2 days. The crude mixture was poured into ice/water, and the precipitated material was collected and purified by chromatography (eluent: heptanes/ethyl acetate 2:1) to afford an intermediate. 220 mg of this material was dissolved in m (5 mL) and cooled to −78° C. BBr₃ (1M in methylene chloride; 1.23 mL) was added, and the mixture was allowed to warm to room temperature. The mixture was cooled to −60° C. before it was quenched with methanol (5 mL). The mixture was warmed to room temperature, and the precipitated solid was collected. This material was re-precipitated from warm methanol/EtOH. The resulting solid was re-precipitated from methanol to give example 1g7 (114 mg). LC/MS (method 25): RT (UV)=1.02 min, UV-purity 99.6%, ELS-purity 99.4%, mass observed 328.2.

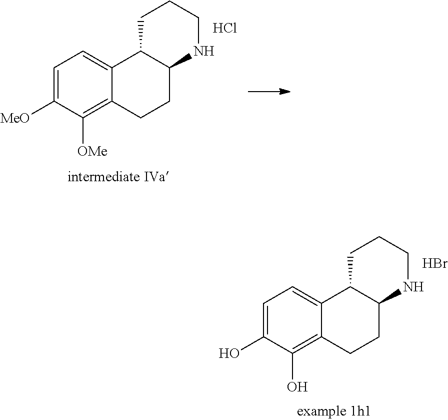

Example 1h1 (4aS,10bS)-1,2,3,4,4a,5,6,10b-Octahydro-benzo[f]quinoline-7,8-diol hydrobromide or (4aR,10bR)-1,2,3,4,4a,5,6,10b-octahydro-benzo[f]quinoline-7,8-diol hydrobromide. Intermediate IVa′ (0.355 g) was free-based by partitioning between ethyl acetate and 4M aqueous sodium hydroxide. The organic layer was washed with saturated aqueous sodium chloride, dried over magnesium sulfate, filtered, and concentrated in vacuo. The residue was dissolved in 48% aqueous HBr (3 mL) at 150° C. for 1 hour under microwave conditions. The crude mixture was cooled to 5° C. to precipitate example 1h1 (0.24 g) as a solid. LC/MS (method 25): RT (UV)=0.56 min, UV-purity 98.5%, ELS-purity 99.6%, mass observed 220.2.

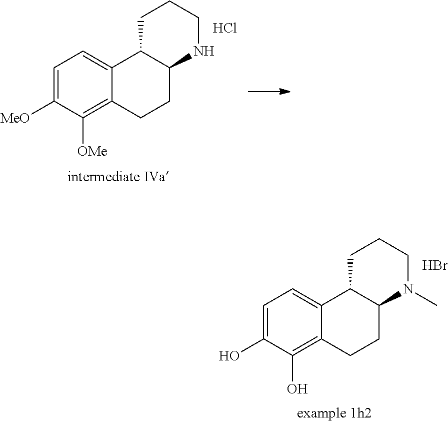

Example 1h2 (4aS,10bS)-4-Methyl-1,2,3,4,4a,5,6,10b-octahydro-benzo[f]quinoline-7,8-diol hydrobromide or (4aR,10bR)-4-methyl-1,2,3,4,4a,5,6,10b-octahydro-benzo[f]quinoline-7,8-diol hydrobromide. Intermediate IVa′ (0.2 g) was stirred with formaldehyde (13.8M in water; 0.048 mL) and sodium cyanoborohydride (0.22 g) in a mixture of ethanol (5 mL) and acetic acid (drops) at room temperature overnight. The crude mixture was concentrated in vacuo. The residue was stirred in a mixture of ethyl acetate and 37% aqueous HCl overnight, before it was concentrated in vacuo. The residue was partitioned between ethyl acetate and aqueous base. The aqueous layer was extracted with ethyl acetate. The combined organic layers were washed with saturated aqueous sodium chloride, dried over magnesium sulfate, filtered, and concentrated in vacuo. The residue was dissolved in 48% aqueous HBr (2 mL) at 150° C. for 0.5 hours under microwave conditions. The crude mixture was cooled to 5° C. to precipitate a solid. This material was suspended in diethyl ether to afford example 1h2 (92.4 mg) as a solid. LC/MS (method 25): RT (UV)=0.62 min, UV-purity 94.5%, ELS-purity 98.7%, mass observed 234.2.

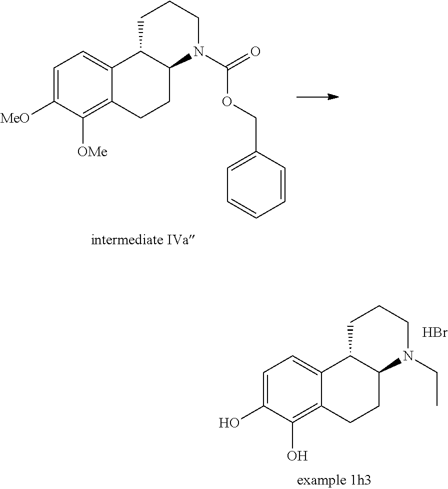

Example 1h3 (4aS,10bS)-4-Ethyl-1,2,3,4,4a,5,6,10b-octahydro-benzo[f]quinoline-7,8-diol hydrobromide or (4aR,10bR)-4-ethyl-1,2,3,4,4a,5,6,10b-octahydro-benzo[f]quinoline-7,8-diol hydrobromide. Intermediate IVa″ (90 mg) was dissolved in ethanol (20 mL). Acetic acid (3 drops), acetaldehyde (0.08 mL), and 10% Pd/C (50 mg) were added, and the mixture was treated with hydrogen gas (3 bar) overnight. The catalyst was filtered off, and the filtrate was concentrated in vacuo. The residue was purified twice by chromatography (eluent: ethyl acetate/methanol/triethyl amine 9:1:1). The resulting intermediate was dissolved in 48% aqueous HBr (1 mL) at 150° C. for 0.5 hours under microwave conditions. The crude mixture was cooled to 5° C. to precipitate example 1h3 (32.6 mg) as a solid. LC/MS (method 14): RT (UV)=0.60 min, UV-purity 86.1%, ELS-purity 99.7%, mass observed 248.1.

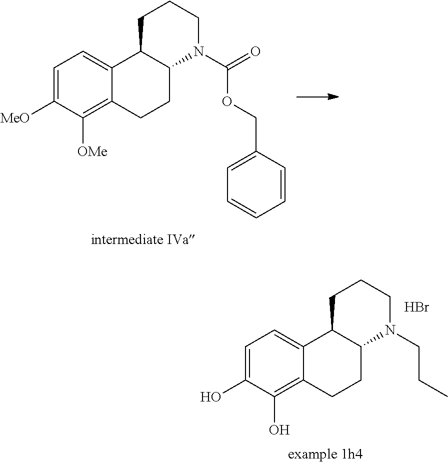

Example 1h4 (4aS,10bS)-4-Propyl-1,2,3,4,4a,5,6,10b-octahydro-benzo[f]quinoline-7,8-diol hydrobromide or (4aR,10bR)-4-propyl-1,2,3,4,4a,5,6,10b-octahydro-benzo[f]quinoline-7,8-diol hydrobromide. Intermediate IVa″ (90 mg) was dissolved in ethanol (20 mL). Acetic acid (3 drops), propanal (0.085 mL), and 10% Pd/C (35 mg) were added, and the mixture was treated with hydrogen gas (3 bar) overnight. The catalyst was filtered off, and the filtrate was concentrated in vacuo. The residue was purified by chromatography (eluent: ethyl acetate/methanol/triethyl amine 9:1:1). The resulting intermediate was dissolved in 48% aqueous HBr (1.5 mL) at 150° C. for 0.5 hours under microwave conditions. The crude mixture was cooled to 5° C. to precipitate a solid. This material was re-precipitated from ethanol to afford example 1h4 (15 mg) as a solid. LC/MS (method 20): RT (UV)=0.66 min, UV-purity 98.8%, ELS-purity 98.2%, mass observed 262.1.

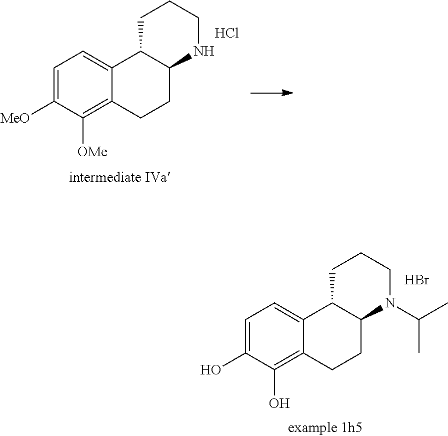

Example 1h5 (4aS,10bS)-4-iso-Propyl-1,2,3,4,4a,5,6,10b-octahydro-benzo[f]quinoline-7,8-diol hydrobromide or (4aR,10bR)-4-iso-propyl-1,2,3,4,4a,5,6,10b-octahydro-benzo[f]quinoline-7,8-diol hydrobromide. Intermediate IVa′ (0.5 g), cesium carbonate (1.15 g), and 2-iodo-propane (2 mL) were stirred in DMF (15 mL) at room temperature for 2 days. The crude mixture was poured in water and extracted with diethyl ether. The organic extract was washed with saturated aqueous sodium chloride, dried over magnesium sulfate, filtered, and concentrated in vacuo. The residue was dissolved in methylene chloride (3 mL) and cooled to −78° C. before BBr₃ (1M in methylene chloride; 2.7 mL) was added. The mixture was allowed to warm to room temperature and stirred for 2 hours. After cooling the −78° C., the reaction was quenched with methanol, warmed to room temperature and partially concentrated in vacuo. Diethyl ether was added to precipitate example 1h5 (470 mg). LC/MS (method 25): RT (UV)=0.75 min, UV-purity 93.6%, ELS-purity 96.6%, mass observed 262.2

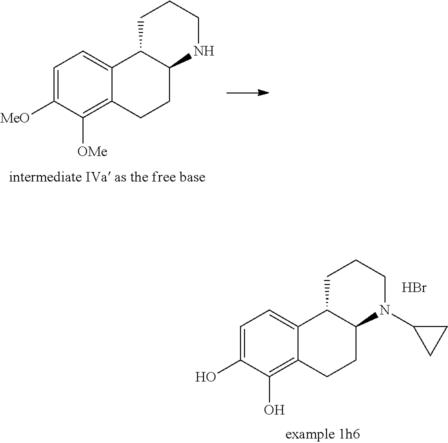

Example 1h6 (4aS,10bS)-4-cyclo-Propyl-1,2,3,4,4a,5,6,10b-octahydro-benzo[f]quinoline-7,8-diol hydrobromide or (4aR,10bR)-4-cyclo-propyl-1,2,3,4,4a,5,6,10b-octahydro-benzo[f]quinoline-7,8-diol hydrobromide. Intermediate IVa′ (250 mg of the free base), (1-ethoxy-cyclo-propoxy)-trimethyl-silane (1.21 mL), sodium cyanoborohydride (317 mg), and 4 Å molecular sieves were suspended in a mixture of methanol (2.5 mL) and acetic acid (0.5 mL). The mixture was stirred at 75° C. overnight in a sealed vial. The crude mixture was filtered, and the filtrate was concentrated in vacuo. The residue was partitioned between ethyl acetate and water (pH adjusted to 6-7). The organic layer was washed with saturated aqueous sodium chloride, dried over magnesium sulfate, filtered, and concentrated in vacuo. The residue was partitioned between ethyl acetate and 0.5% aqueous HCl. The aqueous layer was basified and extracted with ethyl acetate. The organic layer was washed with saturated aqueous sodium chloride, dried over magnesium sulfate, filtered, and concentrated in vacuo. The residue was dissolved in 48% aqueous HBr (1.5 mL) and stirred at 150° C. for 1 hour under microwave conditions. The crude mixture was left at 5° C. overnight. The precipitated solid was suspended in a mixture of methylene chloride and ethanol to afford example 1h6 (158 mg) as a solid. LC/MS (method 102): RT (ELS)=0.63 min, very poor UV-absorbance, ELS-purity 100%, mass observed 260.3.

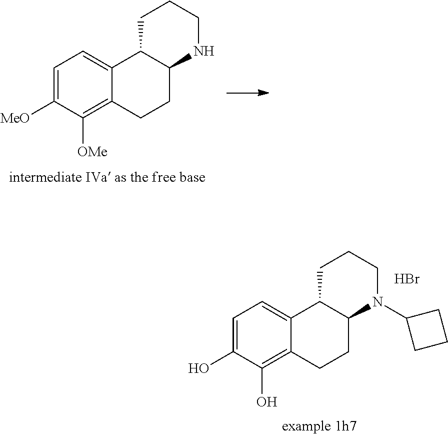

Example 1h7 (4aS,10bS)-4-cyclo-Butyl-1,2,3,4,4a,5,6,10b-octahydro-benzo[f]quinoline-7,8-diol hydrobromide or (4aR,10bR)-4-cyclo-butyl-1,2,3,4,4a,5,6,10b-octahydro-benzo[f]quinoline-7,8-diol hydrobromide. Intermediate IVa′ (250 mg of the free base) was dissolved in 1,2-dichloroethane (5 mL). Sodium cyanoborohydride (321 mg) and cyclobutanone (0.38 mL) were added, and the mixture was stirred at room temperature overnight. The reaction was quenched with water and concentrated in vacuo. The residue was partitioned between methylene chloride and 4M aqueous sodium hydroxide. The organic layer was washed with saturated aqueous sodium chloride, dried over magnesium sulfate, filtered, and concentrated in vacuo. The residue was dissolved in 48% aqueous HBr (2 mL) and stirred at 150° C. for 0.5 hours under microwave conditions. The precipitated material was collected, and re-precipitated from ethanol to afford example 1h7 (92 mg). LC/MS (method 102): RT (UV)=0.69 min, UV-purity 83.1%, ELS-purity 98.2%, mass observed 274.2.

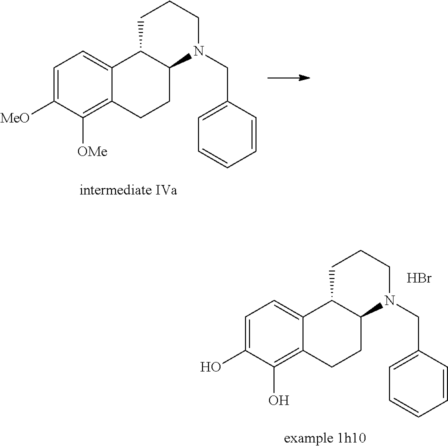

Example 1h10 (4aS,10bS)-4-Benzyl-1,2,3,4,4a,5,6,10b-octahydro-benzo[f]quinoline-7,8-diol hydrobromide or (4aR,10bR)-4-benzyl-1,2,3,4,4a,5,6,10b-octahydro-benzo[f]quinoline-7,8-diol hydrobromide. Intermediate IVa (220 mg) was suspended in 48% aqueous HBr (2.0 mL) at 150° C. for 2×0.5 hours under microwave conditions. The precipitated solid was re-precipitated from methanol/ethanol to afford example 1h10 (72 mg) as a solid. LC/MS (method 25): RT (UV)=0.98 min, UV-purity 96.7%, ELS-purity 99.4%, mass observed 310.4.

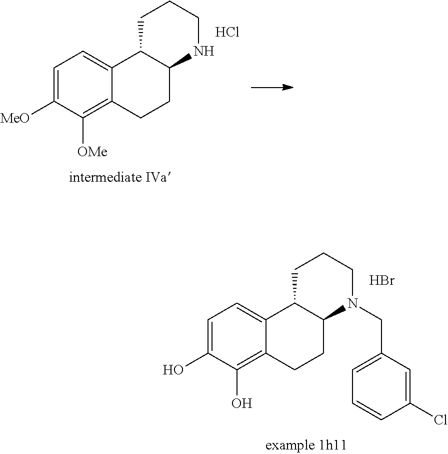

Example 1h11 (4aS,10bS)-4-(3-Chloro-benzyl)-1,2,3,4,4a,5,6,10b-octahydro-benzo[f]quinoline-7,8-diol hydrobromide or (4aR,10bR)-4-(3-chloro-benzyl)-1,2,3,4,4a,5,6,10b-octahydro-benzo[f]quinoline-7,8-diol hydrobromide. Intermediate IVa′ (0.2 g), triethyl amine (0.5 mL), and 3-chlorobenzyl chloride (0.1 mL) were stirred in 2-butanone (10 mL) at 80° C. overnight. The crude mixture was purified by filtration through a plough of silica gel (eluent: ethyl acetate/methanol 9:1) to afford an intermediate. This material was dissolved in methylene chloride (2 mL) and cooled to −78° C. BBr₃ (1M in methylene chloride; 1.5 mL) was added, and the mixture was allowed to warm to room temperature. The mixture was cooled to −60° C. before it was quenched with methanol (1.5 mL). The mixture was warmed to room temperature, and the precipitated example 1h11 (210 mg) was isolated. LC/MS (method 25): RT (UV)=1.08 min, UV-purity 99.4%, ELS-purity 99.8%, mass observed 344.5.

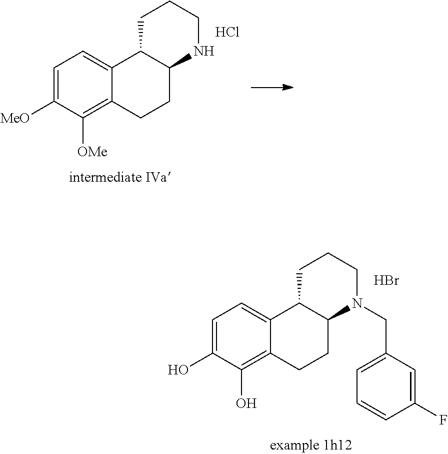

Example 1h12 (4aS,10bS)-4-(3-Fluoro-benzyl)-1,2,3,4,4a,5,6,10b-octahydro-benzo[f]quinoline-7,8-diol hydrobromide or (4aR,10bR)-4-(3-fluoro-benzyl)-1,2,3,4,4a,5,6,10b-octahydro-benzo[f]quinoline-7,8-diol hydrobromide. Intermediate IVa′ (0.2 g), triethyl amine (0.5 mL), and 3-fluorobenzyl chloride (0.1 mL) were stirred in 2-butanone (10 mL) at 80° C. overnight. The crude mixture was purified by filtration through a plough of silica gel (eluent: ethyl acetate/methanol 9:1) to afford an intermediate. This material was dissolved in methylene chloride (2 mL) and cooled to −78° C. BBr₃ (1M in methylene chloride; 1.3 mL) was added, and the mixture was allowed to warm to room temperature. The mixture was cooled to −60° C. before it was quenched with methanol (1.3 mL). The mixture was warmed to room temperature, and the precipitated example 1h12 (169 mg) was isolated. LC/MS (method 25): RT (UV)=1.00 min, UV-purity 99.8%, ELS-purity 99.8%, mass observed 328.3.

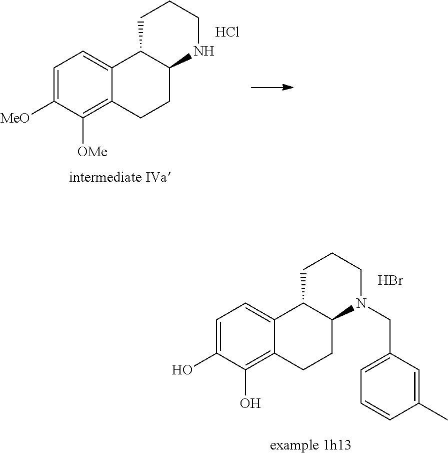

Example 1h13 (4aS,10bS)-4-(3-Methyl-benzyl)-1,2,3,4,4a,5,6,10b-octahydro-benzo[f]quinoline-7,8-diol hydrobromide or (4aR,10bR)-4-(3-methyl-benzyl)-1,2,3,4,4a,5,6,10b-octahydro-benzo[f]quinoline-7,8-diol hydrobromide. Intermediate IVa′ (0.2 g), triethyl amine (0.5 mL), and 3-methylbenzyl bromide (0.1 mL) were stirred in 2-butanone (10 mL) at 80° C. overnight. The crude mixture was purified by filtration through a plough of silica gel (eluent: ethyl acetate/methanol 9:1) to afford an intermediate. 130 mg of this material was dissolved in methylene chloride (1.5 mL) and cooled to −78° C. BBr₃ (1M in methylene chloride; 0.8 mL) was added, and the mixture was allowed to warm to room temperature. The mixture was cooled to −60° C. before it was quenched with methanol (0.6 mL). The mixture was warmed to room temperature, and the precipitated example 1h13 (58.9 mg) was isolated. LC/MS (method 25): RT (UV)=1.08 min, UV-purity 97.1%, ELS-purity 99.7%, mass observed 324.8.

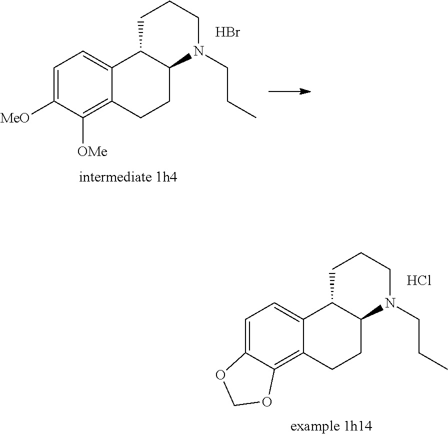

Example 1h14 (5S,10S)-4-Propyl-1,2,3,4,5,6,7,10-octahydro-15,17-dioxa-4-aza-cyclopenta[a]phenanthrene hydrochloride or (5R,10R)-4-propyl-1,2,3,4,5,6,7,10-octahydro-15,17-dioxa-4-aza-cyclopenta[a]phenanthrene hydrochloride. Example 1h4 (2×350 mg), cesium carbonate (2×815 mg), and bromo-chloro-methane (2×100 microL) were mixed with DMF (2×4 mL) in two microwave vials. The vials were heated to 100° C. under microwave conditions. The two crude mixtures were combined and purified by chromatography (eluent: chloroform→chloroform/methanol 9:1) to afford an intermediate. This material was dissolved in ethanol (2 mL) and treated with HCl in diethyl ether to precipitate example 1h14 (305 mg). LC/MS (method 336): RT (UV)=0.43 min, UV-purity 97%, ELS-purity 100%, mass observed 374.7.

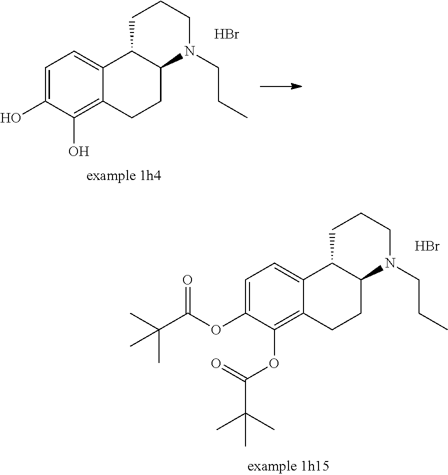

Example 1h15 2,2-Dimethyl-propionic acid (4aS,10bS)-8-(2,2-dimethyl-propionyloxy)-4-propyl-1,2,3,4,4a,5,6,10b-octahydro-benzo[f]quinolin-7-yl ester hydrobromide or 2,2-dimethyl-propionic acid (4aR,10bR)-8-(2,2-dimethyl-propionyloxy)-4-propyl-1,2,3,4,4a,5,6,10b-octahydro-benzo[f]quinolin-7-yl ester hydrobromide. Example 1h4 (0.4 g) was dissolved in trifluoroacetic acid (15 mL) and pivaloyl chloride (450 mg) was added portion-wise, and the mixture was stirred at room temperature over the weekend. The crude mixture was concentrated in vacuo. The residue was dissolved in ethanol (ca 2 mL) and treated with diethyl ether to precipitate example 1h15 (425 mg) as a white solid. LC/MS (method 336): RT (UV)=0.9 min, UV-purity 100%, ELS-purity 100%, mass observed 430.6.

### Abbreviations and List of Chemicals Used

The following abbreviations are used. This paragraph also outlines the chemicals used along with their commercial source (not included for standard solvents).

AcCl=acetyl chloride (e.g. Aldrich 23, 957-7). ACh=acetylcholine. AcOH=acetic acid. AD=Alzheimer's disease. ADME=absorption-distribution-metabolism-excretion. Allyl bromide (e.g. Fluka 05870) AlCl₃=aluminium chloride (e.g. Aldrich 29, 471-3). α_{D}=specific optical rotation. BBr₃=boron tribromide (used as DCM solution; Aldrich 17, 893-4). Boc₂O=Boc anhydride/di-t-butyl dicarbonate (e.g. Aldrich 19, 913-3). Brine=saturated aqueous solution of sodium chloride. BSA=bovine serum albumin. (s-Butyl)lithium (used as a cyclo-hexane solution; e.g. Aldrich 19, 559-6). cAMP=cyclic adenosine monophosphate. Celite=filter-aid. CH₂BrCl=bromochloromethane (Aldrich 13, 526-7). CH₃I=methyl iodide/iodomethane (e.g. Aldrich 28, 956-6). CHO cell=Chinese hamster ovary cell. ClAcCl=chloroacethyl chloride (e.g. Aldrich 10, 449-3). Cs₂CO₃=cesium carbonate (Aldrich 441902). CuI=copper(I)iodide (Aldrich 215554). Cyclobutanone (e.g. Aldrich C9, 600-1). cyclo-propyl methyl bromide/(bromomethyl)-cyclo-propane (Aldrich 24, 240-3). DA=dopamine. D1=dopamine D1 receptor. D2=dopamine D2 receptor. D3=dopamine D3 receptor. D4=dopamine D4 receptor. D5=dopamine D5 receptor. DCM=dichloro-methane/methylene chloride. 1,6-dibromo-2-naphthol (e.g. Aldrich D4, 180-5). DMF=dimethyl formamide. DMSO=dimethyl sulfoxide. L-DOPA=(levo)-3,4-dihydroxy phenylalanine DOPAC=3,4-dihydroxyphenyl acetic acid (DA metabolite). EC₅₀=concentration required to induce a response halfway between the baseline and the maximum response for the compound in question. ELSD=evaporative light scattering detection. Et₃N=triethyl amine. Et₂NH=diethyl amine. EtOAc=ethyl acetate. Ethyl 2-chloro-nicotinate (e.g. ABCR AV20359). 99% EtOH=absolute ethanol. Ethyl magnesium bromide (used as a 3 M solution in Et₂O; Aldrich 18, 987-1). Et₂O=diethyl ether. [(1-Ethoxycyclopropyl)-oxy]trimethylsilane (Aldrich 332739). Ethylene glycol=1,2-ethanediol. 35% H₂O₂=35% aqueous solution of hydrogen peroxide (e.g. Aldrich 34, 988-7). FLIPR=fluorometric imaging plate reader. FSB=foetal bovine serum. h=hours. 48% HBr=48% aqueous solution of hydrogen bromide. 18%/37% HCl=18%/37% aqueous solution of hydrogen chloride. 1 M HCl/2 M HCl=1 M/2 M aqueous solution of hydrogen chloride (unless noted specifically as a 2M Et₂O solution, which is commercially available, e.g. Aldrich 45, 518-0). HMPA=hexamethylphosphorous triamide. HVA=homovanillic acid (DA metabolite). i=iso. IBMX=3-i-butyl-1-methylxanthine. i.d.=inner diameter. 1-Iodopropane (e.g. Aldrich 17, 188-3). K₂CO₃=potassium carbonate (e.g. Aldrich 20, 961-9). KMnO₄=potassium permanganate (e.g. Aldrich 39, 912-4). KO=knock-out. LDA=lithium di-1-propylamide (used as a THF/heptane/ethylbenzene solution; Fluka 62491). LC/MS=high-performance liquid chromatography/mass spectrometer. LAH=lithium aluminium hydride (used as a 1M THF solution; Aldrich 21, 277-6). LiCl=lithium chloride (e.g. Aldrich 31,046-8). L-Selectride=lithium tri-s-butylborohydride (used as a 1M THF solution; Aldrich 17, 849-7). MDO=methylene-di-oxy. MED=minimal effective dose. MED_{Nemonapride}=minimal effective dose in the presence of Nemonapride. MeOH=methanol. methoxyacetyl chloride (e.g. Aldrich M965-3). min=minutes. MBD=minimal brain dysfunction. 2-Methyl-THF (e.g. Aldrich 41, 424-7). MPTP=1-methyl-4-phenyl-1,2,3,6-tetrahydropyridine. MTBE=methyl t-butyl ether. n=normal. NaCNBH₃=sodium cyanoborohydride (Aldrich 15, 615-9). Na₂S₂O₃=Sodium bisulfite (used as an 38-40% aqueous solution; eg. Riedel 13438). NaH=sodium hydride (used as a 60% dispersion; Aldrich 45, 291-2). NaIO₄=sodium periodate (e.g. Aldrich 31, 144-8). 1 M/9 M NaOH=1 M/9 M aqueous solution of sodium hydroxide. NaOMe=sodium methoxide (used as a ca. 5 M solution in methanol; e.g. Aldrich 15, 625-6). NPA=N-n-propyl Apomorphine. 6-OHDA=6-hydroxydopamine. PBS=phosphate buffered saline (0.02 M sodium phosphate buffer with 0.15 M sodium chloride, pH adjusted to 7.4). PD=Parkinson's disease. PFC=prefrontal cortex. Pd/C=palladium-on-charcoal (e.g. Aldrich 20, 569-9). Pd(OAc)₂=palladium(II)acetate (Alfa Aesar 010516). Piperonyl alcohol (e.g. Aldrich P4, 940-6). PK=pharmaco-kinetic. PLMD=periodic limb movement disorder. Propargyl chloride (e.g. Aldrich 14, 399-5). Propionaldehyde (e.g. Aldrich 58, 812-4). PTSA=para-toluene sulfonic acid hydrate (e.g. Aldrich 40, 288-5). PivCl=pivaloyl chloride/trimethyl acetyl chloride (e.g. Aldrich T7, 260-5). RLS=restless legs syndrome. rt=room temperature. RT=retention time. s=secondary. sat. NaHCO₃=saturated aqueous solution of sodium hydrogen carbonate. sat. NH₄C1=saturated aqueous solution of ammonium chloride. SC=subcutaneous. SFC=supercritical flash chromatography. Sodium metal (e.g. Aldrich 28, 205-7). t=tertiary. TBAI=tetra-n-butyl ammonium iodide (e.g. Aldrich 14,077-5). TFA=trifluoroacetic acid. TFAA=trifluoroacetatic acid anhydride. THF=tetrahydrofuran (dried over 4 Å molecular sieves). TLC=thin layer chromatography. CH(OCH₃)₃=trimethyl orthoformate (e.g. Aldrich 30, 547-2). UV=ultraviolet purity (at 254 nm unless noted differently).

### Pharmacological Testing

D1 cAMP Assay

The ability of the compounds to either stimulate or inhibit the D1 receptor mediated cAMP formation in CHO cells stably expressing the human recombinant D1 receptor was measured as follows. Cells were seeded in 96-well plates at a concentration of 11000 cells/well 3 days prior to the experiment. On the day of the experiment the cells were washed once in preheated G buffer (1 mM MgCl₂, 0.9 mM CaCl₂, 1 mM IBMX (3-i-butyl-1-methylxanthine) in PBS (phosphate buffered saline)) and the assay was initiated by addition of 100 micro-L of a mixture of 30 nM A68930 and test compound diluted in G buffer (antagonism) or test compound diluted in G buffer (agonism).

The cells were incubated for 20 minutes at 37° C. and the reaction was stopped by the addition of 100 micro-L S buffer (0.1 M HCl and 0.1 mM CaCl₂) and the plates were placed at 4° C. for 1 h. 68 micro-L N buffer (0.15 M NaOH and 60 mM NaOAc) was added and the plates were shaken for 10 minutes. 60 micro-1 of the reaction were transferred to cAMP FlashPlates (DuPont NEN) containing 40 micro-L 60 mM Sodium acetate pH 6.2 and 100 micro-L IC mix (50 mM Sodium acetate pH 6.2, 0.1% sodium azide, 12 mM CaCl₂, 1% BSA (bovine serum albumin) and 0.15 micro-Ci/mL ¹²⁵I-cAMP) were added. Following an 18h incubation at 4° C. the plates were washed once and counted in a Wallac TriLux counter.

D2 cAMP Assay

The ability of the compounds to either stimulate or inhibit the D2 receptor mediated inhibition of cAMP formation in CHO cells transfected with the human D2 receptor was measure as follows. Cells were seeded in 96 well plates at a concentration of 8000 cells/well 3 days prior to the experiment. On the day of the experiment the cells were washed once in preheated G buffer (1 mM MgCl₂, 0.9 mM CaCl₂, 1 mM IBMX in PBS) and the assay was initiated by addition of 100 micro-1 of a mixture of 1 micro-M quinpirole, 10 microM forskolin and test compound in G buffer (antagonism) or 10 micro-M forskolin and test compound in G buffer (agonism).

The cells were incubated 20 minutes at 37° C. and the reaction was stopped by the addition of 100 micro-1 S buffer (0.1 M HCl and 0.1 mM CaCl₂) and the plates were placed at 4° C. for 1 h. 68 micro-L N buffer (0.15 M NaOH and 60 mM Sodium acetate) were added and the plates were shaken for 10 minutes. 60 micro-L of the reaction were transferred to cAMP FlashPlates (DuPont NEN) containing 40 micro-L 60 mM NaOAc pH 6.2 and 100 micro-L IC mix (50 mM NaOAc pH 6.2, 0.1% Sodium azide, 12 mM CaCl₂, 1% BSA and 0.15 micro-Ci/ml ¹²⁵I-cAMP) were added. Following an 18h incubation at 4° C. the plates were washed once and counted in a Wallac TriLux counter.

### D5 Assay

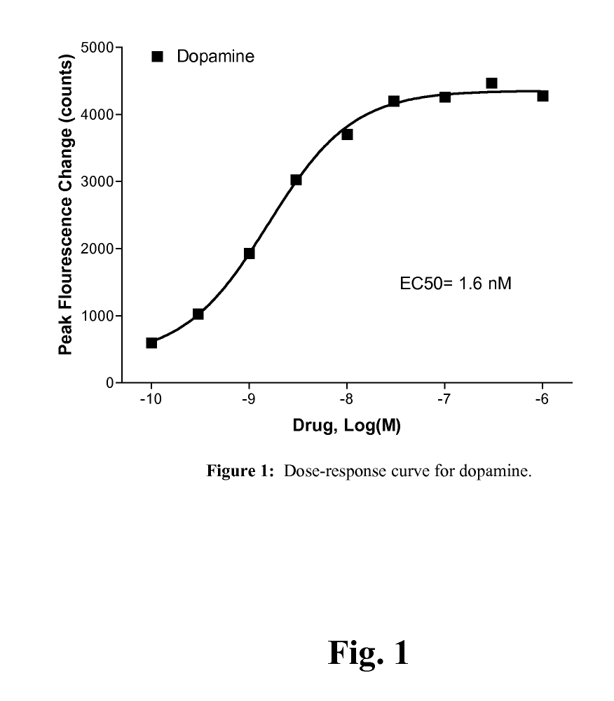

Concentration-dependent stimulation of intracellular Ca²⁺ release by dopamine in hD5-transfected CHO-Ga16 cells. The cells were loaded with fluoro-4, a calcium indicator dye, for 1 h. Calcium response (fluorescence change) was monitored by FLIPR (fluorometric imaging plate reader) for 2.5 min. Peak responses (EC₅₀) were averaged from duplicate wells for each data point and plotted with drug concentrations (cf. FIG. 1 for dopamine).

Concentration effects curves to agonists were constructed by adding different concentrations to different wells using a Fluorescence Imaging Plate Reader (FLIPR™) (Molecular Devices, Sunnyvale, Calif.). Curves were fitted with sigmoidal dose response equation I=Iₘₐₓ/(1+(EC₅₀/[Agonist])ⁿ), where the EC₅₀ value is the concentration of agonist that produced half-maximal activation, and n is the Hill coefficient. Fits were made using the Graphpad Prism 4 software (San Diego, Calif.).

### D1/D2 Dissections

Dopamine agonists can have activity at either the D1-like receptors, the D2-like receptors, or both. We have used the rotation response in rats with unilateral 6-OHDA lesions to assess compounds for their ability to stimulate both receptor types and induce rotation [Ungerstedt, Arbuthnott; Brain Res., 24, 485 (1970); Setler, Sarau, Zirkle, Saunders; Eur. J. Pharmacol., 50(4), 419 (1978); Ungerstedt, Herrera-Marschitz, Jungnelius, Ståhle, Tossman, Zetterström; in “Advances in Dopamine Research” (Kohsaka, Ed.), Pergamon Press, Oxford, p. 219 (1982)]. Experiments consist of determining a minimum effective dose (MED) to induce rotation for the compound in question. Once a MED has been determined, a second experiment is performed to determine the MED of the compound to overcome Nemonapride block (MED_{Nemonapride}). Nemonapride is a D2-like antagonist that blocks the D2-like receptor, therefore any observed rotations would be dependent upon activity at the D1-like receptor. Finally, once the MED_{Nemonapride} is known a third experiment is run using the MED_{Nemonapride} dose and observing the effect of the D1-like antagonist, SCH 23390 alone, the D2-like antagonist, Nemonapride alone and finally, the effect of combined treatment with SCH 23390 and Nemonapride. This third experiment confirms the activity of the compound at both receptors as either antagonist alone can only partially inhibit the rotation response induced by the test compound while the combination treatment completely blocks all rotations in the rats [Arnt, Hytell; Psychopharmacology, 85(3), 346 (1985); Sonsalla, Manzino, Heikkila; J. Pharmacol Exp. Ther., 247(1), 180 (1988)]. This model was validated using Apomorphine as the proof-of-principle compound for mixed D1-like/D2-like agonists.

The D1/D2 profile in 6-OHDA rats of some of the compounds of the invention resembles that of Apomorphine. Consequently, some of the compounds of the invention are superior to D2-agonists.

### Methods—Cell Culture

Human D5 (hD5) expression construct was made using a modified pEXJ vector. A stable cell line expressing a promiscuous human Galpha16 G protein (CHO-Ga16) was purchased from (Molecular Devices, Sunnyvale, Calif.). The cells were cultured in HAMS F-12 media (Invitrogen, Carlsbad, Calif.) containing 10% FSB (foelal bovine serum), 1% L-glutamine and 1% penicillin/streptomycin (P/S) at 37° C. in 5% CO₂. 48h before assay, CHO-Ga16 cells were transiently transfected with hD5 receptor DNA using a lipofectamine Plus method (Invitrogen, Carlsbad, Calif.), and allow to grow for 1 day in serum and P/S free media. 24 h before assay, hD5 transfected CHO-Ga16 cells were seeded at a density of 10,000 cells per well into black walled clear-base 384-well plates pretreated with poly-D-Lysine (Becton Dickinson, USA). The cells were then cultured in HAMS F-12 cell growth media containing 1.5% FBS, 1% L-glutamine and 1% penicillin/streptomycin (P/S) at 37° C. in 5% CO₂

### Methods—Intracellular Calcium Mobilization Assays

For measurements of intracellular free calcium concentration ([Ca²⁺]ᵢ), the culture medium was replaced with a freshly prepared loading buffer. The loading buffer contains 1×HBSS (Invitrogen), 20 mM HEPES (Sigma), 0.1% BSA (Sigma), 1.5 micro-M Fluoro-4-AM (Molecular Probes), and 2.5 mM probenecid (prepared fresh) (Sigma). The plates were incubated for 1 h at 37° C. and 5% CO₂ and washed three times with washing buffer. The washing buffer contains the same components as the loading buffer excluding Fluo-4-AM. The cells were then placed into a fluorescence imager plate reader (FLIPR™, Molecular Devices) to monitor cell fluorescence before and after addition of various compounds.

The compounds of interest were diluted in washing buffer to a 4× final concentration and aliquoted into a clear round-bottom plate. The dye was excited at the 488 nm wavelength using an argon ion laser and the signal was detected using the standard 510-570 nm emission [Sullivan, Tucker, Dale; Methods Mol. Biol., 114, 125 (1999)]. Concentration effects curves for agonists were constructed by adding different concentrations to different wells. Relative fluorescence is measured by subtracting basal from peak fluorescence after addition of drug. The data were then collected and analyzed using the FLIPR™ software and GraphPad Prism 4.

Antagonist activities of compounds were assayed for their inhibition of the signal elicited by agonist ligands. Cells were pre-incubated with compounds at increasing concentrations, and then stimulated with agonists using the methods described above.

In vitro Hepatocyte Assay

Cryopreserved pooled male rat hepatocytes (Sprague Dawley) and pooled human hepatocytes from 10 donors (male and female) were purchased from 1n Vitro Technologies Inc., BA, USA. Cells were thawed at 37° C. in a water bath, live cells counted and seeded in a total of 100 micro-L in Dulbecco's modified Eagle medium (high glucose) with 5 mM Hepes buffer in 96 well plates, each well containing 250.000 and 500.000 cells/mL for rat and human hepatocytes, respectively. Incubations were started after 15 min of pre-incubation and stopped at time points of 0, 5, 15, 30 and 60 min for rats and at 0, 30, 60, 90 and 120 min for human hepatocytes. Incubations were stopped by addition of an equal volumes of ice-cold acetonitrile containing 10% 1 M HCl. Following centrifugation, 20 micro-L of the supernatants were injected on a HPLC Column Atlantis dC18 3 micro-m, 150×2.1 mm i.d. (Waters, Mass., USA). The mobile phase had the following composition: A: 5% acetonitrile, 95% H₂O, 3.7 ml/l 25% aq. NH₃, 1.8 mL/L formic acid. Mobile phase B: 100% acetonitrile and 0.1% formic acid. The flow rate was 0.3 ml/min. The gradient operated from 0% to 75% B from 5 min to 20 min and the eluate was analyzed using a Q-TOFmicro mass spectrometer (Waters, Mass., USA). Formation of the product/metabolite was confirmed by accurate mass measurements and comparison with a synthesized standard giving coinciding retention times.

1. A compound having the formula I: 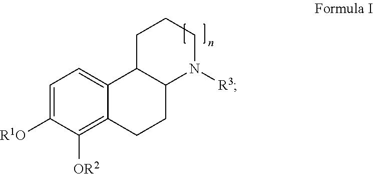
wherein is n is 0 or 1;
wherein R¹ and R² are independently selected from the group consisting of hydrogen, C₁₋₆ alkanoyl, phenylacetyl or benzoyl, or wherein R¹ and R² fuse to form a methylene (CH₂) group, a carbonyl (C═O) group or an oxalyl (O═C—C═O) group; and
wherein R³ is selected from the group consisting of hydrogen, methyl, ethyl, n-propyl, cyclopropyl, cyolobutyl, cycloalkylalkyl, allyl, propargyl, hydroxyethyl, benzyl or phenylethyl, where the benzyl and phenylethyl are optionally substituted with C₁-C₆ alkyl or halogen; or a pharmaceutically acceptable acid addition salt thereof;
with the proviso that the compound is not the racemic mixture of one of the following compounds:
1,2,3,4,4a,5,6,10b-octahydro-benzo[f]quinoline-7,8-diol,
4-methyl-1,2,3,4,4a,5,6,10b-octahydro-benzo[f]quinoline-7,8-diol,
4-ethyl-1,2,3,4,4a,5,6,10b-octahydro-benzo[f]quinoline-7,8-diol,
4-n-propyl-1,2,3,4,4a,5,6,10b-octahydro-benzo[f]quinoline-7,8-diol,
4-benzyl-1,2,3,4,4a,5,6,10b-octahydro-benzo[f]quinoline-7,8-diol, and
4-phenylethyl-1,2,3,4,4a,5,6,10b-octahydro-benzo[f]quinoline-7,8-diol.

2. The compound of claim 1, wherein R³ is selected from the group consisting of hydrogen, methyl, ethyl, n-propyl, allyl, and propargyl.

3. The compound of claim 1, wherein R³ is selected from the group consisting of cyclopropyl, cyclobutyl, and hydroxyethyl.

4. The compound of claim 1 wherein n is 0.

5. The compound of claim 1 wherein n is 1.

6. The compound of claim 1, wherein the compound is further characterized as the substantially pure trans-diastereoisomer.

7. The compound of claim 1, wherein R¹ and R² are fused and form a methylene (CH₂) group.

8. The compound of claim 1, wherein n is 0 and wherein the compound is further characterized as the substantially pure (3aS,9bR)-enantiomer.

9. The compound of claim 1, wherein n is 0 and wherein the compound is further characterized as the substantially pure (3aS,9bS)-enantiomer.

10. The compound of claim 1, wherein n is 1 and wherein the compound is further characterized as the substantially pure (4aS,10bR)-enantiomer.

11. The compound of claim 1, wherein n is 1 and wherein the compound is further characterized as the substantially pure (4aS,10bS)-enantiomer.

12. The compound of claim 1, wherein the compound is selected from the group consisting of
(6aR,10aR)-6,6a,7,8,9,10,10a,11-octahydro-1,3-dioxa-7-aza-cyclopenta[a]anthracene;
(6aR,10aR)-7-methyl-6,6a,7,8,9,10,10a,11-octahydro-1,3-dioxa-7-azacyclopenta[a]anthracene;
(6aR,10aR)-7-ethyl-6,6a,7,8,9,10,10a,11-octahydro-1,3-dioxa-7-aza-cyclopenta[a]anthracene; and
(6aR,10aR)-7-n-propyl-6,6a,7,8,9,10,10a,11-octahydro-1,3-dioxa-7-aza-cyclopenta[a]anthracene,
or a pharmaceutically acceptable acid addition salt thereof.

13. The compound of claim 1, wherein n is 0; wherein R¹ and R² are fused and form a methylene (CH₂) group; and wherein R³ is selected from the group consisting of hydrogen, methyl, ethyl and n-propyl.

14. The compound of claim 1, wherein n is 1; wherein R¹ and R² are fused and form a methylene (CH₂) group; and wherein R₃ is selected from the group consisting of hydrogen, methyl, ethyl and n-propyl.

15. (canceled)

16. A pharmaceutical composition comprising the compound of claim 1, or a pharmaceutically acceptable salt thereof, and a pharmaceutically acceptable carrier.

17-28. (canceled)
